(19) United States
(12) Patent Application Publication
(10) Pub. No.: US 2006/0134120 A1
(43) Pub. Date: Jun. 22, 2006

(54) Multiple marker assay for detection of ovarian cancer

(75) Inventor: Eleftherios P Diamandis, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto, (CA)

(21) Appl. No.: 10/544,944

(22) PCT Filed: Feb. 26, 2004
(86) PCT No.: PCT/CA04/00281

§ 371 Date: Jan. 26, 2006

Related U.S. Application Data
(60) Provisional application No. 60/450,406, filed on Feb. 26, 2003.

(51) Int. Cl. A61K 39/395; G01N 33/574

(52) U.S. Cl. 424/155.1; 435/7.23

(57) ABSTRACT

Methods for diagnosing and monitoring ovarian cancer in a subject comprising measuring a plurality of kallikrein polypeptides, and optionally CA125, or nucleic acids encoding the polypeptides in a sample from the subject. The kallikrein polypeptides include kallikrein 5, kallikrein 6, kallikrein 7, kallikrein 8, kallikrein 10 and kallikrein 11.

### FIELD OF THE INVENTION

The invention relates to compositions, kits, and methods for detecting, characterizing, preventing, and treating ovarian cancer.

### BACKGROUND OF THE INVENTION

Epithelial ovarian carcinoma is the most common and most lethal of all gynecologic malignancies. Only 30% of ovarian tumors are diagnosed at an early stage (Stage I/II), when survival rates reach 90%. The rest are diagnosed at an advanced stage, with survival rates of less than 20% (Greenlee R T, Hill-Harmon M B, Murray T, et al., 2001. CA Cancer J Clin.2001;51:15-36). Currently, the only well-accepted serological marker is CA 125, a large glycoprotein of unknown function (Meyer T, Rustin G J., Br J Cancer 2000;82:1535-1538). However, CA125 has limitations as a diagnostic, prognostic and screening tool (Holschneider C H, Berek J S, Semin Surg Oncol. 2000;19:3-10). Consequently, there is a need to enhance the overall diagnostic/prognostic capability of CA 125.

Kallikreins are a subgroup of secreted serine proteases, encoded by highly conserved and tightly clustered multigene families in humans, rats and mice. The human kallikrein gene family resides on chromosome 19q13.4 and is comprised of 15 members, whose genes are designated as KLK1 to KLK15 and the corresponding proteins as hK1 to hK15 (Yousef G M, Diamandis E P., Endocr Rev. 2001;22:184-2041; Yousef G M, Chang A, Scorilas A, et al., Biochem Biophys Res Commun. 2000;276:125-133; Diamandis E P, Yousef G M, Clements J, et al. Clin Chem. 2000;46:1855-1858). Kallikreins are expressed in a wide variety of tissues and are found in many biological fluids (e.g. cerebrospinal fluid, serum, seminal plasma, milk, etc.) where they are predicted to process specific substrates. Kallikreins may participate in cascade reactions similar to those involved in digestion, fibrinolysis, coagulation, wound healing and apoptosis ((Yousef G M, Diamandis E P., Endocr Rev. 2001;22:184-2041). Many kallikreins have been found to be differentially expressed in endocrine-related malignancies (Diamandis E P, Yousef G M, Expert Rev. Mol. Diagn. 2001;1:182-190), including prostate (Barry M J. Clinical practice, N Engl J Med. 2001;344:1373-1377; Rittenhouse H G, Finlay J A, Mikolajczyk S D, et al., Crit Rev Clin Lab Sci. 1998;35:275-368; and Yousef G M, Scorilas A, Jung K, et al., J Biol Chem. 2001;276:53-61), ovarian (Kim H, Scorilas A, Katsaros D, et al., Br J Cancer, 2001;84:643-650; Anisowicz A, Sotiropoulou G, Stenman, et al., Mol Med. 1996;2:624-636; Tanimoto H, Underwood L J, Shigemasa K, et al., Cancer. 1999;86:2074-2082; Magklara A, Scorilas A, Katsaros D, et al., Clin Cancer Res. 2001;7:806-811; Yousef G M, Kyriakopoulou L G, Scorilas A, et al., Cancer Res. 2001;61:7811-7818; Luo L, Bunting P, Scorilas A, Diamandis E P., Clin Chim Acta. 2001;306:111-118), breast (Yousef G M, Magklara A, Chang A, et al., Cancer Res. 2001;61:3425-3431;Yousef G M, Chang A, Diamandis B P; J Biol Chem. 2000;275:11891-11898; and Yousef G M, Magklara A, Diamandis E P, Genomics. 2000;69:331-341), and testicular cancer (Luo L Y, Rajpert-De Meyts E R, Jung K, et al., 2001;85:220-²²⁴). In addition, many kallikrein genes examined thus far are under steroid hormone regulation, implicating a role for kallikreins in endrocrine-related tissues (Yousef G M, Diamandis E P., Endocr Rev., 2001;22:184-204). Furthermore, hK6, hK10 and hK11 have been recently identified as novel serological ovarian cancer biomarkers (Luo L, Bunting P, Scorilas A, Diamandis E P., Clin Chim Acta. 2001;306:111-118 Diamandis E P, Yousef G M, Soosaipillai A R Bunting P., Clin Biochem. 2000;33:579-583, and Diamandis E P, Okui A, Mitsui S, et al., Cancer Res. 2002;62:295-300).

### SUMMARY OF THE INVENTION

The present invention seeks to overcome the drawbacks inherent in the prior art and seeks to provide sensitive and accurate multimarker methods for the detection of ovarian cancer. A plurality of kallikrein polypeptides and polynucleotides encoding the polypeptides, optionally in combination with CA125 and polynucleotides encoding CA125 can have particular application in the detection of ovarian cancer. A plurality of kallikrein markers (i.e. two or more of kallikrein 5, kallikrein 6, kallikrein 7, kallikrein 8, kallikrein 10, and kallikrein 11) and polynucleotides encoding the polypeptides, optionally in combination with CA125 and polynucleotides encoding CA125, constitute biomarkers for the diagnosis, monitoring, progression, treatment, and prognosis of ovarian cancer, and they may be used as biomarkers before surgery or after relapse.

In accordance with the methods of the invention, the presence of levels of markers in a sample can be assessed, for example by detecting the presence in the sample of (a) polypeptides or polypeptide fragments corresponding to the markers; (b) metabolites which are produced directly or indirectly by polypeptides corresponding to the markers; (c) transcribed nucleic acids or fragments thereof having at least a portion with which the markers are substantially identical; and/or (c) transcribed nucleic acids or fragments thereof, wherein the nucleic acids hybridize with the markers.

In an aspect of the invention, a method is provided for detecting ovarian cancer in a patient comprising detecting a plurality of kallikrein polypeptides, optionally in combination with CA125, in a sample from the patient wherein the method provides substantially increased sensitivity compared to methods using CA125 alone. In an embodiment, sensitivity is increased by at least 0.5%, 1%, 2%, 3%, 4%, 5%,10%,15%,20%,25%,30%, and 35% compared to using CA125 alone.

In an embodiment, the invention provides a method for detecting a plurality of kallikrein markers, and optionally CA125, associated with ovarian cancer in a patient comprising:
(a) obtaining a sample from a patient;
(b) detecting or identifying in the sample kallilcrein markers, optionally in combination with CA125, wherein the kallikrein markers comprise or are selected from the group consisting of kallikrein 5, kallikrein 6, kallikrein 7, kallikrein 8, kallikrein 10, and kallikrein 11; and
(c) comparing the detected amounts with amounts detected for a standard.

The term “detect” or “detecting” includes assaying, assessing, imaging or otherwise establishing the presence or absence of the target kallikrein and CA125 polypeptides or polynucleotides encoding the polypeptides, subunits thereof, or combinations of reagent bound targets, and the like, or assaying for, imaging, ascertaining, establishing, or otherwise determining one or more factual characteristics of ovarian cancer, metastasis, stage, or similar conditions. The term encompasses diagnostic, prognostic, and monitoring applications. The kallikrein polypeptides and CA125 can be detected individually, sequentially, or simultaneously.

According to a method involving kallikrein markers optionally in combination with CA125, the levels in the sample of the kallikrein markers (2, 3, 4, 5, or 6) and optionally CA125, wherein the markers comprise or are selected from kallikrein 5, kallikrein 6, kallikrein 7, kallikrein 8, kallikrein 10, and kallikrein 11, are compared with the normal levels of the kallikrein markers, and optionally CA125, in samples of the same type obtained from controls (e.g. samples from individuals not afflicted with ovarian cancer). Significantly different levels in the sample of the kallkrein markers (and optionally CA125) relative to the normal levels in a control is indicative of ovarian cancer.

In an embodiment, the invention provides a method for diagnosing and monitoring ovarian carcinoma in a subject comprising detecting in a sample from the subject kallikrein markers, and optionally CA125, wherein the kallikrein markers comprise or arc selected from the group consisting of kallikrein 5, kallikrein 6, kallikrein 7, kallikrein 8, kallikrein 10, and kallikrein 11. The kallikrein markers and CA125 can be detected using antibodies that bind to the kallikrein markers and CA125 or parts thereof.

Thus, the invention provides a method of assessing whether a patient is afflicted with or has a pre-disposition for ovarian cancer, the method comprising comparing:
(a) levels of kallikrein markers, and optionally CA125, in a sample from the patient, wherein the kallikrein markers comprise kallikrein 5, kallikrein 6, kallikrein 7, kallikrein 8, kallikrein 10, and kallikrein 11; and
(b) normal levels of kallikrein markers, and optionally CA125, in samples of the same type obtained from control patients not afflicted with ovarian cancer, wherein significantly different levels of the kallikrein markers and optionally CA125, relative to the corresponding normal levels of the kallikrein markers, and optionally CA125, is an indication that the patient is afflicted with ovarian cancer.

In an embodiment of a method of assessing whether a patient is afflicted with ovarian cancer (e.g. screening, detection of a recurrence, reflex testing), the method comprises comparing:
(a) levels of kallikrein markers, and optionally CA125, in a patient sample, wherein the kallikrein markers comprise or are selected from the group consisting of kallikrein 5, kallikrein 6, kallikrein 7, kallikrein 8, kallikrein 10, and kallikrein 1 1; and
(b) normal levels of the kallikrein markers, and optionally CA125, in a control non-ovarian cancer sample.

A significant difference between the levels of the kallikrein markers, and optionally CA125, in the patient sample and the normal levels is an indication that the patient is afflicted with ovarian cancer.

The invention further relates to a method of assessing the efficacy of a therapy for inhibiting ovarian cancer in a patient. This method comprises comparing:
(a) levels of kallikrein markers, and optionally CA125, in a first sample obtained from the patient prior to providing at least a portion of the therapy to the patient, wherein the kallikrein markers comprise or are selected from the group consisting of kallikrein 5, kallikrein 6, kallikrein 7, kallikrein 8, kallikrein 10, and kallikrein 11; and
(b) levels of the kallikrein markers, and optionally CA125, in a second sample obtained from the patient following therapy.

A significant difference between the levels of the kallikrein markers, and optionally CA125, in the second sample, relative to the first sample, is an indication that the therapy is efficacious for inhibiting ovarian cancer.

The “therapy” may be any therapy for treating ovarian cancer including but not limited to chemotherapy, immunotherapy, gene therapy, radiation therapy, and surgical removal of tissue. Therefore, the method can be used to evaluate a patient before, during, and after therapy, for example, to evaluate the reduction in tumor burden.

In an aspect, the invention provides a method for monitoring the progression of ovarian cancer in a patient, the method comprising:
(a) detecting in a patient sample at a first time point, kallikrein markers, and optionally CA125, wherein the kallikrein markers comprise or are selected from the group consisting of kallikrein 5, kallikrein 6, kallikrein 7, kallikrein 8, kallikrein 10, and kallikrein 11; and
(b) repeating step (a) at a subsequent point in time; and
(c) comparing the levels detected in (a) and (b), and therefrom monitoring the progression of ovarian cancer in the patient.

In another aspect, the invention provides a method for assessing the aggressiveness or indolence of ovarian cancer (e.g. staging), the method comprising comparing:
(a) levels of kallikrein markers, and optionally CA125, in a patient sample, wherein the kallikrein markers comprise or are selected from the group consisting of kallikrein 5, kallikrein 6, kallikrein 7, kallikrein 8, kallikrein 10, and kallikrein 11; and
(b) normal levels of the kallikrein markers, and optionally CA125 in a control sample.

A significant difference between the levels in the sample and the normal levels is an indication that the cancer is aggressive or indolent.

The invention provides a method for determining whether ovarian cancer has metastasized or is likely to metastasize in the future, the method comprising comparing:
(a) levels of kallikrein markers, and optionally CA125, in a patient sample, wherein the kallikrein markers comprise or are selected from the group consisting of kallikrein 5, kallikrein 6, kallikrein 7, kallikrein 8, kallikrein 10, and kallikrein 11; and
(b) normal levels (or non-metastatic levels) of the kallikrein markers, and optionally CA125, in a control sample.

A significant difference between the levels in the patient sample and the normal levels is an indication that the cancer has metastasized or is likely to metastasize in the future.

The invention also provides a method for assessing the potential efficacy of a test agent for inhibiting ovarian cancer in a patient, and a method of selecting an agent for inhibiting ovarian cancer in a patient.

The invention further provides a method of inhibiting ovarian cancer in a patient comprising:
(a) obtaining a sample comprising cancer cells from the patient;
(b) separately maintaining aliquots of the sample in the presence of a plurality of test agents;
(c) comparing levels of kallikrein markers, and optionally CA125, in each of the aliquots, wherein the kallikrein markers comprise or are selected from the group consisting of kallikrein 5, kallikrein 6, kallikrein 7, kallikrein 8, kallikrein 10, and kallikrein 11;
(d) administering to the patient at least one of the test agents which alters the levels of the kallikrein markers, and optionally CA125, in the aliquot containing that test agent, relative to other test agents.

The invention also contemplates a method of assessing the ovarian carcinogenic potential of a test compound comprising:
(a) maintaining separate aliquots of ovarian cells in the presence and absence of the test compound; and
(b) comparing levels of kallikrein markers, and optionally CA125, in each of the aliquots, wherein the markers comprise or are selected from the group consisting of kallikrein 5, kallikrein 6, kallikrein 7, kallikrein 8, kallikrein 10, and kallikrein 11.

A significant difference between the levels of the kallikrein markers, and optionally CA125, in the aliquot maintained in the presence of (or exposed to) the test compound relative to the aliquot maintained in the absence of the test compound, indicates that the test compound possesses ovarian carcinogenic potential.

In preferred embodiments of the methods of the invention, the kallikrein markers comprise a plurality of kallikrein markers, for example, at least three, four, five, or six of the markers. In particular, a plurality of kallikrein markers may be selected from the group consisting of kallikrein 5, kallikrein 7, kallikrein 8, and kallikrein 10, from the group consisting of kallikrein 7, kallikrein 8, kallikrein 10, and kallikrein 11, or from the group consisting of kallikrein 5, kallikrein 6, kallikrein 7, kallikrein 8, kallikrein 10 and kallikrein 11.

Other methods of the invention employ one or more polynucleotides capable of hybridizing to polynucleotides encoding kallikrein markers, and optionally CA125. Methods for detecting polynucleotides encoding a kallikrein markers, and optionally CA125, can be used to monitor ovarian cancer by detecting the nucleic acids.

Thus, the present invention relates to a method for diagnosing and monitoring ovarian cancer in a sample from a subject comprising isolating nucleic acids, preferably mRNA, from the sample; and detecting polynucleotides encoding kallikrein markers, and optionally CA125, in the sample. The presence of different levels of polynucleotides encoding kallikrein markers, and optionally CA125, in the sample compared to a standard or control is indicative of disease, disease stage, and/or prognosis, e.g. longer progression-free and overall survival.

In an embodiment, the invention provides methods for determining the presence or absence of ovarian cancer in a subject comprising (a) contacting a sample obtained from the subject with oligonucleotides that hybridize to polynucleotides encoding kallikrein markers, and optionally CA125; and (b) detecting in the sample levels of nucleic acids that hybridize to the polynucleotides relative to a predetermined cut-off value, and therefrom determining the presence or absence of ovarian cancer in the subject. Within certain embodiments, mRNA is detected via polymerase chain reaction using, for example oligonucleotide primers that hybridize to polynucleotides encoding kallikrein markers, and optionally CA125, or complements of such polynucleotides. Within other embodiments, the amount of mRNA is detected using a hybridization technique, employing oligonucleotide probes that hybridize to polynucleotides encoding kallikrein markers, and optionally CA125, or complements of such polynucleotides.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of polynucleotides encoding kallikrein markers, and optionally CA125, in the sample. For mRNA the analyzing step may be accomplished using Northern Blot analysis to detect the presence of polynucleotides encoding kallikrein markers, and optionally CA125. The analysis step may be further accomplished by quantitatively detecting the presence of polynucleotides encoding kallikrein markers, and optionally CA125, in the amplification product, and comparing the quantity of markers detected against a panel of expected values for the known presence or absence of the kallikrein markers in normal and malignant tissue derived using similar primers.

In embodiments of the methods of the invention, a plurality (eg. three, four, five or six) polynucleotides encoding kallikrein polypeptides are employed. In particular, a plurality of polynucleotides encoding kallikrein markers may be selected from the group consisting of polynucleotides encoding (i) kallikrein 5, kallikrein 7, kallikrein 8, and kallikrein 10; (ii) polynucleotides encoding kallikrein 7, kallikrein 8, kallikrein 10, and kallikrein 11; and (iii) polynucleotides encoding kallikrein 5, kallikrein 6, kallikrein 7, kallikrein 8, kallikrein 10 and kallikrein 11.

The invention also provides a diagnostic composition comprising a plurality of kallikrein polypeptides and optionally CA125 polypeptide, or polynucleotides encoding the polypeptides, or agents that bind to the polypeptides or polynucleotides.

In an embodiment, the composition comprises probes that specifically hybridize to polynucleotides encoding kallikrein markers, and optionally CA125, or fragments thereof. In another embodiment a composition is provided comprising specific primer pairs capable of amplifying polynucleotides encoding kallikrein markers, and optionally CA125, using polymerase chain reaction methodologies. In a still further embodiment, the composition comprises agents that bind to kallikrein markers, and optionally CA125, (e.g. antibodies) or fragments thereof. Probes, primers, and agents can be labeled with detectable substances.

In an aspect the invention provides an in vivo method comprising administering to a subject agents that have been constructed to target kallikrein markers, and optionally CA125.

The invention therefore contemplates an in, vivo method comprising administering to a mammal imaging agents that carry labels for imaging and that bind to kallikrein markers, and optionally CA125, and then imaging the mammal.

Still further the invention relates to therapeutic applications for ovarian cancer employing kallikrein markers, and optionally CA125, nucleic acids encoding the polypeptides, and/or agents identified using methods of the invention.

The invention also includes kits for carrying out methods of the invention. In an embodiment, the kit is for assessing whether a patient is afflicted with ovarian cancer and it comprises reagents for assessing kallikrein markers, and optionally CA125, wherein the kallikrein markers comprise or are selected from the group consisting of kallikrein 5, kallikrein 6, kallikrein 7, kallikrein 8, kallikrein 10, and kallikrein 11.

In another aspect the invention relates to a kit for assessing the suitability of each of a plurality of test compounds for inhibiting ovarian cancer in a patient. The kit comprises reagents for assessing kallikrein markers, and optionally CA125, wherein the markers comprise or are selected from the group consisting of kallikrein 5, kallikrein 6, kallikrein 7, kallikrein 8, kallikrein 10, and kallikrein 11. The kit may also comprise a plurality of test agents or compounds.

The invention contemplates a kit for assessing the presence of ovarian cancer cells, wherein the kit comprises antibodies specific for selected kallikrein markers, and optionally CA125, wherein the markers comprise or are selected from the group consisting of kallikrein 5, kallikrein 6, kallikrein 7, kallikrein 8, kallikrein 10, and kallikrein 11.

Additionally the invention provides a kit for assessing the ovarian carcinogenic potential of a test compound. The kit comprises ovarian cells and reagents for assessing kallikrein markers, and optionally CA125, wherein the markers comprise or are selected from the group consisting of kallikrein 5, kallikrein 6, kallikrein 7, kallikrein 8, kallikrein 10, and kallikrein 11.

In an aspect the invention provides a method of treating a patient afflicted with ovarian cancer comprising providing to cells of a patient antisense oligonucleotides complementary to polynucleotides encoding kallikrein markers, and optionally CA125, which are overexpressed in ovarian cancer. In an alternative method, expression of genes corresponding to kallikrein markers, and optionally CA125, which are underexpressed in ovarian cancer are increased.

The invention relates to a method of inhibiting ovarian cancer in a patient at risk for developing ovarian cancer comprising inhibiting or increasing expression (or overexpression) of genes encoding kallikrein markers and optionally CA125, wherein the markers comprise or are selected from the group consisting of kallikrein 5, kallikrein 6, kallikrein 7, kallikrein 8, kallikrein 10, and kallikrein 11, that are either overexpressed or underexpressed, in ovarian cancer.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which

FIG. 1 is a graph showing hk5 concentration in serum from non-cancer and cancer patients.

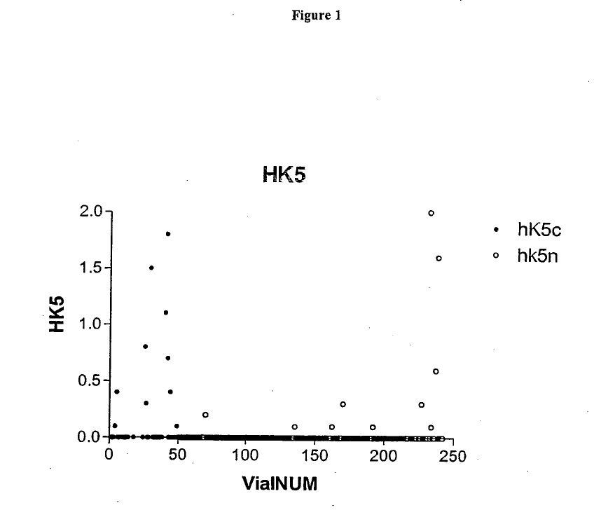

FIG. 2 is a graph showing hk6 concentration in serum from non-cancer and cancer patients.

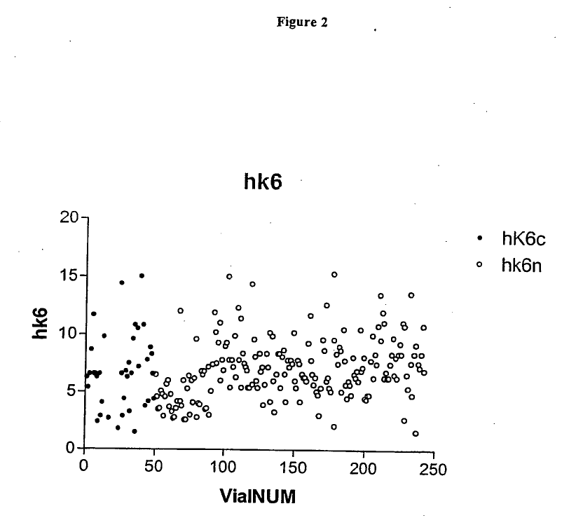

FIG. 3 is a graph showing hk7 concentration in serum from non-cancer and cancer patients.

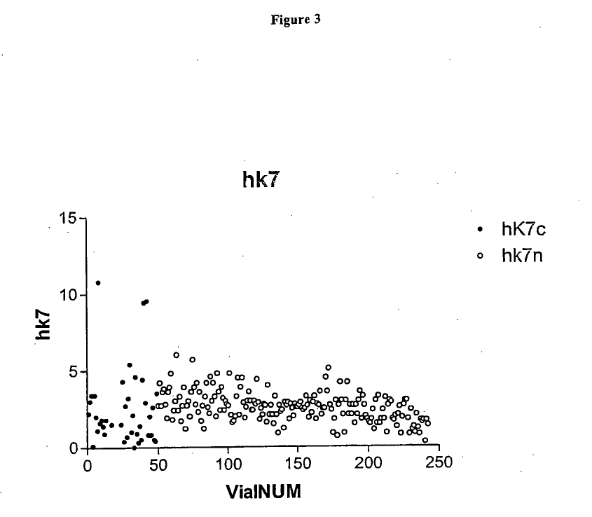

FIG. 4 is a graph showing hk8 concentration in serum from non-cancer and cancer patients.

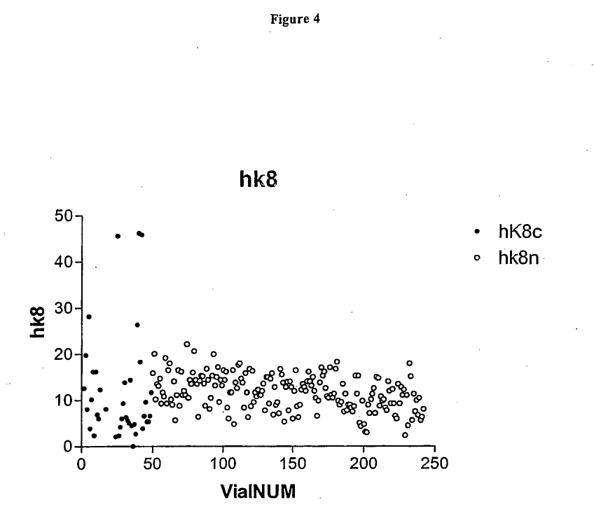

FIG. 5 is a graph showing hk10 concentration in serum from non-cancer and cancer patients.

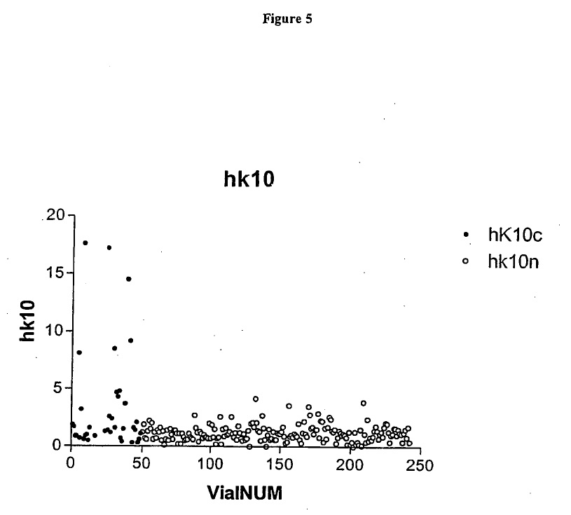

FIG. 6 is a graph showing hk11 concentration in serum from non-cancer and cancer patients.

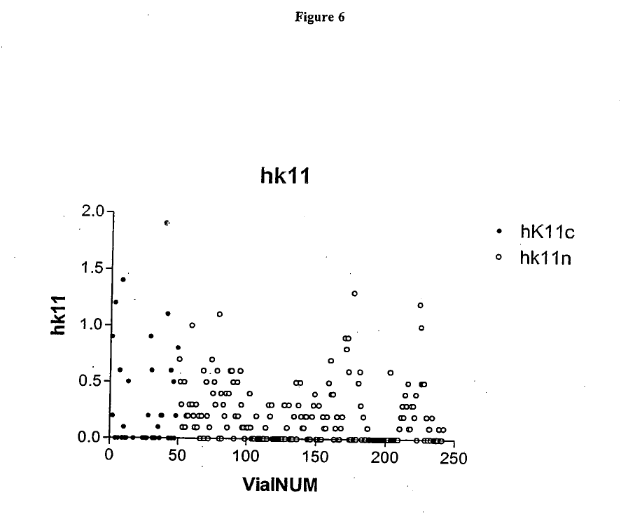

FIG. 7 is a graph showing CA125 concentration in serum from non-cancer and cancer patients.

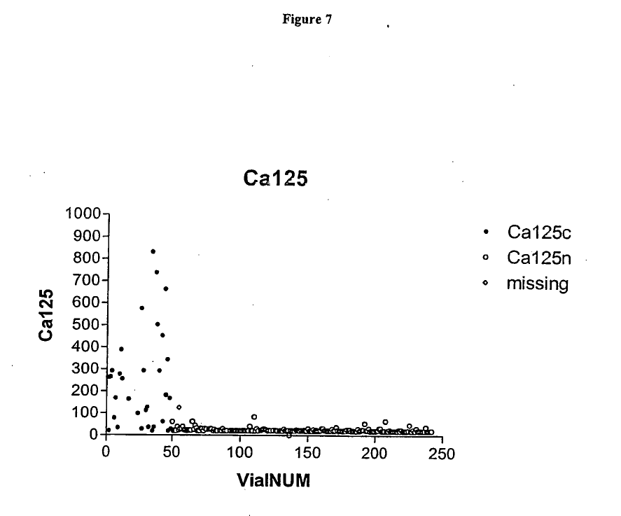

FIG. 8 is a ROC curve illustrating the added value of using kallikreins and CA125 together in a multivariate function.

### DETAILED DESCRIPTION OF THE INVENTION

The invention relates to newly discovered correlations between expression of certain markers and ovarian cancer. The combinations of markers described herein may provide sensitive methods for detecting ovarian cancer. The levels of expression of a combination of markers described herein may correlate with the presence of ovarian cancer or a pre-malignant condition in a patient. Methods are provided for detecting the presence of ovarian cancer in a sample, the absence of ovarian cancer in a sample, the stage of an ovarian cancer, the grade of an ovarian cancer, the benign or malignant nature of an ovarian cancer, the metastatic potential of an ovarian cancer, assessing the histological type of neoplasm associated with the ovarian cancer, the indolence or aggressiveness of the cancer, and other characteristics of ovarian cancer that are relevant to prevention, diagnosis, characterization, and therapy of ovarian cancer in a patient. Methods are also provided for assessing the efficacy of one or more test agents for inhibiting ovarian cancer, assessing the efficacy of a therapy for ovarian cancer, monitoring the progression of ovarian cancer, selecting an agent or therapy for inhibiting ovarian cancer, treating a patient afflicted with ovarian cancer, inhibiting ovarian cancer in a patient, and assessing the carcinogenic potential of a test compound.

### Glossary

The terms “sample”, “biological sample”, and the like, mean a material known or suspected of expressing or containing a plurality of kallikrien markers or polypeptides (2, 3, 4, 5, or 6 polypeptides), and optionally CA125 polypeptide, or polynucleotides encoding the polypeptides. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Nucleic acids and polypeptides may be isolated from the samples and utilized in the methods of the invention. In a preferred embodiment, the sample is a serum sample.

The term “subject” or “patient” refers to a warm-blooded animal such as a mammal, which is suspected of having ovarian cancer, or a condition, disease, or syndrome associated with ovarian cancer. Preferably, “subject” refers to a human.

“CA125”, “CA125 polypeptide”, or “carbohydrate antigen 125” refers to a high-molecular weight mucin, which can be defined by its ability to bind to monoclonal antibody OC125. The CA125 protein core comprises a short cytoplasmic core tail, a transmembrane domain, and a large and heavily glycosylated extracellular domain dominated by a repeat domain of 156 amino acids rich in serine, threonine, and proline (Yin B W and Lloyd K O, J Biol Chem. 2001, 276:27371-27375; O'Brian T J et al, Tumor Biol., 2001 22:348-366; and Hovig E. et al, Tumor Biol. 2001, 22:345-347). The sequence of CA125 is shown in GenBank Accession No. NP_{—}078966, AAL65133 and AF414442 (SEQ ID NO. 1). The term includes the native-sequence polypeptides, isoforms, precursors and chimeric polypeptides. The term also includes the native sequence polypeptide, including polypeptide variants and polypeptides with substantial sequence identity (e.g. at least about 45%, preferably 50%, 55%, 60%, 65%, 70%, 75%, 80%, 85%, 90%, 95%, 97%, 98%, or 99% sequence identity) to the sequence of GenBank Accession No.NP_{—}078966 (SEQ ID NO. 1), and that preferably retain the immunogenic activity of the corresponding native sequence polypeptide.

“Kallikrein polypeptides” or “kallikrein markers” comprise kallilkrein 5, kallikrein 6, kallikrein 7, kallikrein 8, kallikrein 10, and kallikrein 11. The term includes the native-sequence polypeptides, isoforms, precursors and chimeric polypeptides. The amino acid sequences for native kallik-rein polypeptides employed in the present invention include the sequences found in GenBank for each polypeptide as shown in Table 1, and in SEQ ID NO: 3 (kallilkrein 5), NO.6 (kallikrein 6), NO. 10 (kallikrein 7), NO. 13 (kallikrein 8), NO. 16 (kallikrein 10), and NOs. 19 and 20 (kallilkrein 11), or a portion thereof Other useful polypeptides are substantially identical to these sequences (e.g. at least about 45%, preferably 50%, 55%, 60%, 65%, 70%, 75%, 80%, 85%, 90%, 95%, 97%, 98%, or 99% sequence identity), and preferably retain the immunogenic activity of the corresponding native-sequence kallikrein polypeptide.

A “native-sequence polypeptide” comprises a polypeptide having the same amino acid sequence of a polypeptide derived from nature. Such native-sequence polypeptides can be isolated from nature or can be produced by recombinant or synthetic means.

The term “native-sequence polypeptide” specifically encompasses naturally occurring truncated or secreted forms of a polypeptide, polypeptide variants including naturally occurring variant forms (e.g., alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. in Table 1 and Accession No. NP_{—}078966, AF414442 and AAL65133 and shown in SEQ ID NOS: 1, 2, 3, 6, 10, 13, 16, 19 and 20. Such variants include, for instance, polypeptides wherein one or more amino acid residues are added to, or deleted from, the N— or C-terminus of the full-length or mature sequences of SEQ ID NOS: 1, 2, 3, 6, 10, 13, 16, 19 and 20, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an animo acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a CA125 or kallikrein polypeptide homolog, for example, the murine CA125 or kallikrein polypeptide.

Percent identity of two amino acid sequences, or of two nucleic acid sequences identified herein is defined as the percentage of amino acid residues or nucleotides in a candidate sequence that are identical with the amino acid residues in a CA125 or kallikrein polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

CA125 and kallikrien polypeptides include chimeric or fusion proteins. A “chimeric protein” or “fusion protein” comprises all or part (preferably biologically active) of a CA125 or kallikrein polypeptide operably linked to a heterologous polypeptide (i.e., a polypeptide other than the same CA125 or kallikrein polypeptide). Within the fusion protein, the term “operably linked” is intended to indicate that the CA125 or kallikrein polypeptide and the heterologous polypeptide are fused in-frame to each other. The heterologous polypeptide can be fused to the N-terminus or C-terminus of the CA125 or kallikrein polypeptide. A useful fusion protein is a GST fusion protein in which a kallikrein polypeptide is fused to the C-terminus of GST sequences. Another example of a fusion protein is an immunoglobulin fusion protein in which all or part of a CA125 or kallikrein polypeptide is fused to sequences derived from a member of the immunoglobulin protein family. Chimeric and fusion proteins can be produced by standard recombinant DNA techniques.

CA125 and kallikrein polypeptides may be isolated from a variety of sources, such as from human tissue types or from another source, or prepared by recombinant or synthetic methods, or by any combination of these and similar techniques.

“CA125 polynucleotides” or “polynucleotides encoding CA125” include nucleic acids that encode a native-sequence polypeptide, a polypeptide variant including a portion of a CA125 polypeptide, an isoform, precursor, and chimeric polypeptide. A nucleic acid sequence encoding native CA125 employed in the present invention includes the nucleic acid sequence in GenBank Accession No. AF414442 and SEQ ID NO. 2, or a fragment thereof

“Kallikrein polynucleotides” or “polynucleotides encoding kallikrein markers/polypeptides” refers to kallikrein 5 nucleic acids (KLK5), kallikrein 6 nucleic acids (KLK6), kallikrein 7 nucleic acids (KLK7), kallikrein 8 nucleic acids (KLK8), kallikrein 10 nucleic acids (KLK10), and/or kallikrein 11 nucleic acids (KLK11). The term includes nucleic acids that encode a native-sequence polypeptide, a polypeptide variant including a portion of a kallikrein polypeptide, an isoform, precursor, and chimeric polypeptide.

The polynucleotide sequences encoding native kallikrein polypeptides employed in the present invention include the nucleic acid sequences of the GenBank- Accession Nos. identified in Table 1, and in SEQ ID NOs: 4 and 5 (KLK5), NOs. 7, 8, and 9 (KLK6), NOs. 11 and 12 (KLK 7), NOs. 14 and 15 (KLK8), NOs. 17 and 18 (KLK10), and NOs. 21 and 22 (KLK 11), or a fragment thereof.

Polynucleotides encoding kallikrien polypeptides and CA125 include nucleic acid sequences complementary to these polynucleotides, and polynucleotides that are substantially identical to these sequences (e.g. at least about 45%, preferably 50%, 55%, 60%, 65%, 70%, 75%, 80%, 85%, 90%, 95%, 97%, 98%, or 99% sequence identity).

CA125 and kallikrein polynucleotides also include sequences which differ from a nucleic acid sequence of GenBank Accession Nos. identified in Table 1 and SEQ ID NOS: 2, 4, 5, 7, 8, 9, 11, 12, 14, 15, 17, 18, 21, and 22, due to degeneracy in the genetic code. As one example, DNA sequence polymorphisms within the nucleotide sequence of a CA125 or kallilcrein polypeptide may result in silent mutations which do not affect the amino acid sequence. Variations in one or more nucleotides may exist among individuals within a population due to natural allelic variation. DNA sequence polymorphisms may also occur which lead to changes in the amino acid sequence of CA125 or a kallikrein polypeptide.

CA125 and kallilrein polynucleotides also include nucleic acids that hybridize under stringent conditions, preferably high stringency conditions to a nucleic acid sequence of the GenBank Accession Nos. identified in Table 1 and SEQ ID NOS: 2, 4, 5, 7, 8, 9, 11, 12, 14, 15, 17, 18, 21, and 22. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0×sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

CA125 and kallikrein polynucleotides also include truncated nucleic acids or fragments and variant forms of the polynucleotides that arise by alternative splicing of an mRNA corresponding to a DNA.

The CA125 and kallikrien polynucleotides are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method.

A purality of kallikrein polypeptides or kallikrein polynucleotides are generally detected in the present invention. “Plurality” refers to 2, 3, 4, 5, or 6 kallikrein polypeptides or polynucleotides, in particular 3, 4, 5, or 6, preferably 4, 5, or 6, more preferably 5 or 6 kallikrein polypeptides or polynucleotides.

In an embodiment a plurality of kallikrein polypeptides is selected from the group consisting of kallikrein 5, kallikrein 7, and kallikrein S; kallikrein 5, kallikrein S, and kallikrein 10; kallikrein 7, kallikrein 8, and kallikrein 10; kallikrein 5, kallikrein 7, kallikrein 8, and kallikrein 10; kallikrein 7, kallikrein 8, kallikrein 10, and kallikrein 11; or kallikrein 5, kallikrein 7, kallikrein 8, kallikrein 10, and kallikrein 11. In another embodiment, a plurality of kallikrein polypeptides is selected from the group consisting of kallikrein 5, kallikrein 6, kallikrein 7, kallikrein 8, kallikrein 10 and kallikrein 11.

In an embodiment, a pluraity of kallikrein polynucleotides is selected from the group consisting of KLK5, KLK7, and KLK8; KLK5, KLK8 and KLK10; KLK7, KLK8 and KLK10; KLK5, KLK7, KLK8, and KLK10; KLK7, KLK8, KLK10 and KLK11, or KLK5, KLK7, KLK8, KLK10 and KLK11. In another embodiment, a plurality of kallikrein polynucleotides is selected from the group consisting of KLK5, KLK6, KLK7, KLK8, KLK10, and KLK11.

General Methods

A variety of methods can be employed for the diagnostic and prognostic evaluation of ovarian cancer involving kallikrein polypeptides, and optionally CA125 polypeptide, and polynucleotides encoding the polypeptides, and the identification of subjects with a predisposition to such disorders. Such methods may, for example, utilize polynucleotides encoding kallikrein polypeptides, and optionally CA125, and fragments thereof, and binding agents (e.g. antibodies aptamers) against kallikrein polypeptides, and optionally CA125 polypeptide, including peptide fragments. In particular, the polynucleotides and antibodies may be used, for example, for (1) the detection of either over- or under-expression of kallikrein polynucleotides, and optionally CA125, relative to a non-disorder state; and (2) the detection of either an over- or an under-abundance of kallikrein polypeptides, and optionally CA125, relative to a non-disorder state or the presence of modified (e.g., less than full length) kallikrein polypeptides, and optionally CA125, that correlate with a disorder state, or a progression toward a disorder state.

The invention also contemplates a method for detecting ovarian cancer comprising producing a profile of levels of a plurality of kallikrein markers, and optionally CA125, in cells from a patient, wherein the markers are kallikrein 5, kallikrein 6, kallikrein 7, kallikrein 8, kallikrein 10, and kallikrein 11, and comparing the profile with a reference to identify a protein profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring ovarian cancer by detecting a plurality of kallikrein polypeptides, and optionally CA125 polypeptide, or nucleic acids encoding the polypeptides in biological samples from a subject. These applications require that the amount of polypeptides or nucleic acids quantitated in a sample from a subject being tested be compared to a predetermined standard. The standard may correspond to levels quantitated for another sample or an earlier sample from the subject, or levels quantitated for a control sample. Levels for control samples from healthy subjects or ovarian cancer subjects may be established by prospective and/or retrospective statistical studies. Healthy or normal subjects who have no clinically evident disease or abnormalities may be selected for statistical studies. Diagnosis may be made by a finding of statistically different levels of a plurality of kallikrein polypeptides, and optionally CA125, or nucleic acids encoding same, compared to a control sample or previous levels quantitated for the same subject. A “significant difference” in levels of kallikrein markers or polynucleotides encoding the kallikrein markers in a patient sample compared to a control or standard (e.g. normal levels or levels in other samples from a patient) may represent levels that are higher or lower than the standard error of the detection assay, preferably the levels are at least about 1.5, 2, 3, 4, 5, or 6 times higher or lower, respectively, than the control or standard. The difference in levels of markers or polynucleotides may be a “statistically significant difference”

Nucleic Acid Methods/Assays

As noted herein an ovarian cancer may be detected based on the levels of polynucleoitdes encoding kallikrein polypeptides, and optionally CA125, in a sample. Techniques for detecting polynucleotides such as polymerase chain reaction (PCR) and hybridization assays are well known in the art.

Nucleotide probes for use in the detection of nucleic acid sequences in samples may be constructed using conventional methods known in the art. Suitable probes may be based on nucleic acid sequences encoding at least 5 sequential amino acids from regions of nucleic acids encoding kallikrein polypeptides, and optionally CA125, preferably they comprise 15 to 40 nucleotides. A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect polynucleoitides encoding kallikrein polypeptides, and optionally CA125, preferably in human cells. The nucleotide probes may also be useful in the diagnosis of ovarian cancer involving polynucleoitides encoding kallikrein polypeptides, and optionally CA125, in monitoring the progression of such disorder; or monitoring a therapeutic treatment.

Probes may be used in hybridization techniques to detect nucleic acids encoding a plurality of kallikrein polypeptides, and optionally CA125. The technique generally involves contacting and incubating nucleic acids (e.g. recombinant DNA molecules, cloned genes) obtained from a sample from a patient or other cellular source with probes under conditions favorable for the specific annealing of the probes to complementary sequences in the nucleic acids. After incubation, the non-annealed nucleic acids are removed, and the presence of nucleic acids that have hybridized to the probe if any are detected.

The detection of polynucleotides encoding kallikrein polypeptides and optionally CA125, may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, oligonucleotide primers may be employed in a PCR based assay to amplify a portion of nucleic acids encoding each of a plurality of kallikrein polypeptides, and optionally CA125, derived from a sample, wherein the oligonucleotide primers are specific for (i.e. hybridize to) polynucleotides encoding each of the plurality of kallikrein polypeptides, and optionally CA125. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of polynucleotides encoding a plurality of kallikrein polypeptides, and CA125. The primers and probes may be at least 10 nucleotides, and preferably at least 20 nucleotides in length. In an embodiment the primers and probes are at least about 1040 nucleotides in length.

Hybridization and amplification techniques described herein may be used to assay qualitative and quantitative aspects of expression of polynucleotides encoding kallikrein polypeptides, and optionally CA125. For example, RNA may be isolated from a cell type or tissue known to express these polynucleotides and tested utilizing the hybridization (e.g. standard Northern analyses) or PCR techniques referred to herein.

The primers and probes may be used in the above-described methods in situ i.e directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to sets of primers specifically designed against each of a plurality of kallikrein polynucleotide sequences, and optionally CA125. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected ovarian cancer and an individual who is not afflicted with ovarian cancer. The reaction may be performed on several dilutions of cDNA spanning at least two orders of magnitude. A statistically significant difference in expression in several dilutions of the subject sample as compared to the same dilutions of the non-cancerous sample may be considered positive for the presence of ovarian cancer.

Oligonucleotides or longer fragments derived from polynucleotides encoding each of a plurality of kallikrein polypeptides and optionally CA125, may be used as targets in a microarray. The microarray can be used to simultaneously monitor the expression levels of large numbers of genes. The information from the microarray may be used to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

Thus, the invention also includes an array comprising a plurality of polynucleotides encoding kallikrein marker(s), and optionally CA125 polynucleotides. The array can be used to assay expression of kallikrein polynucleotides, and optionally CA125 polynucleotides in the array. The invention allows the quantitation of expression of a plurality of kallikrein polynucleotides, and optionally CA125 polynucleotides.

In an embodiment, the array can be used to monitor the time course of expression of a plurality of kallikrein polynucleotides, and optionally CA125 polynucleotides, in the array. This can occur in various biological contexts such as tumor progression.

The array is also useful for ascertaining differential expression patterns of a plurality of kallikrein polynucleotides and optionally CA125 polynucleotides, in normal and abnormal cells. This provides a battery of polynucleotides that could serve as molecular targets for diagnosis or therapeutic intervention.

Protein Methods

Binding agents specific for a plurality of kallikrein markers and CA125 may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of an ovarian cancer in a subject may be determined by (a) contacting a sample from the subject with binding agents for a plurality of kallikrein polypeptides, and optionally CA125; (b) detecting in the sample levels of polypeptides that bind to the binding agents; and (c) comparing the levels of polypeptides with a predetermined standard or cut-off value.

“Binding agent” refers to a substance such as a polypeptide or antibody that specifically binds to a kallikrein or CA125 polypeptide. A substance “specifically binds” to a polypeptide if it reacts at a detectable level with the kallikrein or CA125 polypeptide, and does not react detectably with peptides containing unrelated sequences or sequences of different polypeptides. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises a kallikrein polypeptide or CA125 polypeptide sequence, a peptide variant thereof, or a non-peptide mimetic of~such a sequence. By way of example a kallikrein polypeptide sequence may be a peptide portion of a kallikrein polypeptide that is capable of modulating a function mediated by the kallikrein polypeptide.

An aptamer includes a DNA or RNA molecule that binds to polynucleotides and polypeptides. An aptamer that binds to a polypeptide (or binding domain) of a kallikrein polypeptide or a polynucleotide encoding a kallikrein polypeptide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

In certain other preferred embodiments, the binding agent is an antibody.

In an aspect the present invention provides a diagnostic method for monitoring or diagnosing ovarian cancer in a subject by quantitating a plurality of kallikrein polypeptides, and optionally CA125, in a biological sample from the subject comprising reacting the sample with antibodies specific for a plurality of kallikrein polypeptides, and optionally CA125, which are directly or indirectly labelled with detectable substances, and detecting the detectable substances.

In an aspect of the invention, a method for detecting ovarian cancer is provided comprising:
(a) obtaining a sample suspected of containing a plurality of kallikrein polypeptides, and optionally CA125, wherein the kallikrein polypeptides comprise or are selected from the group consisting of kallikrein 5, kallikrein 6, kallikrein 7, kallikrein 8, kallikrein 10 and kallikrein 11;
(b) contacting the sample with antibodies that specifically bind to the plurality of kallikrein polypeptides, and optionally CA125, under conditions effective to bind the antibodies and form complexes;
(c) measuring the amount of kallikrein polypeptides, and optionally CA125, present in the sample by quantitating the amount of the complexes; and
(d) comparing the amount of kallikrein polypeptides, and optionally CA125, present in the samples with the amount of polypeptides in a control, wherein a change or significant difference in the amount of polypeptides in the sample compared with the amount in the control is indicative of ovarian cancer.

In an embodiment, the invention contemplates a method for monitoring the progression of ovarian cancer in an individual, comprising:
(a) contacting antibodies which bind to each of a plurality of kallikrein polypeptides, and optionally CA125, with a sample from the individual so as to form binary complexes comprising each of the antibodies and polypeptides in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the stage and/or progression of the ovarian cancer in said individual.

The amount of complexes may also be compared to a value representative of the amount of the complexes from an individual not at risk of, or afflicted with, ovarian cancer at different stages.

Thus, antibodies specifically reactive with each of a plurality of kallikrein polypeptides, and CA125, or derivatives, such as enzyme conjugates or labeled derivatives, may be used to detect a plurality of kallikrein polypeptides, and optionally CA125, in various samples (e.g. biological materials). They may be used as diagnostic or prognostic reagents and they may be used to detect abnormalities in the levels of expression of a plurality of kallikrein polypeptides, and optionally CA125, or abnormalities in the structure, and/or temporal, tissue, cellular, or subcellular location of a plurality of kallikrein polypeptides, and optionally CA125. Antibodies may also be used to screen potentially therapeutic compounds in vitro to determine their effects on ovarian cancer involving a plurality of kallikrein polypeptides, and optionally CA125, and other conditions. In vitro immunoassays may also be used to assess or monitor the efficacy of particular therapies.

Antibodies may be used in any known immunoassays that rely on the binding interaction between antigenic determinants of a plurality of kallikrein polypeptides, and optionally CA125, and the antibodies. Examples of such assays are radioimmunoassays, enzyme immunoassays (e.g., ELISA), immunofluorescence, immunoprecipitation, latex agglutination, hemagglutination, and histochemical tests. These terms are well understood by those skilled in the art. A person skilled in the art will know, or can readily discern, other immunoassay formats without undue experimentation.

In particular, the antibodies may be used in immunohistochemical analyses, for example, at the cellular and sub-subcellular level, to detect a plurality of kallikrein polypeptides, and optionally CA125, to localize them to particular ovarian tumor cells and tissues, and to specific subcellular locations, and to quantitate the level of expression.

Antibodies for use in the present invention include monoclonal or polyclonal antibodies, immunologically active fragments (e.g. a Fab or (Fab)₂ fragments), antibody heavy chains, humanized antibodies, antibody light chains, genetically engineered single chain F, molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant kallikrein polypeptides or CA125 may be utilized to prepare antibodies. See, for example, Kohler et al. (1975) Nature 256:495-497; Kozbor et al. (1985) J. Immunol Methods 81:31-42; Cote et al. (1983) Proc Nat] Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. The antibodies specific for kallikrein polypeptides or CA125 used in the methods of the invention may also be obtained from scientific or commercial sources.

In an embodiment of the invention, antibodies are reactive against kallikrein polypeptides or CA125 if they bind with a K_{□} of greater than or equal to 10⁻⁷ M.

Antibodies that bind to kallikrein polypeptides or CA125 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol, enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against a kallikrein polypeptide or CA125. The second antibody may be labeled with a detectable substance to detect the primary antigen-antibody reaction. By way of example, if the antibody having specificity against a kallikrein polypeptide is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect a plurality of kallikrein polypeptides, and optionally CA125. Generally, antibodies may be labeled with detectable substances and kallikrein polypeptides, and optionally CA125, may be localised in tissues and cells based upon the presence of the detectable substance.

In the context of the methods of the invention, the sample, binding agents (e.g. antibodies) for a plurality of kallikrein polypeptides, and CA125 may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for a kallikrein polypeptide may be immobilized using sheep anti-mouse IgG Fc fragment specific antibody coated on the carrier or support.

Where radioactive labels are used as a detectable substance, a plurality of kallikrein polypeptides, and optionally CA125, may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

Therefore, in accordance with an embodiment of the invention, a method is provided wherein antibodies specific for each of a plurality of kallikrein polypeptides, and optionally CA125, are labelled with enzymes, substrates for the enzymes are added wherein the substrates are selected so that the substrates, or a reaction product of the enzymes and substrates, form fluorescent complexes with lanthanide metals. Lanthanide metals are added and the plurality of kallikrein polypeptides, and optionally CA125, are quantitated in the sample by measuring fluorescence of the fluorescent complexes. Antibodies specific for CA125 and each of a plurality of kallikrein polypeptides may be directly or indirectly labelled with enzymes. Enzymes are selected based on the ability of a substrate of the enzyme, or a reaction product of the enzyme and substrate, to complex with lanthanide metals such as europium and terbium. Examples of suitable enzymes include alkaline phosphatase and β-galactosidase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for a plurality of kallikrein polypeptides and CA125 may also be indirectly labelled with enzymes. For example, an antibody may be conjugated to one partner of a ligand binding pair, and the enzyme may be coupled to the other partner of the ligand binding pair: Representative examples include avidin-biotin, and riboflavin-riboflavin binding protein. In another embodiment, antibodies specific for the anti-kallikrein antibodies or anti- CA125 antibodies are labeled with an enzyme.

In accordance with an embodiment, the present invention provides means for determining a plurality of kallikrein polypeptides, and optionally CA125, in a sample, in particular a serum sample, by measuring a plurality of kallikrein polypeptides, and optionally CA125, by immunoassay. It will be evident to a skilled artisan that a variety of immunoassay methods can be used to measure a plurality of kallikrein polypeptides and CA125 in serum. In general, an immunoassay method may be competitive or noncompetitive. Competitive methods typically employ immobilized or immobilizable antibodies to each of a plurality of kallikrein polypeptides, and optionally CA125, and a labeled form of each of a plurality of kallikrein polypeptides, and optionally CA125. Kallikrein polypeptides and CA125 and labeled kallikrein polypeptides and CA125 compete for binding to anti-kallikrein antibodies and anti-CA125 antibodies. After separation of the resulting labeled kallikrein polypeptides and CA125 that have become bound to anti-kallikrein polypeptides and anti-CA125 (bound fraction) from that which has remained unbound (unbound fraction), the amount of the label in either bound or unbound fraction is measured and may be correlated with the amount of kallikrein polypeptides, and optionally CA125, in the test sample in any conventional manner, e.g., by comparison to a standard curve.

In an aspect, a non-competitive method is used for the determination of a plurality of kallikrein polypeptides, and optionally CA125, with the most common method being the “sandwich” method. In this assay, two types of antibodies specific for each of a plurality of kallikrein polypeptides, and optionally CA125 are employed. One type of antibody is directly or indirectly labeled (sometimes referred to as the “detection antibody”) and the other is immobilized or immobilizable (sometimes referred to as the “capture antibody”). The capture and detection antibodies can be contacted simultaneously or sequentially with a test sample. Sequential methods can be accomplished by incubating capture antibodies with the sample, and adding the detection antibodies at a predetermined time thereafter (sometimes referred to as the “forward” method); or the detection antibodies can be incubated with the sample first and then the capture antibodies added (sometimes referred to as the “reverse” method). After the necessary incubation(s) have occurred, to complete the assay, the capture antibodies are separated from the liquid test mixture, and labels are measured in at least a portion of the separated capture antibody phase or the remainder of the liquid test mixture. Generally the labels are measured in the capture antibody phase since it comprises kallikrein polypeptides, and optionally CA125, bound by (“sandwiched” between) the capture and detection antibodies. In an embodiment, the label may be measured without separating the capture antibodies and liquid test mixture.

In a typical two-site immunometric assay for a plurality of kallikrein polypeptides, and optionally CA125, one or both of the capture and detection antibodies are polygonal antibodies or one or both of the capture and detection antibodies are monoclonal antibodies (i.e. polyclonal/polyclonal, monoclonal/monoclonal, or monoclonal/polyclonal). The labels used with the detection antibodies can be selected from any of those known conventionally in the art. The labels may be an enzyme or a chemiluminescent moiety, but it can also be a radioactive isotope, a fluorophor, a detectable ligand (e.g., detectable by a secondary binding by a labeled binding partner for the ligand), and the like. Preferably antibodies are labelled with enzymes which are detected by adding substrates that are selected so that a reaction product of the enzymes and substrates forms fluorescent complexes. Capture antibodies may be selected so that they provide a means for being separated from the remainder of the test mixture. Accordingly, the capture antibodies can be introduced to the assay in an already immobilized or insoluble form, or can be in an immobilizable form, that is, a form which enables immobilization to be accomplished subsequent to introduction of the capture antibodies to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, a capture antibody may be immobilized using a species specific antibody for the capture antibody that is bound to the solid phase.

A particular sandwich immunoassay method of the invention employs two types of antibodies, first antibodies are reactive against each of a plurality of kallikrein polypeptides, and optionally CA125, and second antibodies having specificity against antibodies reactive against each of a plurality of kallikrein polypeptides, and optionally CA125, labelled with enzymatic labels, and fluorogenic substrates for the enzymes. An enzyme may be alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+ fluorescence in a time-resolved mode. Fluorescence intensity is measured using a time-resolved fluorometer as described herein.

The above-described immunoassay methods and formats are intended to be exemplary and are not limiting.

Computer Systems

Computer readable media comprising a plurality of kallikrein markers, and optionally CA125, is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on a plurality of kallikrein markers, and optionally CA125.

A variety of data processor programs and formats can be used to store information on a plurality of kallikrein markers, and optionally CA125, on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has ovarian cancer or a pre-disposition to ovarian cancer, comprising determining the presence or absence of a plurality of kallikrein markers, optionally CA125, and/or polynucleotides encoding same, and based on the presence or absence of the plurality of kallikrein markers, optionally CA125, and/or polynucleotides encoding same, determining whether the patient has ovarian cancer or a pre-disposition to ovarian cancer, and optionally recommending treatment for the ovarian cancer or pre-ovarian cancer condition.

The invention also provides in an electronic system and/or in a network, a method for determining whether a subject has ovarian cancer or a pre-disposition to ovarian cancer associated with a plurality of kallikrein markers, and optionally CA125, and/or polynucleotides encoding same, comprising determining the presence or absence of a plurality of kallikrein markers, and optionally CA125, and/or polynucleotides encoding same, and based on the presence or absence of the plurality of kallikrein markers, and optionally CA125, and/or polynucleotides encoding same, determining whether the subject has ovarian cancer or a pre-disposition to ovarian cancer, and optionally recommending treatment for the ovarian cancer or pre-ovarian cancer condition.

The invention further provides in a network, a method for determining whether a subject has ovarian cancer or a pre-disposition to ovarian cancer associated with a plurality of kallikrein markers, optionally CA125 and/or polynucleotides encoding same, comprising: (a) receiving phenotypic information on the subject and information on a plurality of kallikrein markers, optionally CA125 and/or polynucleotides encoding same associated with samples from the subject; (b) acquiring information from the network corresponding to the plurality of kallikrein markers, optionally CA125, and/or polynucleotides encoding same; and (c) based on the phenotypic information and information on the plurality of kallikrein markers, optionally CA125, and/or polynucleotides encoding same, determining whether the subject has ovarian cancer or a pre-disposition to ovarian cancer; and (d) optionally recommending treatment for the ovarian cancer or pre-ovarian cancer condition.

The invention still further provides a system for identifying selected records that identify an ovarian cancer cell. A system of the invention generally comprises a digital computer; a database server coupled to the computer; a database coupled to the database server having data stored therein, the data comprising records of data comprising a plurality of kallikrein markers, optionally CA125, and/or polynucleotides encoding same, and a code mechanism for applying queries based upon a desired selection criteria to the data file in the database to produce reports of records which match the desired selection criteria.

In an aspect of the invention a method is provided for detecting an ovarian cancer cell using a computer having a processor, memory, display, and input/output devices, the method comprising the steps of:
(a) creating records of a plurality of kallikrein markers, optionally CA125, and/or polynucleotides encoding same, isolated from a sample suspected of containing an ovarian cancer cell;
(b) providing a database comprising records of data comprising a plurality of kallikrein markers, optionally CA125, wherein the markers are kallikrein 5, kallikrein 6, kallikrein 7, kallikrein 8, kallikrein 10, and kallikrein 11, and/or comprising polynucleotides encoding same; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from a cell that is an ovarian cancer cell.

The invention contemplates a business method for determining whether a subject has ovarian cancer or a pre-disposition to ovarian cancer associated with a plurality of kallikrcin markers, optionally CA125, and/or polynucleotides encoding same, comprising: (a) receiving phenotypic information on the subject and information on a plurality of kallikrein markers, optionally CA125, and/or polynucleotides encoding same, associated with samples from the subject; (b) acquiring information from a network corresponding to the plurality of kallikrein markers, optionally CA125, and/or polynucleotides encoding same; and (c) based on the phenotypic information, information on a plurality of kallikrein markers, optionally CA125, and/or polynucleotides encoding same, and acquired information, determining whether the subject has ovarian cancer or a pre-disposition to ovarian cancer; and (d) optionally recommending treatment for the ovarian cancer or pre-ovarian cancer condition.

Imaging Methods

Antibodies specific for each of a plurality of kallikrein polypeptides, and optionally CA125, may also be used in imaging methodologies in the management of ovarian cancer. The invention provides a method for imaging tumors associated with a plurality of kallikrein polypeptides, and optionally CA125.

In an embodiment the method is an iii vivo method and a subject or patient is administered imaging agents that carry imaging labels and are capable of targeting or binding to each of a plurality of kallikrein polypeptides, and optionally CA125. In the method each imaging agent is labeled so that it can be distinguished during the imaging. The imaging agents are allowed to incubate in vivo and bind to the plurality of kallikrein polypeptides, and optionally CA125, associated with ovarian tumors. The presence of label is localized to the ovarian cancer, and the localized label is detected using imaging devices known to those skilled in the art.

The imaging agents may be antibodies or chemical entities that recognize the plurality of kallikrein polypeptides, and optionally CA125. In an aspect of the invention an imaging agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for kallikrein polypeptides and CA125 used in the methods of the invention may be obtained from scientific or commercial sources, or isolated native or recombinant kallikrein and CA125 polypeptides may be utilized to prepare antibodies etc as described herein.

An imaging agent may be a peptide that mimics the epitope for an antibody specific for kallikrein polypeptide or CA125 and binds to kallikrein polypeptide or CA125. The peptide may be produced on a commercial synthesizer using conventional solid phase chemistry. By way of example, a peptide may be prepared that includes either tyrosine, lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a tumor producing a plurality of kallikrein polypeptides, and optionally CA125.

Imaging agents carry labels to image the plurality of kallikrein polypeptides and CA125. Agents may be labelled for use in radionuclide imaging. In particular, agents may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{191m-191}Os, ¹⁰⁹Pd, ³²P, ⁴²K, ²²⁶Ra, ¹⁸⁶Re, ¹⁸⁵Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ²¹³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹TI ⁷⁷Br, or ¹⁸F, and it is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). lodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m} Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for 1 In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An imaging agent may also be labeled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) J ACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on ill vivo nuclear magnetic resonance imaging.)

In the case of radiolabeled agents, the agents may be administered to the patient, localized to the tumor having a plurality of kallikrein polypeptides, and optionally CA125, with which the agents bind, and detected or “imaged” in vivo using known techniques such as radionuclear scanning using, for example, a gamma camera or emission tomography. [See for example, A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for a plurality of kallikrein polypeptides, and optionally CA125, or fragments thereof having the same epitope specificity, are bound to a suitable radioisotope, or a combination thereof, and administered parenterally. For ovarian cancer, administration preferably is intravenous. The bio-distribution of the labels can be monitored by scintigraphy, and accumulations of the labels can be related to the presence of ovarian cancer cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see, U.S. Pat. No. 4,732,864). These agents are useful in diagnosis, staging and visualization of cancer, in particular ovarian cancer, so that surgical and/or radiation treatment protocols can be used more efficiently.

Screening Methods

The invention also contemplates methods for evaluating test agents or compounds for their ability to inhibit ovarian cancer or potentially contribute to ovarian cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)2, and Fab expression library fragments, and epitope-binding fragments thereof)), nucleic acids (e.g. antisense, interference RNA) and small organic or inorganic molecules. The agents or compounds may be endogenous physiological compounds or natural or synthetic compounds.

The invention also provides a method for assessing the potential efficacy of a test agent for inhibiting ovarian cancer in a patient, the method comprising comparing:
(a) levels of a plurality of kallikrein markers, optionally CA125, and/or polynucleotides encoding same, in a first sample obtained from a patient and exposed to the test agent, wherein the markers comprise or are selected from the group consisting of kallikrein 5, kallikrein 6, kallikrein 7, kallikrein 8, kallikrein 10, and kallikrein 11, and
(b) levels of the plurality of kallikrein markers, optionally CA125, and/or polynucleotides encoding same, in a second sample obtained from the patient, wherein the sample is not exposed to the test agent, wherein a significant difference in the levels of expression of a plurality of kallikrein markers, optionally CA125, and/or polynucleotides encoding same, in the first sample, relative to the second sample, is an indication that the test agent is potentially efficacious for inhibiting ovarian cancer in the patient.

The first and second samples may be portions of a single sample obtained from a patient or portions of pooled samples obtained from a patient.

In an aspect, the invention provides a method of selecting an agent for inhibiting ovarian cancer in a patient comprising:
(a) obtaining a sample comprising cancer cells from the patient;
(b) separately maintaining aliquots of the sample in the presence of a plurality of test agents;
(c) comparing a plurality of kallikrein markers, optionally CA125, and/or polynucleotides encoding same, in each of the aliquots, wherein the markers comprise or are selected from the group consisting of kallikrein 5, kallikrein 6, kallikrein 7, kallikrein 8, kallikrein 10, and kallikrein 11; and
(d) selecting one of the test agents which alters the levels of the kallikrein markers, optionally CA125, and/or polynucleotides encoding same, in the aliquot containing that test agent relative to other test agents.

Still another aspect of the present invention provides a method of conducting a drug discovery business comprising:
(a) providing one or more methods or assay systems for identifying agents that inhibit ovarian cancer in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the ovarian carcinogenic potential of a test compound comprising:
(a) maintaining separate aliquots of ovarian cells in the presence and absence of the test compound; and
(b) comparing a plurality of kallikrein markers, optionally CA125, and/or polynucleotides encoding same, in each of the aliquots, wherein the markers comprise or are selected from the group consisting of kallikrein 5, kallikrein 6, kallikrein 7, kallikrein 8, kallikrein 10, and kallikrein 11.

A significant difference between the levels of the markers in the aliquot maintained in the presence of (or exposed to) the test compound relative to the aliquot maintained in the absence of the test compound, indicates that the test compound possesses ovarian carcinogenic potential.

Kits

The methods described herein may be performed by utilizing pre-packaged diagnostic kits comprising at least a plurality of kallikrein nucleic acids or binding agents (e.g. antibodies) or CA125 nucleic acids or binding agents described herein, which may be conveniently used, e.g., in clinical settings, to screen and diagnose patients, and to screen and identify those individuals afflicted with or exhibiting a predisposition to ovarian cancer.

Thus, the invention also contemplates kits for carrying out the methods of the invention. Such kits typically comprise two or more components required for performing a diagnostic assay. Components include but are not limited to compounds, reagents, containers, and/or equipment.

In an embodiment, a container with a kit comprises binding agents as described herein. By way of example, the kit may contain antibodies specific for a plurality of kallikrein polypeptides, and optionally CA125, antibodies against the antibodies labelled with enzymes; and substrates for the enzymes. The kit may also contain microtiter plate wells, standards, assay diluent, wash buffer, adhesive plate covers, and/or instructions for carrying out a method of the invention using the kit.

In an aspect of the invention, the kit includes antibodies or antibody fragments which bind specifically to epitopes of each of a plurality of kallikrein polypeptides, and optionally CA125, and means for detecting binding of the antibodies to epitopes associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains radiolabelled antibody preparations for in vivo imaging.

A kit may be designed to detect the level of polynucleotides encoding kallikrein polypeptides, and optionally CA125 polynucleotides, in a sample. Such kits generally comprise oligonucleotide probes or primers, as described herein, that hybridize to a plurality of polynucleotides encoding kallikrein polypeptides and optionally CA125. Such oligonucleotides may be used, for example, within a PCR or hybridization procedure. Additional components that may be present within the kits include second oligonucleotides and/or diagnostic reagents to facilitate detection of a plurality polynucleotides encoding kallikrein polypeptides, and optionally CA125 polynucleotides.

The reagents suitable for applying the screening methods of the invention to evaluate compounds may be packaged into convenient kits described herein providing the necessary materials packaged into suitable containers.

Applications

Kallikrein polypeptides (in particular, kallikrein 5, 6, 10 and 11), optionally in combination with CA125, are targets for ovarian cancer immunotherapy. Such immunotherapeutic methods include the use of antibody therapy, in vivo vaccines, and ex vivo immunotherapy approaches.

In one aspect, the invention provides antibodies specific for a plurality of kallikrein polypeptides (for example, kallikreins 5, 6, 10 and 11) and optionally CA125, that may be used systemically to treat ovarian cancer. Preferably antibodies are used that target the tumor cells but not the surrounding non-tumor cells and tissue. Thus, the invention provides a method of treating a patient susceptible to, or having a cancer that expresses a plurality of kallikrein polypeptides, and optionally CA125, comprising administering to the patient an effective amount of antibodies that bind specifically to a plurality of kallikrein polypeptides, and optionally CA125. In another aspect, the invention provides a method of inhibiting the growth of tumor cells expressing a plurality of kallikrein polypeptides, and optionally CA125, comprising administering to a patient antibodies which bind specifically to the plurality of kallikrein polypeptides, and optionally CA125, in amounts effective to inhibit growth of the tumor cells. Antibodies specific for a plurality of kallikrein polypeptides, and optionally CA125, may also be used in a method for selectively inhibiting the growth of, or killing a cell expressing a plurality of kallikrein polypeptides, and optionally CA125, comprising reacting antibody immunoconjugates or immunotoxins with the cell in an amount sufficient to inhibit the growth of, or kill the cell.

By way of example, unconjugated antibodies specific for a plurality of kallikrein polypeptides, and optionally CA125, may be introduced into a patient such that the antibodies bind to cancer cells expressing a plurality of kallikrein polypeptides, and optionally CA125, and mediate growth inhibition of such cells (including the destruction thereof), and the tumor, by mechanisms which may include complement-mediated cytolysis, antibody-dependent cellular cytotoxicity, altering the physiologic function of a plurality of kallikrein polypeptides, and optionally CA125, and/or the inhibition of ligand binding or signal transduction pathways. In addition to unconjugated antibodies, antibodies specific for a plurality of kallikrein polypeptides, and optionally CA125, conjugated to therapeutic agents (e.g. immunoconjugates) may also be used therapeutically to deliver the agents directly to tumor cells expressing a plurality of kallikrein polypeptides, and optionally CA125, and thereby destroy the tumor. Examples of such agents include abrin, ricin A, Pseudonmonas exotoxin, or diphtheria toxin, proteins such as tumor necrosis factor, alpha-interferon, beta-interferon, nerve growth factor, platelet derived growth factor, tissue plasminogen activator, and biological response modifiers such as lymphokines, interleukin-1, interleukin-2, interleukin-6, granulocyte macrophage colony stimulating factor, granulocyte colony stimulating factor, or other growth factors.

Cancer immunotherapy using antibodies specific for a plurality of kallikrein polypeptides, and optionally CA125, may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of a method of the invention, antibodies specific for a plurality of kallikrein polypeptides, optionally in combination with antibodies specific for CA125, capable of inhibiting the growth of cancer cells expressing a plurality of kallikrein polypeptides, and optionally CA125, are administered in a therapeutically effective amount to cancer patients whose tumors express or overexpress a plurality of kallikrein polypeptides, and optionally CA125. The invention may provide a specific, effective and long-needed treatment for ovarian cancer. The antibody therapy methods of the invention may be combined with other therapies including chemotherapy and radiation.

Patients may be evaluated for the presence and levels of a plurality of kallikrein polypeptides, and optionally CA125, expression and overexpression in tumors, preferably using immunohistochemical assessments of tumor tissue, quantitative imaging as described herein, or other techniques capable of reliably indicating the presence and degree of expression of a plurality of kallikrein polypeptides, and optionally CA125. Immunohistochemical analysis of tumor biopsies or surgical specimens may be employed for this purpose.

Antibodies specific for a plurality of kallikrein polypeptides and CA125 useful in treating cancer include those that are capable of initiating a potent immune response against the tumor and those that are capable of direct cytotoxicity. In this regard, the antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, antibodies specific for a plurality of kallikrein polypeptides and CA125 that exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a combination of antibodies specific for a plurality of kallikrein polypeptides and optionally CA125, may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of combinations, or “cocktails” of different individual antibodies recognizing epitopes of a plurality of kallikrein polypeptides, and optionally CA125. Such cocktails may have certain advantages inasmuch as they contain antibodies that bind to different epitopes and/or exploit different effector mechanisms or combine directly cytotoxic antibodies with antibodies that rely on immune effector functionality. Such antibodies in combination may exhibit synergistic therapeutic effects. In addition, the administration of the antibodies may be combined with other therapeutic agents, including but not limited to chemotherapeutic agents, androgen-blockers, and immune modulators (e.g., IL2, GM-CSF). The antibodies may be administered in their “naked” or unconjugated form, or may have therapeutic agents conjugated to them.

The antibodies specific for a plurality of kallikrein polypeptides and optionally CA125, used in the practice of the method of the invention may be formulated into pharmaceutical compositions comprising a carrier suitable for the desired delivery method. Suitable carriers include any material which when combined with the antibodies retains the anti-tumor function of the antibodies and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of expression of a plurality of kallikrein polypeptides, and optionally CA125, in the patient, the extent of circulating kallikrein polypeptide antigens, and optionally CA125 antigens, the desired steady-state antibody concentration level, frequency of treatment, and the influence of any chemotherapeutic agents used in combination with a treatment method of the invention.

Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of antibodies necessary to be therapeutically effective in a particular context. Repeated administrations may be required to achieve tumor inhibition or regression. Direct administration of antibodies specific for a plurality of kallikrein polypeptides and optionally CA125 is also possible and may have advantages in certain situations.

Patients may be evaluated for a plurality of kallikrein polypeptides and optionally CA125, preferably in serum, in order to assist in the determination of the most effective dosing regimen and related factors. The assay methods described herein, or similar assays, may be used for quantitating circulating kallikrein polypeptide and optionally CA125 levels in patients prior to treatment. Such assays may also be used for monitoring throughout therapy, and may be useful to gauge therapeutic success in combination with evaluating other parameters, such as serum kallikrein polypeptides, and optionally CA125, levels.

The invention further provides vaccines formulated to contain a plurality of kallikrein polypeptides, and optionally CA125, or fragments thereof The use in anti-cancer therapy of tumor antigens in a vaccine for generating humoral and cell-mediated immunity is well known and, for example, has been employed in prostate cancer using human PSMA and rodent PAP immunogens (Hodge et al., 1995, Int. J. Cancer 63: 231-237; Fong et al., 1997, J. Immunol. 159: 3113-3117). These methods can be practiced by employing a plurality of kallikrein polypeptides, and optionally CA125, or fragments thereof, or nucleic acids and recombinant vectors capable of expressing and appropriately presenting the kallikrein and optionally CA125, immunogens.

By way of example, viral gene delivery systems may be used to deliver nucleic acids encoding a plurality of kallikrein polypeptides, and optionally CA125. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding a plurality of kallikrein polypeptides, and optionally CA125, or fragments thereof introduced into the patient (e.g., intramuscularly) to induce an anti-tumor response.

Various ex vivo strategies may also be employed. One approach involves the use of cells to present kallikrein and optionally CA125 antigens to a patient's immune system. For example, autologous dendritic cells which express MHC class I and II, may be pulsed with a plurality of kallikrein polypeptides, and optionally CA125, or peptides thereof that are capable of binding to MHC molecules, to thereby stimulate ovarian cancer patients' immune systems (See, for example, Tjoa et al., 1996, Prostate 28: 65-69; Murphy et al., 1996, Prostate 29: 371-380).

Anti-idiotypic antibodies specific for a plurality of kallilarein polypeptides, and optionally CA125, can also be used in anti-cancer therapy as a vaccine for inducing an immune response to cells expressing the polypeptides. The generation of anti-idiotypic antibodies is well known in the art and can readily be adapted to generate anti-idiotypic antibodies that mimic an epitope on a kallikrein polypeptide or CA125 (see, for example, Wagner et al., 1997, Hybridoma 16: 33-40; Foon et al., 1995, J Clin Invest 96: 334342; Herlyn et al., 1996, Cancer Immunol Immunother 43: 65-76). Such antibodies can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing a plurality of kallikrein polypeptides, and optionally CA125. Constructs comprising DNA encoding kallikrein and optionally CA125 polypeptides/immunogens and appropriate regulatory sequences may be injected directly into muscle or skin of an individual, such that the cells of the muscle or skin take-up the construct and express the encoded kallikrein or CA125 polypeptides/immunogens. The polypeptides/immunogens may be expressed as cell surface proteins or be secreted. Expression of the polypeptides/immunogens results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation or propagation) of a cell expressing a plurality of kallikrein polypeptides, and optionally CA125. This method comprises reacting immunoconjugates of the invention (e.g., a heterogeneous or homogenous mixture) with the cell so that the kallikrein polypeptides, and optionally CA125, form complexes with the immunoconjugates. A subject with a neoplastic or preneoplastic condition can be treated when the inhibition of cellular activity results in cell death.

In another aspect, the invention provides methods for selectively inhibiting a cell expressing a plurality of kallikrein polypeptides, and optionally CA125, by reacting a combination of immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver nucleic acids encoding a plurality of kallikrein polypeptides, and optionally CA125, to a targeted organ, tissue, or cell population. Methods well known to those skilled in the art may be used to construct recombinant vectors that will express antisense nucleic acid molecules for kallikrein polypeptides and CA125. (See, for example, the techniques described in Sambrook et al (supra) and Ausubel et al (supra)).

Genes encoding a plurality of kallikrein polypeptides, and optionally CA125, can be turned off by transfecting a cell or tissue with vectors that express high levels of a desired kallikrein or CA125 polypeptide-encoding fragments. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of genes encoding kallikrein polypeptides, and optionally CA125, i.e., the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a plurality of kallikrein polypeptides, and optionally CA125.

Specific ribozyme cleavage sites within any potential RNA target may initially be identified by scanning the target molecule for ribozyme cleavage sites which include the following sequences, GUA, GUTU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Kallikrein polypeptides, optionally CA125 polypeptide, and/or polynucleotides encoding the polypeptides, and fragments thereof, antibodies and/or agents identified using a method of the invention, or combinations thereof, may be used in the treatment of ovarian cancer or diseases, conditions or syndromes associated with ovarian cancer, in a subject. A combination of kallikrein polypeptides and/or polynucleotides encoding the kallikreins (e.g. kallikreins 7 and 8) and inhibitors (antisense, antibodies, or agents) of other kallikreins (e.g. kallikreins 5, 6, 10 and 11) and/or CA125 may be used in a prognostic or therapeutic method of the invention. The polypeptides, polynucleotides, and agents may be formulated into compositions for administration to subjects suffering from ovarian cancer. Therefore, the present invention also relates to a composition comprising a plurality of kallikrein polypeptides and optionally CA125, or nucleic acids encoding the polypeptides, or a fragment thereof, or an agent identified using a method of the invention, and a pharmaceutically, acceptable carrier, excipient or diluent. A method for treating or preventing ovarian cancer in a subject is also provided comprising administering to a patient in need thereof, a plurality of kallikrein polypeptides and optionally CA125, or nucleic acids encoding the polypeptides, an agent identified in accordance with a method of the invention, and/or a composition of the invention.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). The compositions of the invention may be administered concurrently, separately, or sequentially with other therapeutic agents or therapies.

The following non-limiting examples are illustrative of the present invention:

### EXAMPLE 1

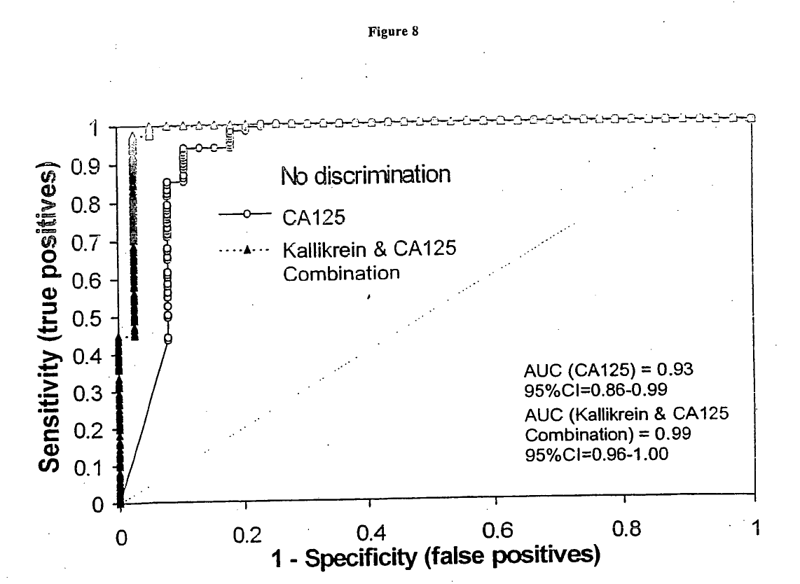

To investigate the additional discriminatory value of the kallikreins to CA125 a logistic regression model was developed. Included in the study were serum samples from.39 ovarian cancer patients and 194 non-cancer females. The age of the patients was as follows: median=59, range 32-82. The age of the controls was as follows: median=46; range=22-77. The model was adjusted for the following variables: f(x)=−0.29 hK5*+0.12*hK6−0.65*hK7−0.6*hK8+1.09*hK10+0.98*hK11+0.057*CA125−0.62. For these data, the crude odds ratio and the 95% confidence interval were found to be 2.71 and 1.91−3.84 (p<0.001). The log likelihood scores for this multivariate logistic regression model, which incorporated the combined variables for each patient was calculated. From these data, by picking different thresholds for the regression function values, a ROC curve was devised which shows the added value of using kallikreins and CA125 together in a multivariate function.(AUC, 0.99;95%CI,0.96−1.00). (See FIG. 8.) Statistically significant correlations between age and other studied variables were not observed.

### EXAMPLE 2

Statistically significant differences in serum kallikrein concentration was found between patient and control subjects for kallikreins hK5 (p<0.0001), hK7 (p=0.007), hK8 (p=0.005), hK10 (p=0.0003) and CA125 (p<0.0001) by the Mann-Whitney test. The diagnostic sensitivity (SENS) and specificity (SPEC) for each one of these markers were as follows (SENS/SPEC; both as %): 31/95 (hK5); 62/71 (hK7); 62/70 (hK10); 54/54 (hK11); 89/94 (CA125). When these data were combined in a logistic regression model, kallikreins 5 and 10 did not contribute to a great extent to the sensitivity and specificity of CA125. The area under the curve of CA125 alone (93%) improved by a further 1% when adding hK6, by 2% when adding hK11, 3% when adding hK7 and 5% when adding hK8. The combination of CA125 and hK8 resulted in an AUC of 98%.

Below is a summary of each marker and its ability to separate the cases and controls.
hK5: high values associated with cancer test+ is hK5>0.10, test− is hK5<=0.10 sensitivity=31%, specificity=95%,
AUC=0.62, p(AUC)=0.02
Wilcoxon rank sum test has p<0.0001.
Of the 233 persons analyzed, 207 have value zero for hK5 (27 cases, 180 controls).
Possible good marker
hK6: high values associated with cancer test+ is hK6>6.3, test− is hK6<=6.3. sensitivity=69%, specificity=40%,
AUC=0.50, p(AUC)=1.00
Wilcoxon rank sum test has p=0.91.
Not a good marker
hK7: low values associated with cancer test+ is hK7<2.05, test− is hK7>=2.05 sensitivity=62%, specificity=71%,
AUG=0.64, p(AUC)=0.006
Wilcoxon rank sum test has p=0.007.
Possible good marker
hK8: low values associated with cancer test+ is hk8<13.0, test− is hK8>=13.0 sensitivity=72%, specificity=42%,
AUC=0.64, p(AUC)=0.006
Wilcoxon rank sum test has p=0.005
Possible good marker
hK10: high values associated with cancer test+ is hK10>1.42, test− is hK10<=1.42 sensitivity=62%, specificity=70%,
AUC=0.68, p(AUC)=0.0004
Wilcoxon rank sum test has p=0.0003.
Best single kallikrein marker
hK11: high values associated with cancer test+ is hK11>0.14, test− is hK11<=0.14 sensitivity=54%, specificity=54%,
AUC=0.58, p(AUC)=0.12
Wilcoxon rank sum test has p=0.11.
Not a good marker
CA125: high values associated with cancer test+ is Ca125>34, test− is Ca125<=34 sensitivity=89%, specificity=94%,
AUC=0.933, p(AUC)=<0.0001
Wilcoxon rank sum test has p<0.0001.
Good marker
   After some further multivariate analysis of only the kallikrein markers, the combination of hK 7, 8, 10 and 11 was a preferred set. This combination was arrived at by looking at the incremental AUC as markers were combined. Below is a summary of all the models tried:
hK10 alone, AUC=0.68
hK10+hK7: AUC0.88
hK10+hK7+hK8: AUC=0.90
hK10+hK7+hK8+hK11: AUC=0.925
Multivariate model of hK7, hK8, hK10, hK11, call it hK7_{—}8_{—}10_{—}11
hK7_{—}8_{—}110_{—}11:
Calculate SA=2.00−1.49(hK7)−0.34(hK8)+1.16(hK10)+3.50(hK11) high values associated with cancer test+ is SA>−1.15, test− is SA<=−1.15 sensitivity=87%, specificity=89%
AUC=0.93, p(AUC)=<0.0001
Wilcoxon rank sum test has p<0.0001.
Good marker
The hK marker that added the most to CA125 was also investigated.
CA125 alone, AUC=0.933
CA125+hK8: AUC=0.978
Multivariate model of Ca125, hK8, call it Ca 125_hK8
Ca125_hK8:
SC=−1.71+0.086(Ca125)−0.47(hK8). high values associated with cancer test+ is SC>−2.52, test− is SC<−2.52 sensitivity=97%, specificity=90%,
AUC=0.978, p(AUC)=<0.0001.
Wilcoxon rank sum test has p<0.0001.
Good marker
   Below is a summary of the above analyses:
   a) The preferred kallikrien marker alone is hK10, AUC=0.68
   b) CA125 has an AUC of 0.933
   c) The preferred combination of kallikrein markers increases the AUC up to 0.925, which is close to the CA125 AUC of0.933
   d) Adding a kallikrein marker to CA125 can increase the AUC up to 0.978

How does CA125 alone compare with the multivariate kallikrein model hK7_{—}8_{—}10_{—}11?(based on 39 cases and 186 controls evaluated with CA125)

| | Sensitivity | Specificity | misclassification |
|---|---|---|---|
| CA125 | 90% | 94% | 12FP, 4FN, total |
| | | | 16 pts misclassified |
| hK7_8_10_11 | 85% | 89% | 31FP, 4FN, total |
| | | | 35 pts misclassified |
| both positive | 77% | 100% | 0FP, 9FN, total |
| | | | 9 pts misclassified |
| either positive | 97% | 82% | 33FP, 1FN, total |
| | | | 34 pts misclassified |

How does CA125 alone compare with the multivariate model of CA125 plus hK8? (based on 39 cases and 186 controls evaluated with CA125)

| | Sensitivity | Specificity | misclassification |
|---|---|---|---|
| CA125 | 90% | 94% | 12FP, 4FN, total |
| | | | 16 pts misclassified |
| CA125_hK8 | 95% | 91% | 17FP, 2FN, total |
| | | | 19 pts misclassified |

Kallikrein markers approach CA125 in terms of AUC and sensitivity, but their specificity is not as high. Adding hK8 to CA125 improves sensitivity but specificity is lower than CA125 alone.

Summary

a) The best kallikrein marker alone is hK10, area under the ROC curve (AUC)=0.68.
b) CA125 has an AUC of 0.933. Adding a single kallikrein marker to CA125 can get the AUC up to 0.978. Adding hK8 to CA125 improves sensitivity but specificity is lower compared with CA125 alone.
c) The best combination of kallikrein markers gets the AUC up to 0.925, which is close to the CA125 AUC of 0.933. Kallikrein markers approach CA125 in terms of AUC and sensitivity, but their specificity is lower.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Below full citations are set out for the references referred to in the specification.

TABLE 1

| | Kallikrein Nucleic Acid | |
|---|---|---|
| Kallikrein Polypeptide | Designation | GenBank Accession No. |
| Kallikrein 5 | KLK5 | AAD26429, AF135028, AF168768 |
| Kallikrein 6 | KLK6 | AAB66483, AF013988 (CDS 174 . . . 881), AF149289 |
| | | (CDS join 3567 . . . 3606, 4346 . . . 4502, 8122 . . . 8369, |
| | | 9791 . . . 9927, 11805 . . . 11957) U62801 (CDS 246 . . . 980) |
| Kallikrein 7 | KLK7 | AAC37551, L33404 (CDS 16 . . . 777), AF166330 |
| | | (CDS join 3237 . . . 3309, 3722 . . . 3869, 4566 . . . 4813, |
| | | 5129 . . . 5265, 7362 . . . 7517) (mRNA join(1756 . . . 1785, |
| | | 3179 . . . 3309, 3722 . . . 3869, 4566 . . . 4813, 5129 . . . 5265, |
| | | 7362 . . . 8265)/product = “stratum corneum |
| | | chymotryptic enzyme”/note = “alternatively |
| | | spliced”; mRNA join (1756 . . . 1785, 3179 . . . 3309, |
| | | 3722 . . . 3869, 4566 . . . 4813, 5129 . . . 5265, 7362 . . . 7991)/note = |
| | | “alternatively spliced”; mRNA join |
| | | (1821 . . . 1864, 3179 . . . 3309, 3722 . . . 3869, 4566 . . . 4813, |
| | | 5129 . . . 5265, 7362 . . . 8265)/product = “stratum |
| | | corneum chymotryptic enzyme”/note = “alternatively spliced”; mRNA join |
| | | (1821 . . . 1864, 3179 . . . 3309, 3722 . . . 3869, 4566 . . . 4813, |
| | | 5129 . . . 5265, 7362 . . . 7991)/note = “alternatively spliced” |
| Kallikrein 8 | KLK8 | BAA28673, AB009849 (CDS 35 . . . 817), AF095743 (CDS join |
| | | 1035 . . . 1104, 1619 . . . 1778, 1944 . . . 2206, 4304 . . . 4437, |
| | | 5974 . . . 6129, mRNA 500 . . . 670, 1027 . . . 1104, 1619 . . . 1778, |
| | | 1944 . . . 2206, 4304 . . . 4437, 5974 . . . 6174), AB010780 |
| | | (CDS join 1 . . . 39, 418 . . . 712, 878 . . . >946), AF055982 |
| Kallikrein 10 | KLK10 | AAC14266, AF055481 (CDS join 614 . . . 701, 2455 . . . 2635, |
| | | 3589 . . . 3863, 4195 . . . 4328, 4793 . . . 4945, mRNA join |
| | | 48 . . . 120, 605 . . . 701, 2455 . . . 2635, 3589 . . . 3863, |
| | | 4195 . . . 4328, 4793 . . . 5474), NM_002776 (CDS 220 . . . 1050) |
| Kallikrein 11 | KLK11 | BAA33404, AAD47815, AB012917 (CDS 26 . . . 874), AF164623 |
| | | (CDS 4224 . . . 4263, 5061 . . . 5217, 5545 . . . 5810, |
| | | 6627 . . . 6763, 7158 . . . 7310) (mRNA join (2313 . . . 2398, |
| | | 4189 . . . 4263, 5061 . . . 5217, 5545 . . . 5810, 6627 . . . 6763, 7158 . . . 7622) |

TABLE 2
Descriptive statistics for hk5, hk6, hk7, hk8, hk10 and hk11 serum
protein levels in controls and patients with ovarian cancer

| | Mean | Standard Error | Median | Range | p value* |
|---|---|---|---|---|---|
| hk5(ng/ml) | | | | | |
| Non cancer (N = 194) | 0.063 | 0.029 | 0.00 | 0.00-4.50 | |
| Cancer (N = 39) | 0.48 | 0.18 | 0.00 | 0.00-5.70 | |
| % Increase** | 661% | — | — | — | <0.001 |
| hk6(ng/ml) | | | | | |
| Non cancer (N = 194) | 6.96 | 0.18 | 6.60 | 1.60-15.30 | |
| Cancer (N = 39) | 9.88 | 2.20 | 6.60 | 1.50-70.80 | |
| % Increase** | 42% | — | — | — | 0.91 |
| hk7(ng/ml) | | | | | |
| Non cancer (N = 194) | 2.60 | 0.071 | 2.67 | 0.30-6.00 | |
| Cancer (N = 39) | 2.49 | 0.41 | 1.80 | 0.00-10.80 | |
| % Decrease** | 4% | — | 33% | — | 0.007 |
| hk8(ng/ml) | | | | | |
| Non cancer (N = 194) | 11.74 | 0.27 | 11.70 | 2.40-22.20 | |
| Cancer (N = 39) | 11.91 | 1.88 | 6.90 | 0.00-46.20 | |
| % Decrease** | — | — | 41% | — | 0.005 |
| hK10(ng/ml) | | | | | |
| Non cancer (N = 194) | 1.16 | 0.051 | 1.08 | 0.00-4.20 | |
| Cancer (N = 39) | 6.51 | 2.46 | 1.59 | 0.27-90.0 | |
| % Increase** | 461% | — | 40% | — | <0.001 |
| hK11(ng/ml) | | | | | |
| Non cancer (N = 194) | 0.21 | 0.018 | 0.12 | 00-1.30 | |
| Cancer (N = 39) | 0.79 | 0.21 | 0.18 | 0.00-5.52 | |
| % Increase** | 276% | — | 50% | | 0.011 |

*Calculated by the Mann Whitney test
**Calculated by assuming that value in non-cancerouos tissue is 100%

TABLE 3
Correlations between the studied
variables in 194 non-cancer cases

| variable | | hK5 | hK6 | hK7 | hK8 | hK10 | hK11 | CA125 |
|---|---|---|---|---|---|---|---|---|
| hK5 | rₛ | 1.000 | 0.034 | −0.053 | 0.066 | 0.134 | 0.150 | 0.101 |
| | p | . | 0.642 | 0.462 | 0.359 | 0.062 | 0.037 | 0.172 |
| hK6 | rₛ | 0.034 | 1.000 | 0.114 | 0.298 | 0.191 | 0.120 | −0.160 |
| | p | 0.642 | . | 0.115 | 0.000 | 0.008 | 0.097 | 0.029 |
| hK7 | rₛ | −0.053 | 0.114 | 1.000 | 0.497 | 0.321 | 0.399 | 0.135 |
| | p | 0.462 | 0.115 | . | 0.000 | 0.000 | 0.000 | 0.066 |
| hK8 | rₛ | 0.066 | 0.298 | 0.497 | 1.000 | 0.263 | 0.396 | 0.048 |
| | p | 0.359 | 0.000 | 0.000 | . | 0.000 | 0.000 | 0.519 |
| hK10 | rₛ | 0.134 | 0.191 | 0.321 | 0.263 | 1.000 | 0.176 | 0.035 |
| | p | 0.062 | 0.008 | 0.000 | 0.000 | . | 0.014 | 0.638 |
| hK11 | rₛ | 0.150 | 0.120 | 0.399 | 0.396 | 0.176 | 1.000 | 0.125 |
| | p | 0.037 | 0.097 | 0.000 | 0.000 | 0.014 | . | 0.090 |
| CA125 | rₛ | 0.101 | −0.160 | 0.135 | 0.048 | 0.035 | 0.125 | 1.000 |
| | p | 0.172 | 0.029 | 0.066 | 0.519 | 0.638 | 0.090 | . |

TABLE 4
Correlations between the studied variables
in 39 ovarian cancer cases

| variable | | hK5 | hK6 | hK7 | hK8 | hK10 | hK11 | CA125 |
|---|---|---|---|---|---|---|---|---|
| hK5 | rₛ | 1.000 | 0.475 | 0.553 | 0.554 | 0.618 | 0.584 | 0.507 |
| | p | . | 0.002 | 0.000 | 0.000 | 0.000 | 0.000 | 0.001 |
| hK6 | rₛ | 0.475 | 1.000 | 0.327 | 0.513 | 0.470 | 0.661 | 0.530 |
| | p | 0.002 | . | 0.042 | 0.001 | 0.003 | 0.000 | 0.001 |
| hK7 | rₛ | 0.553 | 0.327 | 1.000 | 0.695 | 0.690 | 0.748 | 0.262 |
| | p | 0.000 | 0.042 | . | 0.000 | 0.000 | 0.000 | 0.107 |
| hK8 | rₛ | 0.554 | 0.513 | 0.695 | 1.000 | 0.602 | 0.783 | 0.443 |
| | p | 0.000 | 0.001 | 0.000 | . | 0.000 | 0.000 | 0.005 |
| hK10 | rₛ | 0.618 | 0.470 | 0.690 | 0.602 | 1.000 | 0.706 | 0.548 |
| | p | 0.000 | 0.003 | 0.000 | 0.000 | . | 0.000 | 0.000 |
| hK11 | rₛ | 0.584 | 0.661 | 0.748 | 0.783 | 0.706 | 1.000 | 0.556 |
| | p | 0.000 | 0.000 | 0.000 | 0.000 | 0.000 | . | 0.000 |
| CA125 | rₛ | 0.507 | 0.530 | 0.262 | 0.443 | 0.548 | 0.556 | 1.000 |
| | p | 0.001 | 0.001 | 0.107 | 0.005 | 0.000 | 0.000 | . |

TABLE 5

| | | | CANCER | | | | | | CONTROL | | | | |
|---|---|---|---|---|---|---|---|---|---|---|---|---|---|
| | n | mean | median | sd | min | max | n | mean | median | sd | min | max | p(mean) |
| hK5 | 39 | 0.48 | 0.00 | 1.14 | 0.00 | 5.70 | 194 | 0.06 | 0.00 | 0.40 | 0.00 | 4.50 | 0.03 |
| hK6 | 39 | 9.88 | 6.60 | 13.77 | 1.50 | 70.80 | 194 | 6.97 | 6.60 | 2.60 | 1.60 | 15.30 | 0.20 |
| hK7 | 39 | 2.49 | 1.80 | 2.54 | 0.00 | 10.60 | 194 | 2.60 | 2.67 | 0.99 | 0.30 | 6.00 | 0.79 |
| hK8 | 39 | 11.91 | 6.90 | 11.80 | 0.00 | 46.20 | 194 | 11.74 | 11.70 | 3.80 | 2.40 | 22.20 | 0.93 |
| hK10 | 39 | 6.52 | 1.59 | 15.40 | 0.27 | 90.00 | 194 | 1.17 | 1.08 | 0.72 | 0.00 | 4.20 | 0.04 |
| hK11 | 39 | 0.60 | 0.16 | 1.36 | 0.00 | 5.52 | 194 | 0.21 | 0.12 | 0.26 | 0.00 | 1.30 | 0.01 |
| CA125 | 39 | 049 | 277 | 971 | 20 | 4494 | 186 | 24 | 21 | 9 | 0 | 84 | 0.00 |
| hK7_8_10_11 | 39 | 4.51 | 0.50 | 15.0 | −4.74 | 92.26 | 194 | −3.84 | −3.68 | 2.07 | −10.05 | 1.07 | 0.002 |
| CA125_hK8 | 39 | 48.64 | 15.02 | 02.86 | −5.86 | 382.85 | 186 | −5.07 | −4.92 | 1.91 | −9.49 | 0.50 | 0.0002 |

| | Wilcoxon | | | | | CUT- | SENSI- | SPECI- | |
|---|---|---|---|---|---|---|---|---|---|
| | p(median) | p(logistic) | AUC (se) | | p(AUC) | POINT | TIVITY | FICITY | |
| hK5 | <.0001 | 0.005 | 0.62 | (.05) | 0.0200 | >0.10 | 31% | 95% | hK5 |
| hK6 | 0.9100 | 0.0570 | 0.50 | (.05) | 1.0000 | >6.30 | 69% | 40% | hK6 |
| hK7 | 0.0070 | 0.6500 | 0.64 | (.05) | 0.0060 | <2.05 | 62% | 71% | hK7 |
| hK8 | 0.0050 | 0.8700 | 0.64 | (.05) | 0.0060 | <13.0 | 72% | 42% | hK8 |
| hK10 | 0.0003 | 0.0002 | 0.68 | (.05) | 0.0004 | >1.42 | 62% | 70% | hK10 |
| hK11 | 0.1100 | 0.0008 | 0.58 | (.05) | 0.1200 | >0.14 | 54% | 54% | hK11 |
| CA125 | <.0001 | <.0001 | 0.933 | (.028) | <.0001 | >34 | 90% | 94% | CA125 |
| hK7_8_10_11 | <.0001 | <.001 | 0.925 | (.030) | <.0001 | >−1.32 | 85% | 88% | hK7_8_10_11 |
| CA125_hK8 | <.0001 | <.001 | 0.978 | (.017) | <.0001 | >−2.52 | 97% | 90% | CA125_hK8 |

1. A method for detecting a plurality of kallikrein markers associated with ovarian cancer in a patient comprising: (a) obtaining a sample from a patient; (b) detecting in the sample a plurality of kallikrein markers and optionally CA125, wherein the kallikrein markers comprise or are selected from the group consisting of kallikrein 5, kallikrein 6, kallikrein 7, kallikrein 8, kallikrein 10, and kallikrein 11; and (c) comparing the detected amounts with amounts detected for a standard.

2. A method for diagnosing and monitoring ovarian cancer in a subject comprising detecting in a sample from the subject a plurality of kallikrein markers, wherein the kallikrein markers comprise or are selected from the group consisting of kallikrein 5, kallikrein 6, kallikrein 7, kallikrein 8, kallikrein 10, and kallikrein.

3. A method as claimed in claim 1 wherein the plurality of kallikrein markers are detected using antibodies that bind to each of the plurality of kallikrein markers or parts thereof.

4. A method as claimed in claim 1 which further comprises detecting CA125.

5. A method of detecting ovarian cancer in a patient, the method comprising comparing: (a) levels of a plurality of kallikrein markers, and optionally CA125, in a sample from the patient, wherein the kallikrein markers comprise or are selected from the group consisting of kallikrein 5, kallikrein 6, kallikrein 7, kallikrein 8, kallikrein 10, and kallikrein 11; and (b) normal levels of expression of the plurality of kallikrein markers, and optionally CA125, in a control sample, wherein a significant difference in levels of kallikrein markers and optionally CA125, relative to the corresponding normal levels, is indicative of ovarian cancer.

6. A method for monitoring the progression of ovarian cancer in a patient, the method comprising: (a) detecting in a sample from the patient at a first time point, a plurality of kallikrein markers, wherein the kallikrein markers comprise or are selected from the group consisting of kallikrein 5, kallikrein 6, kallikrein 7, kallikrein 8, kallikrein 10, and kallikrein 11; (b) repeating step (a) at a subsequent point in time; and (c) comparing levels detected in steps (a) and (b), and thereby monitoring the progression of ovarian cancer.

7. A method for determining in a patient whether ovarian cancer has metastasized or is likely to metastasize in the future, the method comprising comparing (a) levels of a plurality of kallikrein markers, and optionally CA125, in a patient sample, wherein the kallikrein markers comprise or are selected from the group consisting of kallikrein 5, kallikrein 6, kallikrein 7, kallikrein 8, kallikrein 10, and kallikrein 11; and (b) normal levels or non-metastatic levels of the kallikrein markers and optionally CA125, in a control sample wherein a significant difference between the levels of expression in the patient sample and the normal levels or non-metastatic levels is an indication that the ovarian cancer has metastasized.

8. A method for assessing the aggressiveness or indolence of ovarian cancer comprising comparing: (a) levels of expression of a plurality of kallikrein markers, and optionally CA125, in a patient sample, wherein the kallikrein markers comprise or are selected from the group consisting of kallikrein 5, kallikrein 6, kallikrein 7, kallikrein 8, kallikrein 10, and kallikrein 11; and (b) normal levels of expression of the plurality of markers and optionally CA125, in a control sample, wherein a significant difference between the levels in the patient sample and normal levels is an indication that the cancer is aggressive or indolent.

9. A method for diagnosing and monitoring ovarian cancer in a sample from a subject comprising isolating nucleic acids from the sample; and detecting in the sample polynucleotides encoding a plurality of kallikrein markers, and optionally CA125, wherein the kallikrein markers comprise or are selected from the group consisting of Icallikrein 5, kallikrein 6, kallikrein 7, kallikrein 8, kallikrein 10, and kallikrein.

10. A method as claimed in claim 9 wherein significant differences in the levels of the polynucleotides in the sample compared to a control is indicative of disease, disease stage, and/or prognosis.

11. A method for determining the presence or absence of ovarian cancer in a subject comprising: (a) contacting a sample obtained from the subject with oligonucleotides that hybridize to polynucleotides encoding kallikrein markers, and optionally CA125, wherein the kallikrein markers comprise or are selected from the group consisting of kallikrein 5, kallikrein 6, kallikrein 7, kallikrein 8, kallikrein 10, and kallikrein 11; and (b) detecting in the sample a level of nucleic acids in the sample that hybridize to the polynucleotides relative to a predetermined cut-off value, and therefrom determining the presence or absence of ovarian cancer in the subject.

12. A method as claimed in claim 11, wherein the nucleic acids are mRNA and the levels of nucleic acids are detected by polymerase chain reaction.

13. A method as claimed in claim 11 wherein the nucleic acids are mRNA and the amounts of mRNA are detected using a hybridization technique, employing oligonucleotide probes that hybridize to kallikrein markers, and optionally CA125.

14. A method for assessing the potential efficacy of a test agent for inhibiting ovarian cancer in a patient, the method comprising comparing: (a) levels of a plurality of kallilcrein markers, optionally CA125, and/or polynucleotides encoding same, in a first sample obtained from a patient and exposed to the test agent, wherein the kallikrein markers comprise or are selected from the group consisting of kallikrein 5, kallikrein 6, kallikrein 7, kallilcrein 8, kallikrein 10, and kallikrein 11, and (b) levels of the plurality of kallikrein markers, optionally CA125, and/or polynucleotides encoding same, in a second sample obtained from the patient, wherein the sample is not exposed to the test agent, wherein a significant difference in the levels of expression of the plurality of kallikrein markers, optionally CA125, and/or polynucleotides encoding same, in the first sample, relative to the second sample, is an indication that the test agent is potentially efficacious for inhibiting ovarian cancer in the patient.

15. A method of claim 14 wherein the first and second samples are portions of a single sample obtained from the patient.

16. A method of claim 14 wherein the first and second samples are portions of pooled samples obtained from the patient.

17. A method of assessing the efficacy of a therapy for inhibiting ovarian cancer in a patient, the method comprising comparing: (a) levels of a plurality of kallikrein markers, optionally CA125, and/or polynucleotides encoding same, in a first sample obtained from the patient, wherein the kallikrein markers comprise or are selected from the group consisting of kallikrein 5, kallikrein 6, kallikrein 7, kallikrein 8, kallikrein 10, and kallikrein 11, and (b) levels of the kallikrein markers, optionally CA125, and/or polynucleotides encoding same, in a second sample obtained from the patient following therapy, wherein a significant difference in the levels of expression of the kallikrein markers, optionally CA125, and/or polynucleotides encoding same, in the second sample, relative to the first sample, is an indication that the therapy is efficacious for inhibiting ovarian cancer in the patient.

18. A method of selecting an agent for inhibiting ovarian cancer in a patient the method comprising (a) obtaining a sample comprising cancer cells from the patient; (b) separately exposing aliquots of the sample in the presence of a plurality of test agents; (c) comparing levels of a plurality of kallikrein markers, optionally CA125, and/or polynucleotides encoding same, in each of the aliquots, wherein the kallikrein markers comprise or are selected from the group consisting of kallikrein 5, kallikrein 6, kallikrein 7, kallikrein 8, kallikrein 10, and kallikrein 11; and (d) selecting one of the test agents which alters the levels of kallikrein markers, optionally CA125, and/or polynucleotides encoding same, in the aliquot containing that test agent, relative to other test agents.

19. A method of inhibiting ovarian cancer in a patient, the method comprising (a) obtaining a sample comprising cancer cells from the patient; (b) separately maintaining aliquots of the sample in the presence of a plurality of test agents; (c) comparing levels of a plurality of kallikrein markers, optionally CA125, and/or polynucleotides encoding same, in each of the aliquots, wherein the kallikrein markers comprise or are selected from the group consisting of kallikrein 5, kallikrein 6, kallikrein 7, kallikrein 8, kallikrein 10, and kallikrein 11; and (d) administering to the patient at least one of the test agents which alters the levels of kallikrein markers, optionally CA125, and/or polynucleotides encoding same, in the aliquot containing that test agent, relative to other test agents.

20. A method of assessing the ovarian cell carcinogenic potential of a test compound, the method comprising: (a) maintaining separate aliquots of ovarian cells in the presence and absence of the test compound; and (b) comparing expression of a plurality of markers, optionally CA125, and/or polynucleotides encoding same, in each of the aliquots, wherein the markers comprise or are selected from the group consisting of kallikrein 5, kallikrein 6, kallikrein 7, kallikrein 8, kallikrein 10, and kallikrein 11, and wherein a significant difference in levels of kallikrein markers, optionally CA125, and/or polynucleotides encoding same, in the aliquot maintained in the presence of the test compound, relative to the aliquot maintained in the absence of the test compound, is an indication that the test compound possesses ovarian cell carcinogenic potential.

21. A method of inhibiting ovarian cancer in a patient at risk for developing ovarian cancer, the method comprising inhibiting expression of genes encoding kallikrein markers and optionally CA125, wherein the kallikrein markers comprise or are selected from the group consisting of kallikrein 5, kallikrein 6, kallikrein 7, kallikrein 8, kallikrein 10, and kallikrein 11.

22. A method of claim 1 wherein the plurality comprises at least three of the markers.

23. A method of claim 1 wherein the plurality comprises at least five of the markers.

24. A method of claim 1 wherein the plurality of kallikrein markers is selected from the group consisting of kallikrein 5, kallikrein 7, and kallikrein 8; kallikrein 5, kallikrein 8, and kallikrein 10; kallikrein 7, kallikrein 8, and kallikrein 10; kallikrein 5, kallikrein 7, kallikrein 8, and kallikrein 10; kallikrein 7, kallikrein 8, kallikrein 10, and kallikrein 11; kallikrein 5, kallikrein 7, kallikrein 8, kallikrein 10, and kallikrein 11; or kallikrein 5, kallikrein 6, kallikrein 7, kallikrein 8, kallikrein 10 and kallikrein 11.

25. A method of any proceeding claims wherein the kallikrein markers are kallikrein 7, kallikrein 8, kallikrein 10 and kallirkein 11.

26. A method of claim 1 wherein the patient sample comprises serum obtained from the patient.

27. A kit for carrying out a method as claimed in claim 1.

28. A kit for assessing whether a patient is afflicted with ovarian cancer, the kit comprising reagents that specifically bind with a plurality of kallikrein markers and optionally CA125, wherein the kallikrein markers comprise or are selected from the group consisting of kallikrein 5, kallikrein 6, kallikrein 7, kallikrein 8, kallikrein 10, and kallikrein 11.

29. A kit for assessing the suitability of each of a plurality of agents for inhibiting ovarian cancer in a patient, the kit comprising: (a) the plurality of agents; and (b) reagents for detecting a plurality of kallikrein markers and optionally CA125, wherein the kallikrein markers comprise or are selected from the group consisting of kallikrein 5, kallikrein 6, kallikrein 7, kallikrein 8, kallikrein 10, and kallikrein 11.

30. A kit as claimed in claim 28 wherein the reagents are antibodies that specifically bind with protein or protein fragments corresponding to kallikrein markers and optionally CA125.


SEQUENCE LISTING (22 sequences)

SEQ ID NO: 1 - protein, 22152 aa, Homo sapiens
  misc_feature (13877)..(13878): Xaa can be any naturally occurring amino acid
  misc_feature (13880)..(13880): Xaa can be any naturally occurring amino acid
  misc_feature (13887)..(13887): Xaa can be any naturally occurring amino acid
  misc_feature (13890)..(13891): Xaa can be any naturally occurring amino acid
  misc_feature (13893)..(13893): Xaa can be any naturally occurring amino acid
  misc_feature (13903)..(13903): Xaa can be any naturally occurring amino acid
  misc_feature (13913)..(13914): Xaa can be any naturally occurring amino acid
  misc_feature (13916)..(13916): Xaa can be any naturally occurring amino acid
  misc_feature (13928)..(13929): Xaa can be any naturally occurring amino acid
  misc_feature (13938)..(13938): Xaa can be any naturally occurring amino acid
  misc_feature (13940)..(13941): Xaa can be any naturally occurring amino acid
  misc_feature (14569)..(14571): Xaa can be any naturally occurring amino acid
  misc_feature (14575)..(14575): Xaa can be any naturally occurring amino acid
  misc_feature (14579)..(14579): Xaa can be any naturally occurring amino acid
  misc_feature (14581)..(14581): Xaa can be any naturally occurring amino acid
  misc_feature (14587)..(14591): Xaa can be any naturally occurring amino acid
  misc_feature (14593)..(14594): Xaa can be any naturally occurring amino acid
  misc_feature (14725)..(14727): Xaa can be any naturally occurring amino acid
  misc_feature (14731)..(14731): Xaa can be any naturally occurring amino acid
  misc_feature (14735)..(14735): Xaa can be any naturally occurring amino acid
  misc_feature (14737)..(14737): Xaa can be any naturally occurring amino acid
  misc_feature (14743)..(14747): Xaa can be any naturally occurring amino acid
  misc_feature (14749)..(14750): Xaa can be any naturally occurring amino acid
  misc_feature (15661)..(15663): Xaa can be any naturally occurring amino acid
  misc_feature (15667)..(15667): Xaa can be any naturally occurring amino acid
  misc_feature (15671)..(15671): Xaa can be any naturally occurring amino acid
  misc_feature (15673)..(15673): Xaa can be any naturally occurring amino acid
  misc_feature (15679)..(15683): Xaa can be any naturally occurring amino acid
  misc_feature (15685)..(15686): Xaa can be any naturally occurring amino acid
  misc_feature (15972)..(15974): Xaa can be any naturally occurring amino acid
  misc_feature (15978)..(15978): Xaa can be any naturally occurring amino acid
  misc_feature (15982)..(15982): Xaa can be any naturally occurring amino acid
  misc_feature (15984)..(15984): Xaa can be any naturally occurring amino acid
  misc_feature (15990)..(15994): Xaa can be any naturally occurring amino acid
  misc_feature (15996)..(15997): Xaa can be any naturally occurring amino acid
  misc_feature (16008)..(16008): Xaa can be any naturally occurring amino acid
  misc_feature (16015)..(16015): Xaa can be any naturally occurring amino acid
  misc_feature (16017)..(16017): Xaa can be any naturally occurring amino acid
  misc_feature (16021)..(16021): Xaa can be any naturally occurring amino acid
  misc_feature (16025)..(16025): Xaa can be any naturally occurring amino acid
  misc_feature (16034)..(16034): Xaa can be any naturally occurring amino acid
  misc_feature (16037)..(16037): Xaa can be any naturally occurring amino acid
  misc_feature (16040)..(16040): Xaa can be any naturally occurring amino acid
  misc_feature (16046)..(16046): Xaa can be any naturally occurring amino acid
  misc_feature (16051)..(16051): Xaa can be any naturally occurring amino acid
  misc_feature (16053)..(16055): Xaa can be any naturally occurring amino acid
  misc_feature (16058)..(16058): Xaa can be any naturally occurring amino acid
  misc_feature (16062)..(16063): Xaa can be any naturally occurring amino acid
  misc_feature (16065)..(16065): Xaa can be any naturally occurring amino acid
  misc_feature (16072)..(16072): Xaa can be any naturally occurring amino acid
  misc_feature (16075)..(16076): Xaa can be any naturally occurring amino acid
  misc_feature (16078)..(16078): Xaa can be any naturally occurring amino acid
  misc_feature (16088)..(16088): Xaa can be any naturally occurring amino acid
  misc_feature (16268)..(16269): Xaa can be any naturally occurring amino acid
  misc_feature (16278)..(16278): Xaa can be any naturally occurring amino acid
  misc_feature (16280)..(16281): Xaa can be any naturally occurring amino acid
  misc_feature (16373)..(16374): Xaa can be any naturally occurring amino acid
  misc_feature (16376)..(16376): Xaa can be any naturally occurring amino acid
  misc_feature (16383)..(16383): Xaa can be any naturally occurring amino acid
  misc_feature (16386)..(16387): Xaa can be any naturally occurring amino acid
  misc_feature (16389)..(16389): Xaa can be any naturally occurring amino acid
  misc_feature (16399)..(16399): Xaa can be any naturally occurring amino acid
  misc_feature (16409)..(16410): Xaa can be any naturally occurring amino acid
  misc_feature (16412)..(16412): Xaa can be any naturally occurring amino acid
  misc_feature (16424)..(16425): Xaa can be any naturally occurring amino acid
  misc_feature (16434)..(16434): Xaa can be any naturally occurring amino acid
  misc_feature (16436)..(16437): Xaa can be any naturally occurring amino acid
  misc_feature (16439)..(16441): Xaa can be any naturally occurring amino acid
  misc_feature (16445)..(16445): Xaa can be any naturally occurring amino acid
  misc_feature (16449)..(16449): Xaa can be any naturally occurring amino acid
  misc_feature (16451)..(16451): Xaa can be any naturally occurring amino acid
  misc_feature (16457)..(16461): Xaa can be any naturally occurring amino acid
  misc_feature (16463)..(16464): Xaa can be any naturally occurring amino acid
  misc_feature (16841)..(16842): Xaa can be any naturally occurring amino acid
  misc_feature (16844)..(16844): Xaa can be any naturally occurring amino acid
  misc_feature (16851)..(16851): Xaa can be any naturally occurring amino acid
  misc_feature (16854)..(16855): Xaa can be any naturally occurring amino acid
  misc_feature (16857)..(16857): Xaa can be any naturally occurring amino acid
  misc_feature (16867)..(16867): Xaa can be any naturally occurring amino acid
  misc_feature (16877)..(16878): Xaa can be any naturally occurring amino acid
  misc_feature (16880)..(16880): Xaa can be any naturally occurring amino acid
  misc_feature (16892)..(16893): Xaa can be any naturally occurring amino acid
  misc_feature (16902)..(16902): Xaa can be any naturally occurring amino acid
  misc_feature (16904)..(16905): Xaa can be any naturally occurring amino acid
  misc_feature (16907)..(16909): Xaa can be any naturally occurring amino acid
  misc_feature (16913)..(16913): Xaa can be any naturally occurring amino acid
  misc_feature (16917)..(16917): Xaa can be any naturally occurring amino acid
  misc_feature (16919)..(16919): Xaa can be any naturally occurring amino acid
  misc_feature (16925)..(16929): Xaa can be any naturally occurring amino acid
  misc_feature (16931)..(16932): Xaa can be any naturally occurring amino acid
  misc_feature (17465)..(17466): Xaa can be any naturally occurring amino acid
  misc_feature (17468)..(17468): Xaa can be any naturally occurring amino acid
  misc_feature (17475)..(17475): Xaa can be any naturally occurring amino acid
  misc_feature (17478)..(17479): Xaa can be any naturally occurring amino acid
  misc_feature (17481)..(17481): Xaa can be any naturally occurring amino acid
  misc_feature (17491)..(17491): Xaa can be any naturally occurring amino acid
  misc_feature (17501)..(17502): Xaa can be any naturally occurring amino acid
  misc_feature (17504)..(17504): Xaa can be any naturally occurring amino acid
  misc_feature (17516)..(17517): Xaa can be any naturally occurring amino acid
  misc_feature (17526)..(17526): Xaa can be any naturally occurring amino acid
  misc_feature (17528)..(17529): Xaa can be any naturally occurring amino acid
  misc_feature (17531)..(17533): Xaa can be any naturally occurring amino acid
  misc_feature (17537)..(17537): Xaa can be any naturally occurring amino acid
  misc_feature (17541)..(17541): Xaa can be any naturally occurring amino acid
  misc_feature (17543)..(17543): Xaa can be any naturally occurring amino acid
  misc_feature (17549)..(17553): Xaa can be any naturally occurring amino acid
  misc_feature (17555)..(17556): Xaa can be any naturally occurring amino acid
  misc_feature (17567)..(17567): Xaa can be any naturally occurring amino acid
  misc_feature (17574)..(17574): Xaa can be any naturally occurring amino acid
  misc_feature (17576)..(17576): Xaa can be any naturally occurring amino acid
  misc_feature (17580)..(17580): Xaa can be any naturally occurring amino acid
  misc_feature (17584)..(17584): Xaa can be any naturally occurring amino acid
  misc_feature (17593)..(17593): Xaa can be any naturally occurring amino acid
  misc_feature (17596)..(17596): Xaa can be any naturally occurring amino acid
  misc_feature (17599)..(17599): Xaa can be any naturally occurring amino acid
  misc_feature (17605)..(17605): Xaa can be any naturally occurring amino acid
  misc_feature (17610)..(17610): Xaa can be any naturally occurring amino acid
  misc_feature (17612)..(17614): Xaa can be any naturally occurring amino acid
  misc_feature (17617)..(17617): Xaa can be any naturally occurring amino acid
  misc_feature (17621)..(17622): Xaa can be any naturally occurring amino acid
  misc_feature (17624)..(17624): Xaa can be any naturally occurring amino acid
  misc_feature (17631)..(17631): Xaa can be any naturally occurring amino acid
  misc_feature (17777)..(17778): Xaa can be any naturally occurring amino acid
  misc_feature (17780)..(17780): Xaa can be any naturally occurring amino acid
  misc_feature (17787)..(17787): Xaa can be any naturally occurring amino acid
  misc_feature (17790)..(17791): Xaa can be any naturally occurring amino acid
  misc_feature (17793)..(17793): Xaa can be any naturally occurring amino acid
  misc_feature (17803)..(17803): Xaa can be any naturally occurring amino acid
  misc_feature (17813)..(17814): Xaa can be any naturally occurring amino acid
  misc_feature (17816)..(17816): Xaa can be any naturally occurring amino acid
  misc_feature (17828)..(17829): Xaa can be any naturally occurring amino acid
  misc_feature (17838)..(17838): Xaa can be any naturally occurring amino acid
  misc_feature (17840)..(17841): Xaa can be any naturally occurring amino acid
  misc_feature (17843)..(17845): Xaa can be any naturally occurring amino acid
  misc_feature (17849)..(17849): Xaa can be any naturally occurring amino acid
  misc_feature (17853)..(17853): Xaa can be any naturally occurring amino acid
  misc_feature (17855)..(17855): Xaa can be any naturally occurring amino acid
  misc_feature (17861)..(17865): Xaa can be any naturally occurring amino acid
  misc_feature (17867)..(17868): Xaa can be any naturally occurring amino acid
  misc_feature (17879)..(17879): Xaa can be any naturally occurring amino acid
  misc_feature (17886)..(17886): Xaa can be any naturally occurring amino acid
  misc_feature (17888)..(17888): Xaa can be any naturally occurring amino acid
  misc_feature (17892)..(17892): Xaa can be any naturally occurring amino acid
  misc_feature (17896)..(17896): Xaa can be any naturally occurring amino acid
  misc_feature (17905)..(17905): Xaa can be any naturally occurring amino acid
  misc_feature (17908)..(17908): Xaa can be any naturally occurring amino acid
  misc_feature (17911)..(17911): Xaa can be any naturally occurring amino acid
  misc_feature (17917)..(17917): Xaa can be any naturally occurring amino acid
  misc_feature (17922)..(17922): Xaa can be any naturally occurring amino acid
  misc_feature (17924)..(17926): Xaa can be any naturally occurring amino acid
  misc_feature (17929)..(17929): Xaa can be any naturally occurring amino acid
  misc_feature (17933)..(17934): Xaa can be any naturally occurring amino acid
  misc_feature (17936)..(17936): Xaa can be any naturally occurring amino acid
  misc_feature (17943)..(17943): Xaa can be any naturally occurring amino acid
  misc_feature (17946)..(17947): Xaa can be any naturally occurring amino acid
  misc_feature (17949)..(17949): Xaa can be any naturally occurring amino acid
  misc_feature (17959)..(17959): Xaa can be any naturally occurring amino acid
  misc_feature (18089)..(18090): Xaa can be any naturally occurring amino acid
  misc_feature (18092)..(18092): Xaa can be any naturally occurring amino acid
  misc_feature (18099)..(18099): Xaa can be any naturally occurring amino acid
  misc_feature (18102)..(18103): Xaa can be any naturally occurring amino acid
  misc_feature (18105)..(18105): Xaa can be any naturally occurring amino acid
  misc_feature (18115)..(18115): Xaa can be any naturally occurring amino acid
  misc_feature (18125)..(18126): Xaa can be any naturally occurring amino acid
  misc_feature (18128)..(18128): Xaa can be any naturally occurring amino acid
  misc_feature (18140)..(18141): Xaa can be any naturally occurring amino acid
  misc_feature (18150)..(18150): Xaa can be any naturally occurring amino acid
  misc_feature (18152)..(18153): Xaa can be any naturally occurring amino acid
  misc_feature (18155)..(18157): Xaa can be any naturally occurring amino acid
  misc_feature (18161)..(18161): Xaa can be any naturally occurring amino acid
  misc_feature (18165)..(18165): Xaa can be any naturally occurring amino acid
  misc_feature (18167)..(18167): Xaa can be any naturally occurring amino acid
  misc_feature (18173)..(18177): Xaa can be any naturally occurring amino acid
  misc_feature (18179)..(18180): Xaa can be any naturally occurring amino acid
  misc_feature (18191)..(18191): Xaa can be any naturally occurring amino acid
  misc_feature (18198)..(18198): Xaa can be any naturally occurring amino acid
  misc_feature (18200)..(18200): Xaa can be any naturally occurring amino acid
  misc_feature (18204)..(18204): Xaa can be any naturally occurring amino acid
  misc_feature (18208)..(18208): Xaa can be any naturally occurring amino acid
  misc_feature (18217)..(18217): Xaa can be any naturally occurring amino acid
  misc_feature (18220)..(18220): Xaa can be any naturally occurring amino acid
  misc_feature (18223)..(18223): Xaa can be any naturally occurring amino acid
  misc_feature (18229)..(18229): Xaa can be any naturally occurring amino acid
  misc_feature (18234)..(18234): Xaa can be any naturally occurring amino acid
  misc_feature (18236)..(18238): Xaa can be any naturally occurring amino acid
  misc_feature (18241)..(18241): Xaa can be any naturally occurring amino acid
  misc_feature (18245)..(18246): Xaa can be any naturally occurring amino acid
  misc_feature (18248)..(18248): Xaa can be any naturally occurring amino acid
  misc_feature (18255)..(18255): Xaa can be any naturally occurring amino acid
  misc_feature (18258)..(18259): Xaa can be any naturally occurring amino acid
  misc_feature (18261)..(18261): Xaa can be any naturally occurring amino acid
  misc_feature (18271)..(18271): Xaa can be any naturally occurring amino acid
  misc_feature (18401)..(18402): Xaa can be any naturally occurring amino acid
  misc_feature (18404)..(18404): Xaa can be any naturally occurring amino acid
  misc_feature (18411)..(18411): Xaa can be any naturally occurring amino acid
  misc_feature (18414)..(18415): Xaa can be any naturally occurring amino acid
  misc_feature (18417)..(18417): Xaa can be any naturally occurring amino acid
  misc_feature (18427)..(18427): Xaa can be any naturally occurring amino acid
  misc_feature (18437)..(18438): Xaa can be any naturally occurring amino acid
  misc_feature (18440)..(18440): Xaa can be any naturally occurring amino acid
  misc_feature (18452)..(18453): Xaa can be any naturally occurring amino acid
  misc_feature (18462)..(18462): Xaa can be any naturally occurring amino acid
  misc_feature (18464)..(18465): Xaa can be any naturally occurring amino acid
  misc_feature (18467)..(18469): Xaa can be any naturally occurring amino acid
  misc_feature (18473)..(18473): Xaa can be any naturally occurring amino acid
  misc_feature (18477)..(18477): Xaa can be any naturally occurring amino acid
  misc_feature (18479)..(18479): Xaa can be any naturally occurring amino acid
  misc_feature (18485)..(18489): Xaa can be any naturally occurring amino acid
  misc_feature (18491)..(18492): Xaa can be any naturally occurring amino acid
  misc_feature (18503)..(18503): Xaa can be any naturally occurring amino acid
  misc_feature (18510)..(18510): Xaa can be any naturally occurring amino acid
  misc_feature (18512)..(18512): Xaa can be any naturally occurring amino acid
  misc_feature (18516)..(18516): Xaa can be any naturally occurring amino acid
  misc_feature (18520)..(18520): Xaa can be any naturally occurring amino acid
  misc_feature (18529)..(18529): Xaa can be any naturally occurring amino acid
  misc_feature (18532)..(18532): Xaa can be any naturally occurring amino acid
  misc_feature (18535)..(18535): Xaa can be any naturally occurring amino acid
  misc_feature (18541)..(18541): Xaa can be any naturally occurring amino acid
  misc_feature (18546)..(18546): Xaa can be any naturally occurring amino acid
  misc_feature (18548)..(18550): Xaa can be any naturally occurring amino acid
  misc_feature (18553)..(18553): Xaa can be any naturally occurring amino acid
  misc_feature (18557)..(18558): Xaa can be any naturally occurring amino acid
  misc_feature (18560)..(18560): Xaa can be any naturally occurring amino acid
  misc_feature (18567)..(18567): Xaa can be any naturally occurring amino acid
  misc_feature (18570)..(18571): Xaa can be any naturally occurring amino acid
  misc_feature (18573)..(18573): Xaa can be any naturally occurring amino acid
  misc_feature (18583)..(18583): Xaa can be any naturally occurring amino acid
  misc_feature (18713)..(18714): Xaa can be any naturally occurring amino acid
  misc_feature (18716)..(18716): Xaa can be any naturally occurring amino acid
  misc_feature (18723)..(18723): Xaa can be any naturally occurring amino acid
  misc_feature (18726)..(18727): Xaa can be any naturally occurring amino acid
  misc_feature (18729)..(18729): Xaa can be any naturally occurring amino acid
  misc_feature (18739)..(18739): Xaa can be any naturally occurring amino acid
  misc_feature (18749)..(18750): Xaa can be any naturally occurring amino acid
  misc_feature (18752)..(18752): Xaa can be any naturally occurring amino acid
  misc_feature (18764)..(18765): Xaa can be any naturally occurring amino acid
  misc_feature (18774)..(18774): Xaa can be any naturally occurring amino acid
  misc_feature (18776)..(18777): Xaa can be any naturally occurring amino acid
  misc_feature (18779)..(18781): Xaa can be any naturally occurring amino acid
  misc_feature (18785)..(18785): Xaa can be any naturally occurring amino acid
  misc_feature (18789)..(18789): Xaa can be any naturally occurring amino acid
  misc_feature (18791)..(18791): Xaa can be any naturally occurring amino acid
  misc_feature (18797)..(18801): Xaa can be any naturally occurring amino acid
  misc_feature (18803)..(18804): Xaa can be any naturally occurring amino acid
  misc_feature (18815)..(18815): Xaa can be any naturally occurring amino acid
  misc_feature (18822)..(18822): Xaa can be any naturally occurring amino acid
  misc_feature (18824)..(18824): Xaa can be any naturally occurring amino acid
  misc_feature (18828)..(18828): Xaa can be any naturally occurring amino acid
  misc_feature (18832)..(18832): Xaa can be any naturally occurring amino acid
  misc_feature (18841)..(18841): Xaa can be any naturally occurring amino acid
  misc_feature (18844)..(18844): Xaa can be any naturally occurring amino acid
  misc_feature (18847)..(18847): Xaa can be any naturally occurring amino acid
  misc_feature (18853)..(18853): Xaa can be any naturally occurring amino acid
  misc_feature (18858)..(18858): Xaa can be any naturally occurring amino acid
  misc_feature (18860)..(18862): Xaa can be any naturally occurring amino acid
  misc_feature (18865)..(18865): Xaa can be any naturally occurring amino acid
  misc_feature (18869)..(18870): Xaa can be any naturally occurring amino acid
  misc_feature (18872)..(18872): Xaa can be any naturally occurring amino acid
  misc_feature (18879)..(18879): Xaa can be any naturally occurring amino acid
  misc_feature (18882)..(18883): Xaa can be any naturally occurring amino acid
  misc_feature (18885)..(18885): Xaa can be any naturally occurring amino acid
  misc_feature (18895)..(18895): Xaa can be any naturally occurring amino acid
  misc_feature (19091)..(19093): Xaa can be any naturally occurring amino acid
  misc_feature (19097)..(19097): Xaa can be any naturally occurring amino acid
  misc_feature (19101)..(19101): Xaa can be any naturally occurring amino acid
  misc_feature (19103)..(19103): Xaa can be any naturally occurring amino acid
  misc_feature (19109)..(19113): Xaa can be any naturally occurring amino acid
  misc_feature (19115)..(19116): Xaa can be any naturally occurring amino acid
  misc_feature (19127)..(19127): Xaa can be any naturally occurring amino acid
  misc_feature (19134)..(19134): Xaa can be any naturally occurring amino acid
  misc_feature (19136)..(19136): Xaa can be any naturally occurring amino acid
  misc_feature (19140)..(19140): Xaa can be any naturally occurring amino acid
  misc_feature (19144)..(19144): Xaa can be any naturally occurring amino acid
  misc_feature (19153)..(19153): Xaa can be any naturally occurring amino acid
  misc_feature (19156)..(19156): Xaa can be any naturally occurring amino acid
  misc_feature (19159)..(19159): Xaa can be any naturally occurring amino acid
  misc_feature (19165)..(19165): Xaa can be any naturally occurring amino acid
  misc_feature (19170)..(19170): Xaa can be any naturally occurring amino acid
  misc_feature (19172)..(19174): Xaa can be any naturally occurring amino acid
  misc_feature (19177)..(19177): Xaa can be any naturally occurring amino acid
  misc_feature (19181)..(19182): Xaa can be any naturally occurring amino acid
  misc_feature (19184)..(19184): Xaa can be any naturally occurring amino acid
  misc_feature (19191)..(19191): Xaa can be any naturally occurring amino acid
  misc_feature (19194)..(19195): Xaa can be any naturally occurring amino acid
  misc_feature (19197)..(19197): Xaa can be any naturally occurring amino acid
  misc_feature (19207)..(19207): Xaa can be any naturally occurring amino acid
  misc_feature (19337)..(19338): Xaa can be any naturally occurring amino acid
  misc_feature (19340)..(19340): Xaa can be any naturally occurring amino acid
  misc_feature (19347)..(19347): Xaa can be any naturally occurring amino acid
  misc_feature (19350)..(19351): Xaa can be any naturally occurring amino acid
  misc_feature (19353)..(19353): Xaa can be any naturally occurring amino acid
  misc_feature (19363)..(19363): Xaa can be any naturally occurring amino acid
  misc_feature (19373)..(19374): Xaa can be any naturally occurring amino acid
  misc_feature (19376)..(19376): Xaa can be any naturally occurring amino acid
  misc_feature (19388)..(19389): Xaa can be any naturally occurring amino acid
  misc_feature (19398)..(19398): Xaa can be any naturally occurring amino acid
  misc_feature (19400)..(19401): Xaa can be any naturally occurring amino acid
  misc_feature (19403)..(19405): Xaa can be any naturally occurring amino acid
  misc_feature (19409)..(19409): Xaa can be any naturally occurring amino acid
  misc_feature (19413)..(19413): Xaa can be any naturally occurring amino acid
  misc_feature (19415)..(19415): Xaa can be any naturally occurring amino acid
  misc_feature (19421)..(19425): Xaa can be any naturally occurring amino acid
  misc_feature (19427)..(19428): Xaa can be any naturally occurring amino acid
  misc_feature (19439)..(19439): Xaa can be any naturally occurring amino acid
  misc_feature (19446)..(19446): Xaa can be any naturally occurring amino acid
  misc_feature (19448)..(19448): Xaa can be any naturally occurring amino acid
  misc_feature (19452)..(19452): Xaa can be any naturally occurring amino acid
  misc_feature (19456)..(19456): Xaa can be any naturally occurring amino acid
  misc_feature (19465)..(19465): Xaa can be any naturally occurring amino acid
  misc_feature (19468)..(19468): Xaa can be any naturally occurring amino acid
  misc_feature (19471)..(19471): Xaa can be any naturally occurring amino acid
  misc_feature (19477)..(19477): Xaa can be any naturally occurring amino acid
  misc_feature (19482)..(19482): Xaa can be any naturally occurring amino acid
  misc_feature (19484)..(19486): Xaa can be any naturally occurring amino acid
  misc_feature (19489)..(19489): Xaa can be any naturally occurring amino acid
  misc_feature (19493)..(19494): Xaa can be any naturally occurring amino acid
  misc_feature (19496)..(19496): Xaa can be any naturally occurring amino acid
  misc_feature (19503)..(19503): Xaa can be any naturally occurring amino acid
  misc_feature (19506)..(19507): Xaa can be any naturally occurring amino acid
  misc_feature (19509)..(19509): Xaa can be any naturally occurring amino acid
  misc_feature (19519)..(19519): Xaa can be any naturally occurring amino acid
  misc_feature (19649)..(19650): Xaa can be any naturally occurring amino acid
  misc_feature (19652)..(19652): Xaa can be any naturally occurring amino acid
  misc_feature (19659)..(19659): Xaa can be any naturally occurring amino acid
  misc_feature (19662)..(19663): Xaa can be any naturally occurring amino acid
  misc_feature (19665)..(19665): Xaa can be any naturally occurring amino acid
  misc_feature (19675)..(19675): Xaa can be any naturally occurring amino acid
  misc_feature (19685)..(19686): Xaa can be any naturally occurring amino acid
  misc_feature (19688)..(19688): Xaa can be any naturally occurring amino acid
  misc_feature (19700)..(19701): Xaa can be any naturally occurring amino acid
  misc_feature (19710)..(19710): Xaa can be any naturally occurring amino acid
  misc_feature (19712)..(19713): Xaa can be any naturally occurring amino acid
  misc_feature (19715)..(19717): Xaa can be any naturally occurring amino acid
  misc_feature (19721)..(19721): Xaa can be any naturally occurring amino acid
  misc_feature (19725)..(19725): Xaa can be any naturally occurring amino acid
  misc_feature (19727)..(19727): Xaa can be any naturally occurring amino acid
  misc_feature (19733)..(19737): Xaa can be any naturally occurring amino acid
  misc_feature (19739)..(19740): Xaa can be any naturally occurring amino acid
  misc_feature (19751)..(19751): Xaa can be any naturally occurring amino acid
  misc_feature (19758)..(19758): Xaa can be any naturally occurring amino acid
  misc_feature (19760)..(19760): Xaa can be any naturally occurring amino acid
  misc_feature (19764)..(19764): Xaa can be any naturally occurring amino acid
  misc_feature (19768)..(19768): Xaa can be any naturally occurring amino acid
  misc_feature (19777)..(19777): Xaa can be any naturally occurring amino acid
  misc_feature (19780)..(19780): Xaa can be any naturally occurring amino acid
  misc_feature (19783)..(19783): Xaa can be any naturally occurring amino acid
  misc_feature (19789)..(19789): Xaa can be any naturally occurring amino acid
  misc_feature (19794)..(19794): Xaa can be any naturally occurring amino acid
  misc_feature (19796)..(19798): Xaa can be any naturally occurring amino acid
  misc_feature (19801)..(19801): Xaa can be any naturally occurring amino acid
  misc_feature (19805)..(19806): Xaa can be any naturally occurring amino acid
  misc_feature (19808)..(19808): Xaa can be any naturally occurring amino acid
  misc_feature (19815)..(19815): Xaa can be any naturally occurring amino acid
  misc_feature (19818)..(19819): Xaa can be any naturally occurring amino acid
  misc_feature (19821)..(19821): Xaa can be any naturally occurring amino acid
  misc_feature (19831)..(19831): Xaa can be any naturally occurring amino acid
  misc_feature (19960)..(19961): Xaa can be any naturally occurring amino acid
  misc_feature (19963)..(19963): Xaa can be any naturally occurring amino acid
  misc_feature (19970)..(19970): Xaa can be any naturally occurring amino acid
  misc_feature (19973)..(19974): Xaa can be any naturally occurring amino acid
  misc_feature (19976)..(19976): Xaa can be any naturally occurring amino acid
  misc_feature (19986)..(19986): Xaa can be any naturally occurring amino acid
  misc_feature (19996)..(19997): Xaa can be any naturally occurring amino acid
  misc_feature (19999)..(19999): Xaa can be any naturally occurring amino acid
  misc_feature (20011)..(20012): Xaa can be any naturally occurring amino acid
  misc_feature (20021)..(20021): Xaa can be any naturally occurring amino acid
  misc_feature (20023)..(20024): Xaa can be any naturally occurring amino acid
  misc_feature (20026)..(20028): Xaa can be any naturally occurring amino acid
  misc_feature (20032)..(20032): Xaa can be any naturally occurring amino acid
  misc_feature (20036)..(20036): Xaa can be any naturally occurring amino acid
  misc_feature (20038)..(20038): Xaa can be any naturally occurring amino acid
  misc_feature (20044)..(20048): Xaa can be any naturally occurring amino acid
  misc_feature (20050)..(20051): Xaa can be any naturally occurring amino acid
  misc_feature (20062)..(20062): Xaa can be any naturally occurring amino acid
  misc_feature (20069)..(20069): Xaa can be any naturally occurring amino acid
  misc_feature (20071)..(20071): Xaa can be any naturally occurring amino acid
  misc_feature (20075)..(20075): Xaa can be any naturally occurring amino acid
  misc_feature (20079)..(20079): Xaa can be any naturally occurring amino acid
  misc_feature (20088)..(20088): Xaa can be any naturally occurring amino acid
  misc_feature (20091)..(20091): Xaa can be any naturally occurring amino acid
  misc_feature (20094)..(20094): Xaa can be any naturally occurring amino acid
  misc_feature (20100)..(20100): Xaa can be any naturally occurring amino acid
  misc_feature (20105)..(20105): Xaa can be any naturally occurring amino acid
  misc_feature (20107)..(20109): Xaa can be any naturally occurring amino acid
  misc_feature (20112)..(20112): Xaa can be any naturally occurring amino acid
  misc_feature (20116)..(20117): Xaa can be any naturally occurring amino acid
  misc_feature (20119)..(20119): Xaa can be any naturally occurring amino acid
  misc_feature (20126)..(20126): Xaa can be any naturally occurring amino acid
  misc_feature (20129)..(20130): Xaa can be any naturally occurring amino acid
  misc_feature (20132)..(20132): Xaa can be any naturally occurring amino acid
  misc_feature (20142)..(20142): Xaa can be any naturally occurring amino acid
  misc_feature (20272)..(20273): Xaa can be any naturally occurring amino acid
  misc_feature (20275)..(20275): Xaa can be any naturally occurring amino acid
  misc_feature (20282)..(20282): Xaa can be any naturally occurring amino acid
  misc_feature (20285)..(20286): Xaa can be any naturally occurring amino acid
  misc_feature (20288)..(20288): Xaa can be any naturally occurring amino acid
  misc_feature (20298)..(20298): Xaa can be any naturally occurring amino acid
  misc_feature (20308)..(20309): Xaa can be any naturally occurring amino acid
  misc_feature (20311)..(20311): Xaa can be any naturally occurring amino acid
  misc_feature (20323)..(20324): Xaa can be any naturally occurring amino acid
  misc_feature (20333)..(20333): Xaa can be any naturally occurring amino acid
  misc_feature (20335)..(20336): Xaa can be any naturally occurring amino acid
  misc_feature (20806)..(20808): Xaa can be any naturally occurring amino acid
  misc_feature (20812)..(20812): Xaa can be any naturally occurring amino acid
  misc_feature (20816)..(20816): Xaa can be any naturally occurring amino acid
  misc_feature (20818)..(20818): Xaa can be any naturally occurring amino acid
  misc_feature (20824)..(20828): Xaa can be any naturally occurring amino acid
  misc_feature (20830)..(20831): Xaa can be any naturally occurring amino acid
  ref <308>: DATABASE ACCESSION NUMBER: GenBank / AAL65133
  ref <309>: DATABASE ENTRY DATE: 2002-10-29
  ref <313>: RELEVANT RESIDUES: (1)..(22152)
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 2 - DNA, 66765 bp, Homo sapiens
  misc_feature (38757)..(38757): n is a, c, g, or t
  misc_feature (38775)..(38775): n is a, c, g, or t
  misc_feature (38778)..(38778): n is a, c, g, or t
  misc_feature (38835)..(38835): n is a, c, g, or t
  misc_feature (38838)..(38838): n is a, c, g, or t
  misc_feature (38859)..(38859): n is a, c, g, or t
  misc_feature (38868)..(38868): n is a, c, g, or t
  misc_feature (38883)..(38883): n is a, c, g, or t
  misc_feature (38892)..(38892): n is a, c, g, or t
  misc_feature (41833)..(41833): n is a, c, g, or t
  misc_feature (41838)..(41838): n is a, c, g, or t
  misc_feature (41843)..(41843): n is a, c, g, or t
  misc_feature (41847)..(41847): n is a, c, g, or t
  misc_feature (41859)..(41859): n is a, c, g, or t
  misc_feature (41865)..(41865): n is a, c, g, or t
  misc_feature (41874)..(41876): n is a, c, g, or t
  misc_feature (41881)..(41883): n is a, c, g, or t
  misc_feature (41892)..(41892): n is a, c, g, or t
  misc_feature (41911)..(41911): n is a, c, g, or t
  misc_feature (41942)..(41942): n is a, c, g, or t
  misc_feature (41945)..(41945): n is a, c, g, or t
  misc_feature (41951)..(41951): n is a, c, g, or t
  misc_feature (41986)..(41986): n is a, c, g, or t
  misc_feature (41989)..(41989): n is a, c, g, or t
  misc_feature (41991)..(41991): n is a, c, g, or t
  misc_feature (41994)..(41994): n is a, c, g, or t
  misc_feature (42000)..(42000): n is a, c, g, or t
  misc_feature (42016)..(42016): n is a, c, g, or t
  misc_feature (42022)..(42022): n is a, c, g, or t
  misc_feature (42026)..(42026): n is a, c, g, or t
  misc_feature (43909)..(43909): n is a, c, g, or t
  misc_feature (43911)..(43912): n is a, c, g, or t
  misc_feature (43916)..(43916): n is a, c, g, or t
  misc_feature (43927)..(43927): n is a, c, g, or t
  misc_feature (43929)..(43929): n is a, c, g, or t
  misc_feature (43932)..(43932): n is a, c, g, or t
  misc_feature (43939)..(43939): n is a, c, g, or t
  misc_feature (43947)..(43947): n is a, c, g, or t
  misc_feature (43965)..(43965): n is a, c, g, or t
  misc_feature (43968)..(43968): n is a, c, g, or t
  misc_feature (43970)..(43970): n is a, c, g, or t
  misc_feature (43974)..(43975): n is a, c, g, or t
  misc_feature (43982)..(43983): n is a, c, g, or t
  misc_feature (43985)..(43985): n is a, c, g, or t
  misc_feature (43989)..(43989): n is a, c, g, or t
  misc_feature (43992)..(43992): n is a, c, g, or t
  misc_feature (44013)..(44013): n is a, c, g, or t
  misc_feature (44377)..(44377): n is a, c, g, or t
  misc_feature (44379)..(44380): n is a, c, g, or t
  misc_feature (44384)..(44384): n is a, c, g, or t
  misc_feature (44395)..(44395): n is a, c, g, or t
  misc_feature (44397)..(44397): n is a, c, g, or t
  misc_feature (44400)..(44400): n is a, c, g, or t
  misc_feature (44407)..(44407): n is a, c, g, or t
  misc_feature (44415)..(44415): n is a, c, g, or t
  misc_feature (44433)..(44433): n is a, c, g, or t
  misc_feature (44436)..(44436): n is a, c, g, or t
  misc_feature (44438)..(44438): n is a, c, g, or t
  misc_feature (44442)..(44443): n is a, c, g, or t
  misc_feature (44450)..(44451): n is a, c, g, or t
  misc_feature (44453)..(44453): n is a, c, g, or t
  misc_feature (44457)..(44457): n is a, c, g, or t
  misc_feature (44460)..(44460): n is a, c, g, or t
  misc_feature (44481)..(44481): n is a, c, g, or t
  misc_feature (47185)..(47185): n is a, c, g, or t
  misc_feature (47187)..(47188): n is a, c, g, or t
  misc_feature (47192)..(47192): n is a, c, g, or t
  misc_feature (47203)..(47203): n is a, c, g, or t
  misc_feature (47205)..(47205): n is a, c, g, or t
  misc_feature (47208)..(47208): n is a, c, g, or t
  misc_feature (47215)..(47215): n is a, c, g, or t
  misc_feature (47223)..(47223): n is a, c, g, or t
  misc_feature (47241)..(47241): n is a, c, g, or t
  misc_feature (47244)..(47244): n is a, c, g, or t
  misc_feature (47246)..(47246): n is a, c, g, or t
  misc_feature (47250)..(47251): n is a, c, g, or t
  misc_feature (47258)..(47259): n is a, c, g, or t
  misc_feature (47261)..(47261): n is a, c, g, or t
  misc_feature (47265)..(47265): n is a, c, g, or t
  misc_feature (47268)..(47268): n is a, c, g, or t
  misc_feature (47289)..(47289): n is a, c, g, or t
  misc_feature (48118)..(48118): n is a, c, g, or t
  misc_feature (48120)..(48121): n is a, c, g, or t
  misc_feature (48125)..(48125): n is a, c, g, or t
  misc_feature (48136)..(48136): n is a, c, g, or t
  misc_feature (48138)..(48138): n is a, c, g, or t
  misc_feature (48141)..(48141): n is a, c, g, or t
  misc_feature (48148)..(48148): n is a, c, g, or t
  misc_feature (48156)..(48156): n is a, c, g, or t
  misc_feature (48174)..(48174): n is a, c, g, or t
  misc_feature (48177)..(48177): n is a, c, g, or t
  misc_feature (48179)..(48179): n is a, c, g, or t
  misc_feature (48183)..(48184): n is a, c, g, or t
  misc_feature (48191)..(48192): n is a, c, g, or t
  misc_feature (48194)..(48194): n is a, c, g, or t
  misc_feature (48198)..(48198): n is a, c, g, or t
  misc_feature (48201)..(48201): n is a, c, g, or t
  misc_feature (48222)..(48222): n is a, c, g, or t
  misc_feature (48226)..(48226): n is a, c, g, or t
  misc_feature (48228)..(48228): n is a, c, g, or t
  misc_feature (48231)..(48231): n is a, c, g, or t
  misc_feature (48246)..(48249): n is a, c, g, or t
  misc_feature (48253)..(48253): n is a, c, g, or t
  misc_feature (48255)..(48255): n is a, c, g, or t
  misc_feature (48265)..(48265): n is a, c, g, or t
  misc_feature (48273)..(48273): n is a, c, g, or t
  misc_feature (48278)..(48278): n is a, c, g, or t
  misc_feature (48305)..(48306): n is a, c, g, or t
  misc_feature (48313)..(48313): n is a, c, g, or t
  misc_feature (48315)..(48315): n is a, c, g, or t
  misc_feature (48322)..(48322): n is a, c, g, or t
  misc_feature (48327)..(48327): n is a, c, g, or t
  misc_feature (48340)..(48340): n is a, c, g, or t
  misc_feature (48356)..(48356): n is a, c, g, or t
  misc_feature (48362)..(48363): n is a, c, g, or t
  misc_feature (48365)..(48365): n is a, c, g, or t
  misc_feature (48367)..(48367): n is a, c, g, or t
  misc_feature (48369)..(48369): n is a, c, g, or t
  misc_feature (48378)..(48378): n is a, c, g, or t
  misc_feature (48388)..(48388): n is a, c, g, or t
  misc_feature (48393)..(48393): n is a, c, g, or t
  misc_feature (48398)..(48398): n is a, c, g, or t
  misc_feature (48402)..(48402): n is a, c, g, or t
  misc_feature (48414)..(48414): n is a, c, g, or t
  misc_feature (48420)..(48420): n is a, c, g, or t
  misc_feature (48429)..(48431): n is a, c, g, or t
  misc_feature (48436)..(48438): n is a, c, g, or t
  misc_feature (48447)..(48447): n is a, c, g, or t
  misc_feature (48466)..(48466): n is a, c, g, or t
  misc_feature (49006)..(49006): n is a, c, g, or t
  misc_feature (49009)..(49009): n is a, c, g, or t
  misc_feature (49011)..(49011): n is a, c, g, or t
  misc_feature (49014)..(49014): n is a, c, g, or t
  misc_feature (49020)..(49020): n is a, c, g, or t
  misc_feature (49036)..(49036): n is a, c, g, or t
  misc_feature (49042)..(49042): n is a, c, g, or t
  misc_feature (49046)..(49046): n is a, c, g, or t
  misc_feature (49321)..(49321): n is a, c, g, or t
  misc_feature (49326)..(49326): n is a, c, g, or t
  misc_feature (49331)..(49331): n is a, c, g, or t
  misc_feature (49335)..(49335): n is a, c, g, or t
  misc_feature (49347)..(49347): n is a, c, g, or t
  misc_feature (49353)..(49353): n is a, c, g, or t
  misc_feature (49362)..(49364): n is a, c, g, or t
  misc_feature (49369)..(49371): n is a, c, g, or t
  misc_feature (49380)..(49380): n is a, c, g, or t
  misc_feature (49399)..(49399): n is a, c, g, or t
  misc_feature (49430)..(49430): n is a, c, g, or t
  misc_feature (49433)..(49433): n is a, c, g, or t
  misc_feature (49439)..(49439): n is a, c, g, or t
  misc_feature (49474)..(49474): n is a, c, g, or t
  misc_feature (49477)..(49477): n is a, c, g, or t
  misc_feature (49479)..(49479): n is a, c, g, or t
  misc_feature (49482)..(49482): n is a, c, g, or t
  misc_feature (49488)..(49488): n is a, c, g, or t
  misc_feature (49504)..(49504): n is a, c, g, or t
  misc_feature (49510)..(49510): n is a, c, g, or t
  misc_feature (49514)..(49514): n is a, c, g, or t
  misc_feature (49519)..(49519): n is a, c, g, or t
  misc_feature (49521)..(49522): n is a, c, g, or t
  misc_feature (49526)..(49526): n is a, c, g, or t
  misc_feature (49537)..(49537): n is a, c, g, or t
  misc_feature (49539)..(49539): n is a, c, g, or t
  misc_feature (49542)..(49542): n is a, c, g, or t
  misc_feature (49549)..(49549): n is a, c, g, or t
  misc_feature (49557)..(49557): n is a, c, g, or t
  misc_feature (49575)..(49575): n is a, c, g, or t
  misc_feature (49578)..(49578): n is a, c, g, or t
  misc_feature (49580)..(49580): n is a, c, g, or t
  misc_feature (49584)..(49585): n is a, c, g, or t
  misc_feature (49592)..(49593): n is a, c, g, or t
  misc_feature (49595)..(49595): n is a, c, g, or t
  misc_feature (49599)..(49599): n is a, c, g, or t
  misc_feature (49602)..(49602): n is a, c, g, or t
  misc_feature (49623)..(49623): n is a, c, g, or t
  misc_feature (50725)..(50725): n is a, c, g, or t
  misc_feature (50730)..(50730): n is a, c, g, or t
  misc_feature (50735)..(50735): n is a, c, g, or t
  misc_feature (50739)..(50739): n is a, c, g, or t
  misc_feature (50751)..(50751): n is a, c, g, or t
  misc_feature (50757)..(50757): n is a, c, g, or t
  misc_feature (50766)..(50768): n is a, c, g, or t
  misc_feature (50773)..(50775): n is a, c, g, or t
  misc_feature (50784)..(50784): n is a, c, g, or t
  misc_feature (50803)..(50803): n is a, c, g, or t
  misc_feature (50834)..(50834): n is a, c, g, or t
  misc_feature (50837)..(50837): n is a, c, g, or t
  misc_feature (50843)..(50843): n is a, c, g, or t
  misc_feature (50878)..(50878): n is a, c, g, or t
  misc_feature (50881)..(50881): n is a, c, g, or t
  misc_feature (50883)..(50883): n is a, c, g, or t
  misc_feature (50886)..(50886): n is a, c, g, or t
  misc_feature (50892)..(50892): n is a, c, g, or t
  misc_feature (50908)..(50908): n is a, c, g, or t
  misc_feature (50914)..(50914): n is a, c, g, or t
  misc_feature (50918)..(50918): n is a, c, g, or t
  misc_feature (50923)..(50923): n is a, c, g, or t
  misc_feature (50925)..(50926): n is a, c, g, or t
  misc_feature (50930)..(50930): n is a, c, g, or t
  misc_feature (50941)..(50941): n is a, c, g, or t
  misc_feature (50943)..(50943): n is a, c, g, or t
  misc_feature (50946)..(50946): n is a, c, g, or t
  misc_feature (50953)..(50953): n is a, c, g, or t
  misc_feature (50961)..(50961): n is a, c, g, or t
  misc_feature (50979)..(50979): n is a, c, g, or t
  misc_feature (50982)..(50982): n is a, c, g, or t
  misc_feature (50984)..(50984): n is a, c, g, or t
  misc_feature (50988)..(50989): n is a, c, g, or t
  misc_feature (50996)..(50997): n is a, c, g, or t
  misc_feature (50999)..(50999): n is a, c, g, or t
  misc_feature (51003)..(51003): n is a, c, g, or t
  misc_feature (51006)..(51006): n is a, c, g, or t
  misc_feature (51027)..(51027): n is a, c, g, or t
  misc_feature (52597)..(52597): n is a, c, g, or t
  misc_feature (52602)..(52602): n is a, c, g, or t
  misc_feature (52607)..(52607): n is a, c, g, or t
  misc_feature (52611)..(52611): n is a, c, g, or t
  misc_feature (52623)..(52623): n is a, c, g, or t
  misc_feature (52629)..(52629): n is a, c, g, or t
  misc_feature (52638)..(52640): n is a, c, g, or t
  misc_feature (52645)..(52647): n is a, c, g, or t
  misc_feature (52656)..(52656): n is a, c, g, or t
  misc_feature (52675)..(52675): n is a, c, g, or t
  misc_feature (52706)..(52706): n is a, c, g, or t
  misc_feature (52709)..(52709): n is a, c, g, or t
  misc_feature (52715)..(52715): n is a, c, g, or t
  misc_feature (52750)..(52750): n is a, c, g, or t
  misc_feature (52753)..(52753): n is a, c, g, or t
  misc_feature (52755)..(52755): n is a, c, g, or t
  misc_feature (52758)..(52758): n is a, c, g, or t
  misc_feature (52764)..(52764): n is a, c, g, or t
  misc_feature (52780)..(52780): n is a, c, g, or t
  misc_feature (52786)..(52786): n is a, c, g, or t
  misc_feature (52790)..(52790): n is a, c, g, or t
  misc_feature (52795)..(52795): n is a, c, g, or t
  misc_feature (52797)..(52798): n is a, c, g, or t
  misc_feature (52802)..(52802): n is a, c, g, or t
  misc_feature (52813)..(52813): n is a, c, g, or t
  misc_feature (52815)..(52815): n is a, c, g, or t
  misc_feature (52818)..(52818): n is a, c, g, or t
  misc_feature (52825)..(52825): n is a, c, g, or t
  misc_feature (52833)..(52833): n is a, c, g, or t
  misc_feature (52851)..(52851): n is a, c, g, or t
  misc_feature (52854)..(52854): n is a, c, g, or t
  misc_feature (52856)..(52856): n is a, c, g, or t
  misc_feature (52860)..(52861): n is a, c, g, or t
  misc_feature (52868)..(52869): n is a, c, g, or t
  misc_feature (52871)..(52871): n is a, c, g, or t
  misc_feature (52875)..(52875): n is a, c, g, or t
  misc_feature (52878)..(52878): n is a, c, g, or t
  misc_feature (52899)..(52899): n is a, c, g, or t
  misc_feature (52903)..(52903): n is a, c, g, or t
  misc_feature (52905)..(52905): n is a, c, g, or t
  misc_feature (52908)..(52908): n is a, c, g, or t
  misc_feature (52923)..(52926): n is a, c, g, or t
  misc_feature (52930)..(52930): n is a, c, g, or t
  misc_feature (52932)..(52932): n is a, c, g, or t
  misc_feature (52942)..(52942): n is a, c, g, or t
  misc_feature (52950)..(52950): n is a, c, g, or t
  misc_feature (52955)..(52955): n is a, c, g, or t
  misc_feature (52982)..(52983): n is a, c, g, or t
  misc_feature (52990)..(52990): n is a, c, g, or t
  misc_feature (52992)..(52992): n is a, c, g, or t
  misc_feature (52999)..(52999): n is a, c, g, or t
  misc_feature (53004)..(53004): n is a, c, g, or t
  misc_feature (53017)..(53017): n is a, c, g, or t
  misc_feature (53033)..(53033): n is a, c, g, or t
  misc_feature (53039)..(53040): n is a, c, g, or t
  misc_feature (53042)..(53042): n is a, c, g, or t
  misc_feature (53044)..(53044): n is a, c, g, or t
  misc_feature (53046)..(53046): n is a, c, g, or t
  misc_feature (53055)..(53055): n is a, c, g, or t
  misc_feature (53065)..(53065): n is a, c, g, or t
  misc_feature (53070)..(53070): n is a, c, g, or t
  misc_feature (53075)..(53075): n is a, c, g, or t
  misc_feature (53079)..(53079): n is a, c, g, or t
  misc_feature (53091)..(53091): n is a, c, g, or t
  misc_feature (53097)..(53097): n is a, c, g, or t
  misc_feature (53533)..(53533): n is a, c, g, or t
  misc_feature (53538)..(53538): n is a, c, g, or t
  misc_feature (53543)..(53543): n is a, c, g, or t
  misc_feature (53547)..(53547): n is a, c, g, or t
  misc_feature (53559)..(53559): n is a, c, g, or t
  misc_feature (53565)..(53565): n is a, c, g, or t
  misc_feature (53574)..(53576): n is a, c, g, or t
  misc_feature (53581)..(53583): n is a, c, g, or t
  misc_feature (53592)..(53592): n is a, c, g, or t
  misc_feature (53611)..(53611): n is a, c, g, or t
  misc_feature (53642)..(53642): n is a, c, g, or t
  misc_feature (53645)..(53645): n is a, c, g, or t
  misc_feature (53651)..(53651): n is a, c, g, or t
  misc_feature (53686)..(53686): n is a, c, g, or t
  misc_feature (53689)..(53689): n is a, c, g, or t
  misc_feature (53691)..(53691): n is a, c, g, or t
  misc_feature (53694)..(53694): n is a, c, g, or t
  misc_feature (53700)..(53700): n is a, c, g, or t
  misc_feature (53716)..(53716): n is a, c, g, or t
  misc_feature (53722)..(53722): n is a, c, g, or t
  misc_feature (53726)..(53726): n is a, c, g, or t
  misc_feature (53731)..(53731): n is a, c, g, or t
  misc_feature (53733)..(53734): n is a, c, g, or t
  misc_feature (53738)..(53738): n is a, c, g, or t
  misc_feature (53749)..(53749): n is a, c, g, or t
  misc_feature (53751)..(53751): n is a, c, g, or t
  misc_feature (53754)..(53754): n is a, c, g, or t
  misc_feature (53761)..(53761): n is a, c, g, or t
  misc_feature (53769)..(53769): n is a, c, g, or t
  misc_feature (53787)..(53787): n is a, c, g, or t
  misc_feature (53790)..(53790): n is a, c, g, or t
  misc_feature (53792)..(53792): n is a, c, g, or t
  misc_feature (53796)..(53797): n is a, c, g, or t
  misc_feature (53804)..(53805): n is a, c, g, or t
  misc_feature (53807)..(53807): n is a, c, g, or t
  misc_feature (53811)..(53811): n is a, c, g, or t
  misc_feature (53814)..(53814): n is a, c, g, or t
  misc_feature (53835)..(53835): n is a, c, g, or t
  misc_feature (53839)..(53839): n is a, c, g, or t
  misc_feature (53841)..(53841): n is a, c, g, or t
  misc_feature (53844)..(53844): n is a, c, g, or t
  misc_feature (53859)..(53862): n is a, c, g, or t
  misc_feature (53866)..(53866): n is a, c, g, or t
  misc_feature (53868)..(53868): n is a, c, g, or t
  misc_feature (53878)..(53878): n is a, c, g, or t
  misc_feature (53886)..(53886): n is a, c, g, or t
  misc_feature (53891)..(53891): n is a, c, g, or t
  misc_feature (53918)..(53919): n is a, c, g, or t
  misc_feature (53926)..(53926): n is a, c, g, or t
  misc_feature (53928)..(53928): n is a, c, g, or t
  misc_feature (53935)..(53935): n is a, c, g, or t
  misc_feature (53940)..(53940): n is a, c, g, or t
  misc_feature (53953)..(53953): n is a, c, g, or t
  misc_feature (53969)..(53969): n is a, c, g, or t
  misc_feature (53975)..(53976): n is a, c, g, or t
  misc_feature (53978)..(53978): n is a, c, g, or t
  misc_feature (53980)..(53980): n is a, c, g, or t
  misc_feature (53982)..(53982): n is a, c, g, or t
  misc_feature (53991)..(53991): n is a, c, g, or t
  misc_feature (54001)..(54001): n is a, c, g, or t
  misc_feature (54006)..(54006): n is a, c, g, or t
  misc_feature (54011)..(54011): n is a, c, g, or t
  misc_feature (54015)..(54015): n is a, c, g, or t
  misc_feature (54027)..(54027): n is a, c, g, or t
  misc_feature (54033)..(54033): n is a, c, g, or t
  misc_feature (54042)..(54044): n is a, c, g, or t
  misc_feature (54049)..(54051): n is a, c, g, or t
  misc_feature (54060)..(54060): n is a, c, g, or t
  misc_feature (54079)..(54079): n is a, c, g, or t
  misc_feature (54469)..(54469): n is a, c, g, or t
  misc_feature (54474)..(54474): n is a, c, g, or t
  misc_feature (54479)..(54479): n is a, c, g, or t
  misc_feature (54483)..(54483): n is a, c, g, or t
  misc_feature (54495)..(54495): n is a, c, g, or t
  misc_feature (54501)..(54501): n is a, c, g, or t
  misc_feature (54510)..(54512): n is a, c, g, or t
  misc_feature (54517)..(54519): n is a, c, g, or t
  misc_feature (54528)..(54528): n is a, c, g, or t
  misc_feature (54547)..(54547): n is a, c, g, or t
  misc_feature (54578)..(54578): n is a, c, g, or t
  misc_feature (54581)..(54581): n is a, c, g, or t
  misc_feature (54587)..(54587): n is a, c, g, or t
  misc_feature (54622)..(54622): n is a, c, g, or t
  misc_feature (54625)..(54625): n is a, c, g, or t
  misc_feature (54627)..(54627): n is a, c, g, or t
  misc_feature (54630)..(54630): n is a, c, g, or t
  misc_feature (54636)..(54636): n is a, c, g, or t
  misc_feature (54652)..(54652): n is a, c, g, or t
  misc_feature (54658)..(54658): n is a, c, g, or t
  misc_feature (54662)..(54662): n is a, c, g, or t
  misc_feature (54667)..(54667): n is a, c, g, or t
  misc_feature (54669)..(54670): n is a, c, g, or t
  misc_feature (54674)..(54674): n is a, c, g, or t
  misc_feature (54685)..(54685): n is a, c, g, or t
  misc_feature (54687)..(54687): n is a, c, g, or t
  misc_feature (54690)..(54690): n is a, c, g, or t
  misc_feature (54697)..(54697): n is a, c, g, or t
  misc_feature (54705)..(54705): n is a, c, g, or t
  misc_feature (54723)..(54723): n is a, c, g, or t
  misc_feature (54726)..(54726): n is a, c, g, or t
  misc_feature (54728)..(54728): n is a, c, g, or t
  misc_feature (54732)..(54733): n is a, c, g, or t
  misc_feature (54740)..(54741): n is a, c, g, or t
  misc_feature (54743)..(54743): n is a, c, g, or t
  misc_feature (54747)..(54747): n is a, c, g, or t
  misc_feature (54750)..(54750): n is a, c, g, or t
  misc_feature (54771)..(54771): n is a, c, g, or t
  misc_feature (54775)..(54775): n is a, c, g, or t
  misc_feature (54777)..(54777): n is a, c, g, or t
  misc_feature (54780)..(54780): n is a, c, g, or t
  misc_feature (54795)..(54798): n is a, c, g, or t
  misc_feature (54802)..(54802): n is a, c, g, or t
  misc_feature (54804)..(54804): n is a, c, g, or t
  misc_feature (54814)..(54814): n is a, c, g, or t
  misc_feature (54822)..(54822): n is a, c, g, or t
  misc_feature (54827)..(54827): n is a, c, g, or t
  misc_feature (54854)..(54855): n is a, c, g, or t
  misc_feature (54862)..(54862): n is a, c, g, or t
  misc_feature (54864)..(54864): n is a, c, g, or t
  misc_feature (54871)..(54871): n is a, c, g, or t
  misc_feature (54876)..(54876): n is a, c, g, or t
  misc_feature (54889)..(54889): n is a, c, g, or t
  misc_feature (54905)..(54905): n is a, c, g, or t
  misc_feature (54911)..(54912): n is a, c, g, or t
  misc_feature (54914)..(54914): n is a, c, g, or t
  misc_feature (54916)..(54916): n is a, c, g, or t
  misc_feature (54918)..(54918): n is a, c, g, or t
  misc_feature (54927)..(54927): n is a, c, g, or t
  misc_feature (54937)..(54937): n is a, c, g, or t
  misc_feature (54942)..(54942): n is a, c, g, or t
  misc_feature (54947)..(54947): n is a, c, g, or t
  misc_feature (54951)..(54951): n is a, c, g, or t
  misc_feature (54963)..(54963): n is a, c, g, or t
  misc_feature (54969)..(54969): n is a, c, g, or t
  misc_feature (54978)..(54980): n is a, c, g, or t
  misc_feature (54985)..(54987): n is a, c, g, or t
  misc_feature (54996)..(54996): n is a, c, g, or t
  misc_feature (55015)..(55015): n is a, c, g, or t
  misc_feature (55405)..(55405): n is a, c, g, or t
  misc_feature (55410)..(55410): n is a, c, g, or t
  misc_feature (55415)..(55415): n is a, c, g, or t
  misc_feature (55419)..(55419): n is a, c, g, or t
  misc_feature (55431)..(55431): n is a, c, g, or t
  misc_feature (55437)..(55437): n is a, c, g, or t
  misc_feature (55446)..(55448): n is a, c, g, or t
  misc_feature (55453)..(55455): n is a, c, g, or t
  misc_feature (55464)..(55464): n is a, c, g, or t
  misc_feature (55483)..(55483): n is a, c, g, or t
  misc_feature (55514)..(55514): n is a, c, g, or t
  misc_feature (55517)..(55517): n is a, c, g, or t
  misc_feature (55523)..(55523): n is a, c, g, or t
  misc_feature (55558)..(55558): n is a, c, g, or t
  misc_feature (55561)..(55561): n is a, c, g, or t
  misc_feature (55563)..(55563): n is a, c, g, or t
  misc_feature (55566)..(55566): n is a, c, g, or t
  misc_feature (55572)..(55572): n is a, c, g, or t
  misc_feature (55588)..(55588): n is a, c, g, or t
  misc_feature (55594)..(55594): n is a, c, g, or t
  misc_feature (55598)..(55598): n is a, c, g, or t
  misc_feature (55603)..(55603): n is a, c, g, or t
  misc_feature (55605)..(55606): n is a, c, g, or t
  misc_feature (55610)..(55610): n is a, c, g, or t
  misc_feature (55621)..(55621): n is a, c, g, or t
  misc_feature (55623)..(55623): n is a, c, g, or t
  misc_feature (55626)..(55626): n is a, c, g, or t
  misc_feature (55633)..(55633): n is a, c, g, or t
  misc_feature (55641)..(55641): n is a, c, g, or t
  misc_feature (55659)..(55659): n is a, c, g, or t
  misc_feature (55662)..(55662): n is a, c, g, or t
  misc_feature (55664)..(55664): n is a, c, g, or t
  misc_feature (55668)..(55669): n is a, c, g, or t
  misc_feature (55676)..(55677): n is a, c, g, or t
  misc_feature (55679)..(55679): n is a, c, g, or t
  misc_feature (55683)..(55683): n is a, c, g, or t
  misc_feature (55686)..(55686): n is a, c, g, or t
  misc_feature (55707)..(55707): n is a, c, g, or t
  misc_feature (55711)..(55711): n is a, c, g, or t
  misc_feature (55713)..(55713): n is a, c, g, or t
  misc_feature (55716)..(55716): n is a, c, g, or t
  misc_feature (55731)..(55734): n is a, c, g, or t
  misc_feature (55738)..(55738): n is a, c, g, or t
  misc_feature (55740)..(55740): n is a, c, g, or t
  misc_feature (55750)..(55750): n is a, c, g, or t
  misc_feature (55758)..(55758): n is a, c, g, or t
  misc_feature (55763)..(55763): n is a, c, g, or t
  misc_feature (55790)..(55791): n is a, c, g, or t
  misc_feature (55798)..(55798): n is a, c, g, or t
  misc_feature (55800)..(55800): n is a, c, g, or t
  misc_feature (55807)..(55807): n is a, c, g, or t
  misc_feature (55812)..(55812): n is a, c, g, or t
  misc_feature (55825)..(55825): n is a, c, g, or t
  misc_feature (55841)..(55841): n is a, c, g, or t
  misc_feature (55847)..(55848): n is a, c, g, or t
  misc_feature (55850)..(55850): n is a, c, g, or t
  misc_feature (55852)..(55852): n is a, c, g, or t
  misc_feature (55854)..(55854): n is a, c, g, or t
  misc_feature (55863)..(55863): n is a, c, g, or t
  misc_feature (55873)..(55873): n is a, c, g, or t
  misc_feature (55878)..(55878): n is a, c, g, or t
  misc_feature (55883)..(55883): n is a, c, g, or t
  misc_feature (55887)..(55887): n is a, c, g, or t
  misc_feature (55899)..(55899): n is a, c, g, or t
  misc_feature (55905)..(55905): n is a, c, g, or t
  misc_feature (55914)..(55916): n is a, c, g, or t
  misc_feature (55921)..(55923): n is a, c, g, or t
  misc_feature (55932)..(55932): n is a, c, g, or t
  misc_feature (55951)..(55951): n is a, c, g, or t
  misc_feature (56341)..(56341): n is a, c, g, or t
  misc_feature (56346)..(56346): n is a, c, g, or t
  misc_feature (56351)..(56351): n is a, c, g, or t
  misc_feature (56355)..(56355): n is a, c, g, or t
  misc_feature (56367)..(56367): n is a, c, g, or t
  misc_feature (56373)..(56373): n is a, c, g, or t
  misc_feature (56382)..(56384): n is a, c, g, or t
  misc_feature (56389)..(56391): n is a, c, g, or t
  misc_feature (56400)..(56400): n is a, c, g, or t
  misc_feature (56419)..(56419): n is a, c, g, or t
  misc_feature (56450)..(56450): n is a, c, g, or t
  misc_feature (56453)..(56453): n is a, c, g, or t
  misc_feature (56459)..(56459): n is a, c, g, or t
  misc_feature (56494)..(56494): n is a, c, g, or t
  misc_feature (56497)..(56497): n is a, c, g, or t
  misc_feature (56499)..(56499): n is a, c, g, or t
  misc_feature (56502)..(56502): n is a, c, g, or t
  misc_feature (56508)..(56508): n is a, c, g, or t
  misc_feature (56524)..(56524): n is a, c, g, or t
  misc_feature (56530)..(56530): n is a, c, g, or t
  misc_feature (56534)..(56534): n is a, c, g, or t
  misc_feature (56539)..(56539): n is a, c, g, or t
  misc_feature (56541)..(56542): n is a, c, g, or t
  misc_feature (56546)..(56546): n is a, c, g, or t
  misc_feature (56557)..(56557): n is a, c, g, or t
  misc_feature (56559)..(56559): n is a, c, g, or t
  misc_feature (56562)..(56562): n is a, c, g, or t
  misc_feature (56569)..(56569): n is a, c, g, or t
  misc_feature (56577)..(56577): n is a, c, g, or t
  misc_feature (56595)..(56595): n is a, c, g, or t
  misc_feature (56598)..(56598): n is a, c, g, or t
  misc_feature (56600)..(56600): n is a, c, g, or t
  misc_feature (56604)..(56605): n is a, c, g, or t
  misc_feature (56612)..(56613): n is a, c, g, or t
  misc_feature (56615)..(56615): n is a, c, g, or t
  misc_feature (56619)..(56619): n is a, c, g, or t
  misc_feature (56622)..(56622): n is a, c, g, or t
  misc_feature (56643)..(56643): n is a, c, g, or t
  misc_feature (56647)..(56647): n is a, c, g, or t
  misc_feature (56649)..(56649): n is a, c, g, or t
  misc_feature (56652)..(56652): n is a, c, g, or t
  misc_feature (56667)..(56670): n is a, c, g, or t
  misc_feature (56674)..(56674): n is a, c, g, or t
  misc_feature (56676)..(56676): n is a, c, g, or t
  misc_feature (56686)..(56686): n is a, c, g, or t
  misc_feature (56694)..(56694): n is a, c, g, or t
  misc_feature (56699)..(56699): n is a, c, g, or t
  misc_feature (56726)..(56727): n is a, c, g, or t
  misc_feature (56734)..(56734): n is a, c, g, or t
  misc_feature (56736)..(56736): n is a, c, g, or t
  misc_feature (56743)..(56743): n is a, c, g, or t
  misc_feature (56748)..(56748): n is a, c, g, or t
  misc_feature (56761)..(56761): n is a, c, g, or t
  misc_feature (56777)..(56777): n is a, c, g, or t
  misc_feature (56783)..(56784): n is a, c, g, or t
  misc_feature (56786)..(56786): n is a, c, g, or t
  misc_feature (56788)..(56788): n is a, c, g, or t
  misc_feature (56790)..(56790): n is a, c, g, or t
  misc_feature (56799)..(56799): n is a, c, g, or t
  misc_feature (56809)..(56809): n is a, c, g, or t
  misc_feature (56814)..(56814): n is a, c, g, or t
  misc_feature (56819)..(56819): n is a, c, g, or t
  misc_feature (56823)..(56823): n is a, c, g, or t
  misc_feature (56835)..(56835): n is a, c, g, or t
  misc_feature (56841)..(56841): n is a, c, g, or t
  misc_feature (56850)..(56852): n is a, c, g, or t
  misc_feature (56857)..(56859): n is a, c, g, or t
  misc_feature (56868)..(56868): n is a, c, g, or t
  misc_feature (56887)..(56887): n is a, c, g, or t
  misc_feature (57475)..(57475): n is a, c, g, or t
  misc_feature (57477)..(57478): n is a, c, g, or t
  misc_feature (57482)..(57482): n is a, c, g, or t
  misc_feature (57493)..(57493): n is a, c, g, or t
  misc_feature (57495)..(57495): n is a, c, g, or t
  misc_feature (57498)..(57498): n is a, c, g, or t
  misc_feature (57505)..(57505): n is a, c, g, or t
  misc_feature (57513)..(57513): n is a, c, g, or t
  misc_feature (57531)..(57531): n is a, c, g, or t
  misc_feature (57534)..(57534): n is a, c, g, or t
  misc_feature (57536)..(57536): n is a, c, g, or t
  misc_feature (57540)..(57541): n is a, c, g, or t
  misc_feature (57548)..(57549): n is a, c, g, or t
  misc_feature (57551)..(57551): n is a, c, g, or t
  misc_feature (57555)..(57555): n is a, c, g, or t
  misc_feature (57558)..(57558): n is a, c, g, or t
  misc_feature (57579)..(57579): n is a, c, g, or t
  misc_feature (57583)..(57583): n is a, c, g, or t
  misc_feature (57585)..(57585): n is a, c, g, or t
  misc_feature (57588)..(57588): n is a, c, g, or t
  misc_feature (57603)..(57606): n is a, c, g, or t
  misc_feature (57610)..(57610): n is a, c, g, or t
  misc_feature (57612)..(57612): n is a, c, g, or t
  misc_feature (57622)..(57622): n is a, c, g, or t
  misc_feature (57630)..(57630): n is a, c, g, or t
  misc_feature (57635)..(57635): n is a, c, g, or t
  misc_feature (57662)..(57663): n is a, c, g, or t
  misc_feature (57670)..(57670): n is a, c, g, or t
  misc_feature (57672)..(57672): n is a, c, g, or t
  misc_feature (57679)..(57679): n is a, c, g, or t
  misc_feature (57684)..(57684): n is a, c, g, or t
  misc_feature (57697)..(57697): n is a, c, g, or t
  misc_feature (57713)..(57713): n is a, c, g, or t
  misc_feature (57719)..(57720): n is a, c, g, or t
  misc_feature (57722)..(57722): n is a, c, g, or t
  misc_feature (57724)..(57724): n is a, c, g, or t
  misc_feature (57726)..(57726): n is a, c, g, or t
  misc_feature (57735)..(57735): n is a, c, g, or t
  misc_feature (57745)..(57745): n is a, c, g, or t
  misc_feature (57750)..(57750): n is a, c, g, or t
  misc_feature (57755)..(57755): n is a, c, g, or t
  misc_feature (57759)..(57759): n is a, c, g, or t
  misc_feature (57771)..(57771): n is a, c, g, or t
  misc_feature (57777)..(57777): n is a, c, g, or t
  misc_feature (57786)..(57788): n is a, c, g, or t
  misc_feature (57793)..(57795): n is a, c, g, or t
  misc_feature (57804)..(57804): n is a, c, g, or t
  misc_feature (57823)..(57823): n is a, c, g, or t
  misc_feature (58213)..(58213): n is a, c, g, or t
  misc_feature (58218)..(58218): n is a, c, g, or t
  misc_feature (58223)..(58223): n is a, c, g, or t
  misc_feature (58227)..(58227): n is a, c, g, or t
  misc_feature (58239)..(58239): n is a, c, g, or t
  misc_feature (58245)..(58245): n is a, c, g, or t
  misc_feature (58254)..(58256): n is a, c, g, or t
  misc_feature (58261)..(58263): n is a, c, g, or t
  misc_feature (58272)..(58272): n is a, c, g, or t
  misc_feature (58291)..(58291): n is a, c, g, or t
  misc_feature (58322)..(58322): n is a, c, g, or t
  misc_feature (58325)..(58325): n is a, c, g, or t
  misc_feature (58331)..(58331): n is a, c, g, or t
  misc_feature (58366)..(58366): n is a, c, g, or t
  misc_feature (58369)..(58369): n is a, c, g, or t
  misc_feature (58371)..(58371): n is a, c, g, or t
  misc_feature (58374)..(58374): n is a, c, g, or t
  misc_feature (58380)..(58380): n is a, c, g, or t
  misc_feature (58396)..(58396): n is a, c, g, or t
  misc_feature (58402)..(58402): n is a, c, g, or t
  misc_feature (58406)..(58406): n is a, c, g, or t
  misc_feature (58411)..(58411): n is a, c, g, or t
  misc_feature (58413)..(58414): n is a, c, g, or t
  misc_feature (58418)..(58418): n is a, c, g, or t
  misc_feature (58429)..(58429): n is a, c, g, or t
  misc_feature (58431)..(58431): n is a, c, g, or t
  misc_feature (58434)..(58434): n is a, c, g, or t
  misc_feature (58441)..(58441): n is a, c, g, or t
  misc_feature (58449)..(58449): n is a, c, g, or t
  misc_feature (58467)..(58467): n is a, c, g, or t
  misc_feature (58470)..(58470): n is a, c, g, or t
  misc_feature (58472)..(58472): n is a, c, g, or t
  misc_feature (58476)..(58477): n is a, c, g, or t
  misc_feature (58484)..(58485): n is a, c, g, or t
  misc_feature (58487)..(58487): n is a, c, g, or t
  misc_feature (58491)..(58491): n is a, c, g, or t
  misc_feature (58494)..(58494): n is a, c, g, or t
  misc_feature (58515)..(58515): n is a, c, g, or t
  misc_feature (58519)..(58519): n is a, c, g, or t
  misc_feature (58521)..(58521): n is a, c, g, or t
  misc_feature (58524)..(58524): n is a, c, g, or t
  misc_feature (58539)..(58542): n is a, c, g, or t
  misc_feature (58546)..(58546): n is a, c, g, or t
  misc_feature (58548)..(58548): n is a, c, g, or t
  misc_feature (58558)..(58558): n is a, c, g, or t
  misc_feature (58566)..(58566): n is a, c, g, or t
  misc_feature (58571)..(58571): n is a, c, g, or t
  misc_feature (58598)..(58599): n is a, c, g, or t
  misc_feature (58606)..(58606): n is a, c, g, or t
  misc_feature (58608)..(58608): n is a, c, g, or t
  misc_feature (58615)..(58615): n is a, c, g, or t
  misc_feature (58620)..(58620): n is a, c, g, or t
  misc_feature (58633)..(58633): n is a, c, g, or t
  misc_feature (58649)..(58649): n is a, c, g, or t
  misc_feature (58655)..(58656): n is a, c, g, or t
  misc_feature (58658)..(58658): n is a, c, g, or t
  misc_feature (58660)..(58660): n is a, c, g, or t
  misc_feature (58662)..(58662): n is a, c, g, or t
  misc_feature (58671)..(58671): n is a, c, g, or t
  misc_feature (58681)..(58681): n is a, c, g, or t
  misc_feature (58686)..(58686): n is a, c, g, or t
  misc_feature (58691)..(58691): n is a, c, g, or t
  misc_feature (58695)..(58695): n is a, c, g, or t
  misc_feature (58707)..(58707): n is a, c, g, or t
  misc_feature (58713)..(58713): n is a, c, g, or t
  misc_feature (58722)..(58724): n is a, c, g, or t
  misc_feature (58729)..(58731): n is a, c, g, or t
  misc_feature (58740)..(58740): n is a, c, g, or t
  misc_feature (58759)..(58759): n is a, c, g, or t
  misc_feature (59149)..(59149): n is a, c, g, or t
  misc_feature (59154)..(59154): n is a, c, g, or t
  misc_feature (59159)..(59159): n is a, c, g, or t
  misc_feature (59163)..(59163): n is a, c, g, or t
  misc_feature (59175)..(59175): n is a, c, g, or t
  misc_feature (59181)..(59181): n is a, c, g, or t
  misc_feature (59190)..(59192): n is a, c, g, or t
  misc_feature (59197)..(59199): n is a, c, g, or t
  misc_feature (59208)..(59208): n is a, c, g, or t
  misc_feature (59227)..(59227): n is a, c, g, or t
  misc_feature (59258)..(59258): n is a, c, g, or t
  misc_feature (59261)..(59261): n is a, c, g, or t
  misc_feature (59267)..(59267): n is a, c, g, or t
  misc_feature (59302)..(59302): n is a, c, g, or t
  misc_feature (59305)..(59305): n is a, c, g, or t
  misc_feature (59307)..(59307): n is a, c, g, or t
  misc_feature (59310)..(59310): n is a, c, g, or t
  misc_feature (59316)..(59316): n is a, c, g, or t
  misc_feature (59332)..(59332): n is a, c, g, or t
  misc_feature (59338)..(59338): n is a, c, g, or t
  misc_feature (59342)..(59342): n is a, c, g, or t
  misc_feature (59347)..(59347): n is a, c, g, or t
  misc_feature (59349)..(59350): n is a, c, g, or t
  misc_feature (59354)..(59354): n is a, c, g, or t
  misc_feature (59365)..(59365): n is a, c, g, or t
  misc_feature (59367)..(59367): n is a, c, g, or t
  misc_feature (59370)..(59370): n is a, c, g, or t
  misc_feature (59377)..(59377): n is a, c, g, or t
  misc_feature (59385)..(59385): n is a, c, g, or t
  misc_feature (59403)..(59403): n is a, c, g, or t
  misc_feature (59406)..(59406): n is a, c, g, or t
  misc_feature (59408)..(59408): n is a, c, g, or t
  misc_feature (59412)..(59413): n is a, c, g, or t
  misc_feature (59420)..(59421): n is a, c, g, or t
  misc_feature (59423)..(59423): n is a, c, g, or t
  misc_feature (59427)..(59427): n is a, c, g, or t
  misc_feature (59430)..(59430): n is a, c, g, or t
  misc_feature (59451)..(59451): n is a, c, g, or t
  misc_feature (59455)..(59455): n is a, c, g, or t
  misc_feature (59457)..(59457): n is a, c, g, or t
  misc_feature (59460)..(59460): n is a, c, g, or t
  misc_feature (59475)..(59478): n is a, c, g, or t
  misc_feature (59482)..(59482): n is a, c, g, or t
  misc_feature (59484)..(59484): n is a, c, g, or t
  misc_feature (59494)..(59494): n is a, c, g, or t
  misc_feature (59502)..(59502): n is a, c, g, or t
  misc_feature (59507)..(59507): n is a, c, g, or t
  misc_feature (59534)..(59535): n is a, c, g, or t
  misc_feature (59542)..(59542): n is a, c, g, or t
  misc_feature (59544)..(59544): n is a, c, g, or t
  misc_feature (59551)..(59551): n is a, c, g, or t
  misc_feature (59556)..(59556): n is a, c, g, or t
  misc_feature (59569)..(59569): n is a, c, g, or t
  misc_feature (59585)..(59585): n is a, c, g, or t
  misc_feature (59591)..(59592): n is a, c, g, or t
  misc_feature (59594)..(59594): n is a, c, g, or t
  misc_feature (59596)..(59596): n is a, c, g, or t
  misc_feature (59598)..(59598): n is a, c, g, or t
  misc_feature (59607)..(59607): n is a, c, g, or t
  misc_feature (59617)..(59617): n is a, c, g, or t
  misc_feature (59622)..(59622): n is a, c, g, or t
  misc_feature (59627)..(59627): n is a, c, g, or t
  misc_feature (59631)..(59631): n is a, c, g, or t
  misc_feature (59643)..(59643): n is a, c, g, or t
  misc_feature (59649)..(59649): n is a, c, g, or t
  misc_feature (59658)..(59660): n is a, c, g, or t
  misc_feature (59665)..(59667): n is a, c, g, or t
  misc_feature (59676)..(59676): n is a, c, g, or t
  misc_feature (59695)..(59695): n is a, c, g, or t
  misc_feature (60082)..(60082): n is a, c, g, or t
  misc_feature (60087)..(60087): n is a, c, g, or t
  misc_feature (60092)..(60092): n is a, c, g, or t
  misc_feature (60096)..(60096): n is a, c, g, or t
  misc_feature (60108)..(60108): n is a, c, g, or t
  misc_feature (60114)..(60114): n is a, c, g, or t
  misc_feature (60123)..(60125): n is a, c, g, or t
  misc_feature (60130)..(60132): n is a, c, g, or t
  misc_feature (60141)..(60141): n is a, c, g, or t
  misc_feature (60160)..(60160): n is a, c, g, or t
  misc_feature (60191)..(60191): n is a, c, g, or t
  misc_feature (60194)..(60194): n is a, c, g, or t
  misc_feature (60200)..(60200): n is a, c, g, or t
  misc_feature (60235)..(60235): n is a, c, g, or t
  misc_feature (60238)..(60238): n is a, c, g, or t
  misc_feature (60240)..(60240): n is a, c, g, or t
  misc_feature (60243)..(60243): n is a, c, g, or t
  misc_feature (60249)..(60249): n is a, c, g, or t
  misc_feature (60265)..(60265): n is a, c, g, or t
  misc_feature (60271)..(60271): n is a, c, g, or t
  misc_feature (60275)..(60275): n is a, c, g, or t
  misc_feature (60280)..(60280): n is a, c, g, or t
  misc_feature (60282)..(60283): n is a, c, g, or t
  misc_feature (60287)..(60287): n is a, c, g, or t
  misc_feature (60298)..(60298): n is a, c, g, or t
  misc_feature (60300)..(60300): n is a, c, g, or t
  misc_feature (60303)..(60303): n is a, c, g, or t
  misc_feature (60310)..(60310): n is a, c, g, or t
  misc_feature (60318)..(60318): n is a, c, g, or t
  misc_feature (60336)..(60336): n is a, c, g, or t
  misc_feature (60339)..(60339): n is a, c, g, or t
  misc_feature (60341)..(60341): n is a, c, g, or t
  misc_feature (60345)..(60346): n is a, c, g, or t
  misc_feature (60353)..(60354): n is a, c, g, or t
  misc_feature (60356)..(60356): n is a, c, g, or t
  misc_feature (60360)..(60360): n is a, c, g, or t
  misc_feature (60363)..(60363): n is a, c, g, or t
  misc_feature (60384)..(60384): n is a, c, g, or t
  misc_feature (60388)..(60388): n is a, c, g, or t
  misc_feature (60390)..(60390): n is a, c, g, or t
  misc_feature (60393)..(60393): n is a, c, g, or t
  misc_feature (60408)..(60411): n is a, c, g, or t
  misc_feature (60415)..(60415): n is a, c, g, or t
  misc_feature (60417)..(60417): n is a, c, g, or t
  misc_feature (60427)..(60427): n is a, c, g, or t
  misc_feature (60435)..(60435): n is a, c, g, or t
  misc_feature (60440)..(60440): n is a, c, g, or t
  misc_feature (60467)..(60468): n is a, c, g, or t
  misc_feature (60475)..(60475): n is a, c, g, or t
  misc_feature (60477)..(60477): n is a, c, g, or t
  misc_feature (60484)..(60484): n is a, c, g, or t
  misc_feature (60489)..(60489): n is a, c, g, or t
  misc_feature (60502)..(60502): n is a, c, g, or t
  misc_feature (60518)..(60518): n is a, c, g, or t
  misc_feature (60524)..(60525): n is a, c, g, or t
  misc_feature (60527)..(60527): n is a, c, g, or t
  misc_feature (60529)..(60529): n is a, c, g, or t
  misc_feature (60531)..(60531): n is a, c, g, or t
  misc_feature (60540)..(60540): n is a, c, g, or t
  misc_feature (60550)..(60550): n is a, c, g, or t
  misc_feature (60555)..(60555): n is a, c, g, or t
  misc_feature (60560)..(60560): n is a, c, g, or t
  misc_feature (60564)..(60564): n is a, c, g, or t
  misc_feature (60576)..(60576): n is a, c, g, or t
  misc_feature (60582)..(60582): n is a, c, g, or t
  misc_feature (60591)..(60593): n is a, c, g, or t
  misc_feature (60598)..(60600): n is a, c, g, or t
  misc_feature (60609)..(60609): n is a, c, g, or t
  misc_feature (60628)..(60628): n is a, c, g, or t
  misc_feature (61018)..(61018): n is a, c, g, or t
  misc_feature (61023)..(61023): n is a, c, g, or t
  misc_feature (61028)..(61028): n is a, c, g, or t
  misc_feature (61032)..(61032): n is a, c, g, or t
  misc_feature (61044)..(61044): n is a, c, g, or t
  misc_feature (61050)..(61050): n is a, c, g, or t
  misc_feature (61059)..(61061): n is a, c, g, or t
  misc_feature (61066)..(61068): n is a, c, g, or t
  misc_feature (61077)..(61077): n is a, c, g, or t
  misc_feature (61096)..(61096): n is a, c, g, or t
  misc_feature (61127)..(61127): n is a, c, g, or t
  misc_feature (61130)..(61130): n is a, c, g, or t
  misc_feature (61136)..(61136): n is a, c, g, or t
  misc_feature (61171)..(61171): n is a, c, g, or t
  misc_feature (61174)..(61174): n is a, c, g, or t
  misc_feature (61176)..(61176): n is a, c, g, or t
  misc_feature (61179)..(61179): n is a, c, g, or t
  misc_feature (61185)..(61185): n is a, c, g, or t
  misc_feature (61201)..(61201): n is a, c, g, or t
  misc_feature (61207)..(61207): n is a, c, g, or t
  misc_feature (61211)..(61211): n is a, c, g, or t
  misc_feature (62620)..(62620): n is a, c, g, or t
  misc_feature (62622)..(62623): n is a, c, g, or t
  misc_feature (62627)..(62627): n is a, c, g, or t
  misc_feature (62638)..(62638): n is a, c, g, or t
  misc_feature (62640)..(62640): n is a, c, g, or t
  misc_feature (62643)..(62643): n is a, c, g, or t
  misc_feature (62650)..(62650): n is a, c, g, or t
  misc_feature (62658)..(62658): n is a, c, g, or t
  misc_feature (62676)..(62676): n is a, c, g, or t
  misc_feature (62679)..(62679): n is a, c, g, or t
  misc_feature (62681)..(62681): n is a, c, g, or t
  misc_feature (62685)..(62686): n is a, c, g, or t
  misc_feature (62693)..(62694): n is a, c, g, or t
  misc_feature (62696)..(62696): n is a, c, g, or t
  misc_feature (62700)..(62700): n is a, c, g, or t
  misc_feature (62703)..(62703): n is a, c, g, or t
  misc_feature (62724)..(62724): n is a, c, g, or t
  ref <308>: DATABASE ACCESSION NUMBER: GenBank / AF414442
  ref <309>: DATABASE ENTRY DATE: 2002-10-29
  ref <313>: RELEVANT RESIDUES: (1)..(66765)
aagcgttgcacaattcccccaacctccatacatacggcagctcttctagacacaggttttcccaggtcaaatgcggggaccccagccatatctcccaccctgagaaattttggagtttcagggagctcagaagctctgcagaggccaccctctctgaggggattcttcttagacctccatccagaggcaaatgttgacctgtccatgctgaaaccctcaggccttcctgggtcatcttctcccacccgctccttgatgacagggagcaggagcactaaagccacaccagaaatggattcaggactgacaggagccaccttgtcacctaagacatctacaggtgcaatcgtggtgacagaacatactctgccctttacttccccagataagaccttggccagtcctacatcttcggttgtgggaagaaccacccagtctttgggggtgatgtcctctgctctccctgagtcaacctctagaggaatgacacactccgagcaaagaaccagcccatcgctgagtccccaggtcaatggaactccctctaggaactaccctgctacaagcatggtttcaggattgagttccccaaggaccaggaccagttccacagaaggaaattttaccaaagaagcatctacatacacactcactgtagagaccacaagtggcccagtcactgagaagtacacagtccccactgagacctcaacaactgaaggtgacagcacagagaccccctgggacacaagatatattcctgtaaaaatcacatctccaatgaaaacatttgcagattcaactgcatccaaggaaaatgccccagtgtctatgactccagctgagaccacagttactgactcacatactccaggaaggacaaacccatcatttgggacactttattcttccttccttgacctatcacctaaagggaccccaaattccagaggtgaaacaagcctggaactgattctatcaaccactggatatcccttctcctctcctgaacctggctctgcaggacacagcagaataagtaccagtgcgcctttgtcatcatctgcttcagttctcgataataaaatatcagagaccagcatattctcaggccagagtctcacctcccctctgtctcctggggtgcccgaggccagagccagcacaatgcccaactcagctatccctttttccatgacactaagcaatgcagaaacaagtgccgaaagggtcagaagcacaatttcctctctggggactccatcaatatccacaaagcagacagcagagactatccttaccttccatgccttcgctgagaccatggatatacccagcacccacatagccaagactttggcttcagaatggttgggaagtccaggtacccttggtggcaccagcacttcagcgctgacaaccacatctccatctaccactttagtctcagaggagaccaacacccatcactccacgagtggaaaggaaacagaaggaactttgaatacatctatgactccacttgagacctctgctcctggagaagagtccgaaatgactgccaccttggtccccactctaggttttacaactcttgacagcaagatcagaagtccatctcaggtctcttcatcccacccaacaagagagctcagaaccacaggcagcacctctgggaggcagagttccagcacagctgcccacgggagctctgacatcctgagggcaaccacttccagcacctcaaaagcatcatcatggaccagtgaaagcacagctcagcaatttagtgaaccccagcacacacagtgggtggagacaagtcctagcatgaaaacagagagacccccagcatcaaccagtgtggcagcccctatcaccacttctgttccctcagtggtctctggcttcaccaccctgaagaccagctccacaaaagggatttggcttgaagaaacatctgcagacacactcatcggagaatccacagctggcccaaccacccatcagtttgctgttcccactgggatttcaatgacaggaggcagcagcaccaggggaagccagggcacaacccacctactcaccagagccacagcatcatctgagacatccgcagatttgactctggccacgaacggtgtcccagtctccgtgtctccagcagtgagcaagacggctgctggctcaagtcctccaggagggacaaagccatcatatacaatggtttcttctgtcatccctgagacatcatctctacagtcctcagctttcagggaaggaaccagcctgggactgactccattaaacactagacatcccttctcttcccctgaaccagactctgcaggacacaccaagataagcaccagcattcctctgttgtcatctgcttcagttcttgaggataaagtgtcagcgaccagcacattctcacaccacaaagccacctcatctattaccacagggactcctgaaatctcaacaaagacaaagcccagctcagccgttctttcctccatgaccctaagcaatgcagcaacaagtcctgaaagagtcagaaatgcaacttcccctctgactcatccatctccatcaggggaagagacagcagggagtgtcctcactctcagcacctctgctgagactacagactcacctaacatccacccaactgggacactgacttcagaatcgtcagagagtcctagcactctcagcctcccaagtgtctctggagtcaaaaccacattttcttcatctactccttccactcatctatttactagtggagaagaaacagaggaaacttcgaatccatctgtgtctcaacctgagacttctgtttccagagtaaggaccaccttggccagcacctctgtccctaccccagtattccccaccatggacacctggcctacacgttcagctcagttctcttcatcccacctagtgagtgagctcagagctacgagcagtacctcagttacaaactcaactggttcagctcttcctaaaatatctcacctcactgggacggcaacaatgtcacagaccaatagagacacgtttaatgactctgctgcaccccaaagcacaacttggccagagactagtcccagattcaagacagggttaccttcagcaacaaccactgtttcaacctctgccacttctctctctgctactgtaatggtctctaaattcacttctccagcaactagttccatggaagcaacttctatcagggaaccatcaacaaccatcctcacaacagagaccacgaatggcccaggctctatggctgtggcttctaccaacatcccaattggaaagggctacattactgaaggaagattggacacaagccatctgcccattggaaccacagcttcctctgagacatctatggattttaccatggccaaagaaagtgtctcaatgtcagtatctccatctcagtccatggatgctgctggctcaagcactccaggaaggacaagccaattcgttgacacattttctgatgatgtctatcatttaacatccagagaaattacaatacctagagatggaacaagctcagctctgactccacaaatgactgcaactcaccctccatctcctgatcctggctctgctagaagcacctggcttggcatcttgtcctcatctccttcttctcctactcccaaagtcacaatgagctccacattttcaactcagagagtcaccacaagcatgataatggacacagttgaaactagtcggtggaacatgcccaacttaccttccacgacttccctgacaccaagtaatattccaacaagtggtgccataggaaaaagcaccctggttcccttggacactccatctccagccacatcattggaggcatcagaagggggacttccaaccctcagcacctaccctgaatcaacaaacacacccagcatccacctcggagcacacgctagttcagaaagtccaagcaccatcaaacttaccatggcttcagtagtaaaacctggctcttacacacctctcaccttcccctcaatagagacccacattcatgtatcaacagccagaatggcttactcttctgggtcttcacctgagatgacagctcctggagagactaacactggtagtacctgggaccccaccacctacatcaccactacggatcctaaggatacaagttcagctcaggtctctacaccccactcagtgaggacactcagaaccacagaaaaccatccaaagacagagtccgccaccccagctgcttactctggaagtcctaaaatctcaagttcacccaatctcaccagtccggccacaaaagcatggaccatcacagacacaactgaacactccactcaattacattacacaaaattggcagaaaaatcatctggatttgagacacagtcagctccaggacctgtctctgtagtaatccctacctcccctaccattggaagcagcacattggaactaacttctgatgtcccaggggaacccctggtccttgctcccagtgagcagaccacaatcactctccccatggcaacatggctgagtaccagtttgacagaggaaatggcttcaacagaccttgatatttcaagtccaagttcacccatgagtacatttgctatttttccacctatgtccacaccttctcatgaactttcaaagtcagaggcagataccagtgccattagaaatacagattcaacaacgttggatcagcacctaggaatcaggagtttgggcagaactggggacttaacaactgttcctatcaccccactgacaaccacgtggaccagtgtgattgaacactcaacacaagcacaggacaccctttctgcaacgatgagtcctactcacgtgacacagtcactcaaagatcaaacatctataccagcctcagcatccccttcccatcttactgaagtctaccctgagctcgggacacaagggagaagctcctctgaggcaaccactttttggaaaccatctacagacacactgtccagagagattgagactggcccaacaaacattcaatccactccacccatggacaacacaacaacagggagcagtagtagtggagtcaccctgggcatagcccaccttcccataggaacatcctccccagctgagacatccacaaacatggcactggaaagaagaagttctacagccactgtctctatggctgggacaatgggactccttgttactagtgctccaggaagaagcatcagccagtcattaggaagagtttcctctgtcctttctgagtcaactactgaaggagtcacagattctagtaagggaagcagcccaaggctgaacacacagggaaatacagctctctcctcctctcttgaacccagctatgctgaaggaagccagatgagcacaagcatccctctaacctcatctcctacaactcctgatgtggaattcatagggggcagcacattttggaccaaggaggtcaccacagttatgacctcagacatctccaagtcttcagcaaggacagagtccagctcagctacccttatgtccacagctttgggaagcactgaaaatacaggaaaagaaaaactcagaactgcctctatggatcttccatctccaactccatcaatggaggtgacaccatggatttctctcactctcagtaatgcccccaataccacagattcacttgacctcagccatggggtgcacaccagctctgcagggactttggccactgacaggtcattgaatactggtgtcactagagcctccagattggaaaacggctctgatacctcttctaagtccctgtctatgggaaacagcactcacacttccatgactgacacagagaagagtgaagtgtcttcttcaatccatccccgacctgagacctcagctcctggagcagagaccactttgacttccactcctggaaacagggccataagcttaacattgcctttttcatccattccagtggaagaagtcatttctacaggcataacctcaggaccagacatcaactcagcacccatgacacattctcccatcaccccaccaacaattgtatggaccagtacaggcacaattgaacagtccactcaaccactacatgcagtttcttcagaaaaagtttctgtgcagacacagtcaactccatatgtcaactctgtggcagtgtctgcttcccctacccatgagaattcagtctcttctggaagcagcacatcctctccatattcctcagcctcacttgaatccttggattccacaatcagtaggaggaatgcaatcacttcctggctatgggacctcactacatctctccccactacaacttggccaagtactagtttatctgaggcactgtcctcaggccattctggggtttcaaacccaagttcaactacgactgaatttccactcttttcagctgcatccacatctgctgctaagcaaagaaatccagaaacagagacccatggtccccagaatacagccgcgagtactttgaacactgatgcatcctcggtcacaggtctttctgagactcctgtgggggcaagtatcagctctgaagtccctcttccaatggccataacttctagatcagatgtttctggccttacatctgagagtactgctaacccgagtttaggcacagcctcttcagcagggaccaaattaactaggacaatatccctgcccacttcagagtctttggtttcctttagaatgaacaaggatccatggacagtgtcaatccctttggggtcccatccaactactaatacagaaacaagcatcccagtaaacagcgcaggtccacctggcttgtccacagtagcatcagatgtaattgacacaccttcagatggggctgagagtattcccactgtctccttttccccctcccctgatactgaagtgacaactatctcacatttcccagaaaagacaactcattcatttagaaccatttcatctctcactcatgagttgacttcaagagtgacacctattcctggggattggatgagttcagctatgtctacaaagcccacaggagccagtccctccattacactgggagagagaaggacaatcacctctgctgctccaaccacttcccccatagttctcactgctagtttcacagagaccagcacagtttcactggataatgaaactacagtaaaaacctcagatatccttgacgcacggaaaacaaatgagctcccctcagatagcagttcttcttctgatctgatcaacacctccatagcttcttcaactatggatgtcactaaaacagcctccatcagtcccactagcatctcaggaatgacagcaagttcctccccatctctcttctcttcagatagaccccaggttcccacatctacaacagagacaaatacagccacctctccatctgtttccagtaacacctattctcttgatgggggctccaatgtgggtggcactccatccactttaccaccctttacaatcacccaccctgtcgagacaagctcggccctattagcctggtctagaccagtaagaactttcagcaccatggtcagcactgacactgcctccggagaaaatcctacctctagcaattctgtggtgacttctgttccagcaccaggtacatgggccagtgtaggcagtactactgacttacctgccatgggctttctcaagacaagtcctgcaggagaggcacactcacttctagcatcaactattgaaccagccactgccttcactccccatctctcagcagcagtggtcactggatccagtgctacatcagaagccagtcttctcactacgagtgaaagcaaagccattcattcttcaccacagaccccaactacacccacctctggagcaaactgggaaacttcagctactcctgagagccttttggtagtcactgagacttcagacacaacacttacctcaaagattttggtcacagataccatcttgttttcaactgtgtccacgccaccttctaaatttccaagtacggggactctgtctggagcttccttccctactttactcccggacactccagccatccctctcactgccactgagccaacaagttcattagctacatcctttgattccaccccactggtgactatagcttcggatagtcttggcacagtcccagagactaccctgaccatgtcagagacctcaaatggtgatgcactggttcttaagacagtaagtaacccagataggagcatccctggaatcactatccaaggagtaacagaaagtccactccatccttcttccacttccccctctaagattgttgctccacggaatacaacctatgaaggttcgatcacagtggcactttctactttgcctgcgggaactactggttcccttgtattcagtcagagttctgaaaactcagagacaacggctttggtagactcatcagctgggcttgagagggcatctgtgatgccactaaccacaggaagccagggtatggctagctctggaggaatcagaagtgggtccactcactcaactggaaccaaaacattttcttctctccctctgaccatgaacccaggtgaggttacagccatgtctgaaatcaccacgaacagactgacagctactcaatcaacagcacccaaagggatacctgtgaagcccaccagtgctgagtcaggcctcctaacacctgtctctgcctcctcaagcccatcaaaggcctttgcctcactgactacagctcccccatcaacttgggggatcccacagtctaccttgacatttgagttttctgaggtcccaagtttggatactaagtccgcttctttaccaactcctggacagtccctgaacaccattccagactcagatgcaagcacagcatcttcctcactgtccaagtctccagaaaaaaacccaagggcaaggatgatgacttccacaaaggccataagtgcaagctcatttcaatcaacaggttttactgaaacccctgagggatctgcctccccttctatggcagggcatgaacccagagtccccacttcaggaacaggggaccctagatatgcctcagagagcatgtcttatccagacccaagcaaggcatcatcagctatgacatcgacctctcttgcatcaaaactcacaactctcttcagcacaggtcaagcagcaaggtctggttctagttcctctcccataagcctatccactgagaaagaaacaagcttcctttcccccactgcatccacctccagaaagacttcactatttcttgggccttccatggcaaggcagcccaacatattggtgcatcttcagacttcagctctgacactttctccaacatccactctaaatatgtcccaggaggagcctcctgagttaacctcaagccagaccattgcagaagaagagggaacaacagctgaaacacagacgttaaccttcacaccatctgagaccccaacatccttgttacctgtctcttctcccacagaacccacagccagaagaaagagttctccagaaacatgggcaagctctatttcagttcctgccaagacctccttggttgaaacaactgatggaacgctagtgaccaccataaagatgtcaagccaggcagcacaaggaaattccacgtggcctgccccagcagaggagacggggaccagtccagcaggcacatccccaggaagcccagaagtgtctaccactctcaaaatcatgagctccaaggaacccagcatcagcccagagatcaggtccactgtgcgaaattctccttggaagactccagaaacaactgttcccatggagaccacagtggaaccagtcacccttcagtccacagccctaggaagtggcagcaccagcatctctcacctgcccacaggaaccacatcaccaaccaagtcaccaacagaaaatatgttggctacagaaagggtctccctctccccatccccacctgaggcttggaccaacctttattctggaactccaggagggaccaggcagtcactggccacaatgtcctctgtctccctagagtcaccaactgctagaagcatcacagggactggtcagcaaagcagtccagaactggtttcaaagacaactggaatggaattctctatgtggcatggctctactggagggaccacaggggacacacatgtctctctgagcacatcttccaatatccttgaagaccctgtaaccagcccaaactctgtgagctcattgacagataaatccaaacataaaaccgagacatgggtaagcaccacagccattccctccactgtcctgaataataagataatggcagctgaacaacagacaagtcgatctgtggatgaggcttattcatcaactagttcttggtcagatcagacatctgggagtgacatcacccttggtgcatctcctgatgtcacaaacacattatacatcacctccacagcacaaaccacctcactagtgtctctgccctctggagaccaaggcattacaagcctcaccaatccctcaggaggaaaaacaagctctgcgtcatctgtcacatctccttcaatagggcttgagactctgagggccaatgtaagtgcagtgaaaagtgacattgcccctactgctgggcatctatctcagacttcatctcctgcggaagtgagcatcctggacgtaaccacagctcctactccaggtatctccaccaccatcaccaccatgggaaccaactcaatctcaactaccacacccaacccagaagtgggtatgagtaccatggacagcaccccggccacagagaggcgcacaacttctacagaacacccttccacctggtcttccacagctgcatcagattcctggactgtcacagacatgacttcaaacttgaaagttgcaagatctcctggaacaatttccacaatgcatacaacttcattcttagcctcaagcactgaattagactccatgtctactccccatggccgtataactgtcattggaaccagcctggtcactccatcctctgatgcttcagctgtaaagacagagaccagtacaagtgaaagaacattgagtccttcagacacaactgcatctactcccatctcaactttttctcgtgtccagaggatgagcatctcagttcctgacattttaagtacaagttggactcccagtagtacagaagcagaagatgtgcctgtttcaatggtttctacagatcatgctagtacaaagactgacccaaatacgcccctgtccacttttctgtttgattctctgtccactcttgactgggacactgggagatctctgtcatcagccacagccactacctcagctcctcagggggccacaactccccaggaactcactttggaaaccatgatcagcccagctacctcacagttgcccttctctatagggcacattacaagtgcagtcacaccagctgcaatggcaaggagctctggagttactttttcaagaccagatcccacaagcaaaaaggcagagcagacttccactcagcttcccaccaccacttctgcacatccagggcaggtgcccagatcagcagcaacaactctggatgtgatcccacacacagcaaaaactccagatgcaacttttcagagacaagggcagacagctcttacaacagaggcaagagctacatctgactcctggaatgagaaagaaaaatcaaccccaagtgcaccttggatcactgagatgatgaattctgtctcagaagataccatcaaggaggttaccagctcctccagtgtattaaaggaccctgaatacgctggacataaacttggaatctgggacgacttcatccccaagtttggaaaagcagcccatatgagagagttgccccttctgagtccaccacaggacaaagaggcaattcacccttctacaaacacagtagagaccacaggctgggtcacaagttccgaacatgcttctcattccactatcccagcccactcagcgtcatccaaactcacatctccagtggttacaacctccaccagggaacaagcaatagtttctatgtcaacaaccacatggccagagtctacaagggctagaacagagcctaattccttcttgactattgaactgagggacgtcagcccttacatggacaccagctcaaccacacaaacaagtattatctcttccccaggttccactgcgatcaccaaggggcctagaacagaaattacctcctctaagagaatatccagctcattccttgcccagtctatgaggtcgtcagacagcccctcagaagccatcaccaggctgtctaactttcctgccatgacagaatctggaggaatgatccttgctatgcaaacaagtccacctggcgctacatcactaagtgcacctactttggatacatcagccacagcctcctggacagggactccactggctacgactcagagatttacatactcagagaagaccactctctttagcaaaggtcctgaggatacatcacagccaagccctccctctgtggaagaaaccagctcttcctcttccctggtacctatccatgctacaacctcgccttccaatattttgttgacatcacaagggcacagtccctcctctactccacctgtgacctcagttttcttgtctgagacctctggcctggggaagaccacagacatgtcgaggataagcttggaacctggcacaagtttacctcccaatttgagcagtacagcaggtgaggcgttatccacttatgaagcctccagagatacaaaggcaattcatcattctgcagacacagcagtgacgaatatggaggcaaccagttctgaatattctcctatcccaggccatacaaagccatccaaagccacatctccattggttacctcccacatcatgggggacatcacttcttccacatcagtatttggctcctccgagaccacagagattgagacagtgtcctctgtgaaccagggacttcaggagagaagcacatcccaggtggccagctctgctacagagacaagcactgtcattacccatgtgtctagtggtgatgctactactcatgtcaccaagacacaagccactttctctagcggaacatccatctcaagccctcatcagtttataacttctaccaacacatttacagatgtgagcaccaacccctccacctctctgataatgacagaatcttcaggagtgaccatcaccacccaaacaggtcctactggagctgcaacacagggtccatatctcttggacacatcaaccatgccttacttgacagagactccattagctgtgactccagattttatgcaatcagagaagaccactctcataagcaaaggtcccaaggatgtgacctggacaagccctccctctgtggcagaaaccagctatccctcttccctgacacctttcttggtcacaaccatacctcctgccacttccacgttacaagggcaacatacatcctctcctgtttctgcgacttcagttcttacctctggactggtgaagaccacagatatgttgaacacaagcatggaacctgtgaccaattcacctcaaaatttgaacaatccatcaaatgagatactggccactttggcagccaccacagatatagagactattcatccttccataaacaaagcagtgaccaatatggggactgccagttcagcacatgtactgcattccactctcccagtcagctcagaaccatctacagccacatctccaatggttcctgcctccagcatgggggacgctcttgcttctatatcaatacctggttctgagaccacagacattgagggagagccaacatcctccctgactgctggacgaaaagagaacagcaccctccaggagatgaactcaactacagagtcaaacatcatcctctccaatgtgtctgtgggggctattactgaagccacaaaaatggaagtcccctcttttgatgcaacattcataccaactcctgctcagtcaacaaagttcccagatattttctcagtagccagcagtagactttcaaactctcctcccatgacaatatctacccacatgaccaccacccagacagggtcttctggagctacatcaaagattccacttgccttagacacatcaaccttggaaacctcagcagggactccatcagtggtgactgaggggtttgcccactcaaaaataaccactgcaatgaacaatgatgtcaaggacgtgtcacagacaaaccctccctttcaggatgaagccagctctccctcttctcaagcacctgtccttgtcacaaccttaccttcttctgttgctttcacaccgcaatggcacagtacctcctctcctgtttctatgtcctcagttcttacttcttcactggtaaagaccgcaggcaaggtggatacaagcttagaaacagtgaccagttcacctcaaagtatgagcaacactttggatgacatatcggtcacttcagcagccaccacagatatagagacaacgcatccttccataaacacagtagttaccaatgtggggaccaccggttcagcatttgaatcacattctactgtctcagcttacccagagccatctaaagtcacatctccaaatgttaccacctccaccatggaagacaccacaatttcccgatcaatacctaaatcctctaagactacaagaactgagactgagacaacttcctccctgactcctaaactgagggagaccagcatctcccaggagatcacctcgtccacagagacaagcactgttccttacaaagagctcactggtgccactaccgaggtatccaggacagatgtcacttcctctagcagtacatccttccctggccctgatcagtccacagtgtcactagacatctccacagaaaccaacaccaggctgtctacctccccaataatgacagaatctgcagaaataaccatcaccacccaaacaggtcctcatggggctacatcacaggatacttttaccatggacccatcaaatacaaccccccaggcagggatccactcagctatgactcatggattttcacaattggatgtgaccactcttatgagcagaattccacaggatgtatcatggacaagtcctccctctgtggataaaaccagctccccctcttcctttctgtcctcacctgcaatgaccacaccttccctgatttcttctaccttaccagaggataagctctcctctcctatgacttcacttctcacctctggcctagtgaagattacagacatattacgtacacgcttggaacctgtgaccagctcacttccaaatttcagcagcacctcagataagatactggccacttctaaagacagtaaagacacaaaggaaatttttccttctataaacacagaagagaccaatgtgaaagccaacaactctggacatgaatcccattcccctgcactggctgactcagagacacccaaagccacaactcaaatggttatcaccaccactgtgggagatccagctccttccacatcaatgccagtgcatggttcctctgagactacaaacattaagagagagccaacatatttcttgactcctagactgagagagaccagtacctctcaggagtccagctttcccacggacacaagttttctactttccaaagtccccactggtactattactgaggtctccagtacaggggtcaactcttctagcaaaatttccaccccagaccatgataagtccacagtgccacctgacaccttcacaggagagatccccagggtcttcacctcctctattaagacaaaatctgcagaaatgacgatcaccacccaagcaagtcctcctgagtctgcatcgcacagtacccttcccttggacacatcaaccacactttcccagggagggactcattcaactgtgactcagggattcccatactcagaggtgaccactctcatgggcatgggtcctgggaatgtgtcatggatgacaactccccctgtggaagaaaccagctctgtgtcttccctgatgtcttcacctgccatgacatccccttctcctgtttcctccacatcaccacagagcatcccctcctctcctcttcctgtgactgcacttcctacttctgttctggtgacaaccacagatgtgttgggcacaacaagcccagagtctgtaaccagttcacctccaaatttgagcagcatcactcatgagagaccggccacttacaaagacactgcacacacagaagccgccatgcatcattccacaaacaccgcagtgaccaatgtagggacttccgggtctggacataaatcacaatcctctgtcctagctgactcagagacatcgaaagccacacctctgatgagtaccacctccaccctgggggacacaagtgtttccacatcaactcctaatatctctcagactaaccaaattcaaacagagccaacagcatccctgagccctagactgagggagagcagcacgtctgagaagaccagctcaacaacagagacaaatactgccttttcttatgtgcccacaggtgctattactcaggcctccagaacagaaatctcctctagcagaacatccatctcagaccttgatcggcccacaatagcacccgacatctccacaggaatgatcaccaggctcttcacctcccccatcatgacaaaatctgcagaaatgaccgtcaccactcaaacaactactcctggggctacatcacagggtatccttccttgggacacatcaaccacacttttccagggagggactcattcaaccgtgtctcagggattcccacactcagagataaccactcttcggagcagaacccctggagatgtgtcatggatgacaactccccctgtggaagaaaccagctctgggttttccctgatgtcaccttccatgacatccccttctcctgtttcctccacatcaccagagagcatcccctcctctcctctccctgtgactgcacttcttacttctgttctggtgacaaccaccaatgtattgggcacaacaagcccagagaccgtaacgagttcacctccaaatttaagcagccccacacaggagagactgaccacttacaaagacactgcgcacacagaagccatgcatgcttccatgcatacaaacactgcagtggccaacgtcgggacctccatttctggacatgaatcacaatcttctgtcccagctgattcacacacatccaaagccacatctccaatgggtatcaccttcgccatgggggatacaagtgtttctacatcaactcctgccttctttgagactagaattcagactgaatcaacatcctctttgattcctggattaagggacaccaggacgtctgaggagatcaacactgtgacagagaccagcactgtcctttcagaagtgcccactactactactactgaggtctccaggacagaagttatcacttccagcagaacaaccatctcagggcctgatcattccaaaatgtcaccctacatctccacagaaaccatcaccaggctctccacttttccttttgtaacaggatccacagaaatggccatcaccaaccaaacaggtcctatagggactatctcacaggctacccttaccctggacacatcaagcacagcttcctgggaagggactcactcacctgtgactcagagatttccacactcagaggagaccactactatgagcagaagtactaagggcgtgtcatggcaaagccctccctctgtggaagaaaccagttctccttcttccccagtgcctttacctgcaataacctcacattcatctctttattccgcagtatcaggaagtagccccacttctgctctccctgtgacttcccttctcacctctggcaggaggaagaccatagacatgttggacacacactcagaacttgtgaccagctccttaccaagtgcaagtagcttctcaggtgagatactcacttctgaagcctccacaaatacagagacaattcacttttcagagaacacagcagaaaccaatatggggaccaccaattctatgcataaactacattcctctgtctcaatccactcccagccatccggacacacacctccaaaggttactggatctatgatggaggacgctattgtttccacatcaacacctggttctcctgagactaaaaatgttgacagagactcaacatcccctctgactcctgaactgaaagaggacagcaccgccctggtgatgaactcaactacagagtcaaacactgttttctccagtgtgtccctggatgctgctactgaggtctccagggcagaagtcacctactatgatcctacattcatgccagcttctgctcagtcaacaaagtccccagacatttcacctgaagccagcagcagtcattctaactctcctcccttgacaatatctacacacaagaccatcgccacacaaacaggtccttctggggtgacatctcttggccaactgaccctggacacatcaaccatagccacctcagcaggaactccatcagccagaactcaggattttgtagattcagaaacaaccagtgtcatgaacaatgatctcaatgatgtgttgaagacaagccctttctctgcagaagaagccaactctctctcttctcaggcacctctccttgtgacaacctcaccttctcctgtaacttccacattgcaagagcacagtacctcctctcttgtttctgtgacctcagtacccacccctacactggcgaagatcacagacatggacacaaacttagaacctgtgactcgttcacctcaaaatttaaggaacaccttggccacttcagaagccaccacagatacacacacaatgcatccttctataaacacagcaatggccaatgtggggaccaccagttcaccaaatgaattctattttactgtctcacctgactcagacccatataaagccacatccgcagtagttatcacttccacctcgggggactcaatagtttccacatcaatgcctagatcctctgcgatgaaaaagattgagtctgagacaactttctccctgatatttagactgagggagactagcacctcccagaaaattggctcatcctcagacacaagcacggtctttgacaaagcattcactgctgctactactgaggtctccagaacagaactcacctcctctagcagaacatccatccaaggcactgaaaagcccacaatgtcaccggacacctccacaagatctgtcaccatgctttctacttttgctggcctgacaaaatccgaagaaaggaccattgccacccaaacaggtcctcatagggcgacatcacagggtacccttacctgggacacatcaatcacaacctcacaggcagggacccactcagctatgactcatggattttcacaattagatttgtccactcttacgagtagagttcctgagtacatatcagggacaagcccaccctctgtggaaaaaaccagctcttcctcttcccttctgtctttaccagcaataacctcaccgtcccctgtacctactacattaccagaaagtaggccgtcttctcctgttcatctgacttcactccccacctctggcctagtgaagaccacagatatgctggcatctgtggccagtttacctccaaacttgggcagcacctcacataagataccgactacttcagaagacattaaagatacagagaaaatgtatccttccacaaacatagcagtaaccaatgtggggaccaccacttctgaaaaggaatcttattcgtctgtcccagcctactcagaaccacccaaagtcacctctccaatggttacctctttcaacataagggacaccattgtttccacatccatgcctggctcctctgagattacaaggattgagatggagtcaacattctccgtggctcatgggctgaagggaaccagcacctcccaggaccccatcgtatccacagagaaaagtgctgtccttcacaagttgaccactggtgctactgagacctctaggacagaagttgcctcttctagaagaacatccattccaggccctgatcattccacagagtcaccagacatctccactgaagtgatccccagcctgcctatctcccttggcattacagaatcttcaaatatgaccatcatcactcgaacaggtcctcctcttggctctacatcacagggcacatttaccttggacacaccaactacatcctccagggcaggaacacactcgatggcgactcaggaatttccacactcagaaatgaccactgtcatgaacaaggaccctgagattctatcatggacaatccctccttctatagagaaaaccagcttctcctcttccctgatgccttcaccagccatgacttcacctcctgtttcctcaacattaccaaagaccattcacaccactccttctcctatgacctcactgctcacccctagcctagtgatgaccacagacacattgggcacaagcccagaacctacaaccagttcacctccaaatttgagcagtacctcacatgtgatactgacaacagatgaagacaccacagctatagaagccatgcatccttccacaagcacagcagcgactaatgtggaaaccacctgttctggacatgggtcacaatcctctgtcctaactgactcagaaaaaaccaaggccacagctccaatggataccacctccaccatggggcatacaactgtttccacatcaatgtctgtttcctctgagactacaaaaattaagagagagtcaacatattccttgactcctggactgagagagaccagcatttcccaaaatgccagcttttccactgacacaagtattgttctttcagaagtccccactggtactactgctgaggtctccaggacagaagtcacctcctctggtagaacatccatccctggcccttctcagtccacagttttgccagaaatatccacaagaacaatgacaaggctctttgcctcgcccaccatgacagaatcagcagaaatgaccatccccactcaaacaggtccttctgggtctacctcacaggatacccttaccttggacacatccaccacaaagtcccaggcaaagactcattcaactttgactcagagatttccacactcagagatgaccactctcatgagcagaggtcctggagatatgtcatggcaaagctctccctctctggaaaatcccagctctctcccttccctgctgtctttacctgccacaacctcacctcctcccatttcctccacattaccagtgactatctcctcctctcctcttcctgtgacttcacttctcacctctagcccggtaacgaccacagacatgttacacacaagcccagaacttgtaaccagttcacctccaaagctgagccacacttcagatgagagactgaccactggcaaggacaccacaaatacagaagctgtgcatccttccacaaacacagcagcgtccaatgtggagattcccagctttggacatgaatccccttcctctgccttagctgactcagagacatccaaagccacatcaccaatgtttattacctccacccaggaggatacaactgttgccatatcaacccctcacttcttggagactagcagaattcagaaagagtcaatttcctccctgagccctaaattgagggagacaggcagttctgtggagacaagctcagccatagagacaagtgctgtcctttctgaagtgtccattggtgctactactgagatctccaggacagaagtcacctcctctagcagaacatccatctctggttctgctgagtccacaatgttgccagaaatatccaccacaagaaaaatcattaagttccctacttcccccatcctggcagaatcatcagaaatgaccatcaagacccaaacaagtcctcctgggtctacatcagagagtacctttacattagacacatcaaccactccctccttggtaataacccattcgactatgactcagagattgccacactcagagataaccactcttgtgagtagaggtgctggggatgtgccacggcccagctctctccctgtggaagaaacaagccctccatcttcccagctgtctttatctgccatgatctcaccttctcctgtttcttccacattaccagcaagtagccactcctcttctgcttctgtgacttcacctctcacaccaggccaagtgaagactactgaggtgttggacgcaagtgcagaacctgaaaccagttcacctccaagtttgagcagcacctcagttgaaatactggccacctctgaagtcaccacagatacggagaaaattcatcctttcccaaacacggcagtaaccaaagttggaacttccagttctggacatgaatccccttcctctgtcctacctgactcagagacaaccaaagccacatcggcaatgggtaccatctccattatgggggatacaagtgtttctacattaactcctgccttatctaacactaggaaaattcagtcagagccagcttcctcactgaccaccagattgagggagaccagcacctctgaagagaccagcttagccacagaagcaaacactgttctttctaaagtgtccactggtgctactactgaggtctccaggacagaagccatctcctttagcagaacatccatgtcaggccctgagcagtccacaatgtcacaagacatctccataggaaccatccccaggatttctgcctcctctgtcctgacagaatctgcaaaaatgaccatcacaacccaaacaggtccttcggagtctacactagaaagtacccttaatttgaacacagcaaccacaccctcttgggtggaaacccactctatagtaattcagggatttccacacccagagatgaccacttccatgggcagaggtcctggaggtgtgtcatggcctagccctccctttgtgaaagaaaccagccctccatcctccccgctgtctttacctgccgtgacctcacctcatcctgtttccaccacattcctagcacatatccccccctctccccttcctgtgacttcacttctcacctctggcccggcgacaaccacagatatcttgggtacaagcacagaacctggaaccagttcatcttcaagtttgagcaccacctcccatgagagactgaccacttacaaagacactgcacatacagaagccgtgcatccttccacaaacacaggagggaccaatgtggcaaccaccagctctggatataaatcacagtcctctgtcctagctgactcatctccaatgtgtaccacctccaccatgggggatacaagtgttctcacatcaactcctgccttccttgagactaggaggattcagacagagctagcttcctccctgacccctggattgagggagtccagtggctctgaagggaccagctcaggcaccaagatgagcactgtcctctctaaagtgcccactggtgctactactgagatctccaaggaagacgtcacctccatcccaggtcccgctcaatccacaatatcaccagacatctccacaagaaccgtcagctggttctctacatcccctgtcatgacagaatcagcagaaataaccatgaacacccatacaagtcctttaggggccacaacacaaggcaccagtactttggccacgtcaagcacaacctctttgacaatgacacactcaactatatctcaaggattttcacactcacagatgagcactcttatgaggaggggtcctgaggatgtatcatggatgagccctccccttctggaaaaaactagaccttccttttctctgatgtcttcaccagccacaacttcaccttctcctgtttcctccacattaccagagagcatctcttcctctcctcttcctgtgacttcactcctcacgtctggcttggcaaaaactacagatatgttgcacaaaagctcagaacctgtaaccaactcacctgcaaatttgagcagcacctcagttgaaatactggccacctctgaagtcaccacagatacagagaaaactcatccttcttcaaacagaacagtgaccgatgtggggacctccagttctggacatgaatccacttcctttgtcctagctgactcacagacatccaaagtcacatctccaatggttattacctccaccatggaggatacgagtgtctccacatcaactcctggcttttttgagactagcagaattcagacagaaccaacatcctccctgacccttggactgagaaagaccagcagctctgaggggaccagcttagccacagagatgagcactgtcctttctggagtgcccactggtgccactgctgaagtctccaggacagaagtcacctcctctagcagaacatccatctcaggctttgctcagctcacagtgtcaccagagacttccacagaaaccatcaccagactccctacctccagcataatgacagaatcagcagaaatgatgatcaagacacaaacagatcctcctgggtctacaccagagagtactcatactgtggacatatcaacaacacccaactgggtagaaacccactcgactgtgactcagagattttcacactcagagatgaccactcttgtgagcagaagccctggtgatatgttatggcctagtcaatcctctgtggaagaaaccagctctgcctcttccctgctgtctctgcctgccacgacctcaccttctcctgtttcctctacattagtagaggatttcccttccgcttctcttcctgtgacttctcttctcacccctggcctggtgataaccacagacaggatgggcataagcagagaacctggaaccagttccacttcaaatttgagcagcacctcccatgagagactgaccactttggaagacactgtagatacagaagacatgcagccttccacacacacagcagtgaccaacgtgaggacctccatttctggacatgaatcacaatcttctgtcctatctgactcagagacacccaaagccacatctccaatgggtaccacctacaccatgggggaaacgagtgtttccatatccacttctgacttctttgagaccagcagaattcagatagaaccaacatcctccctgacttctggattgagggagaccagcagctctgagaggatcagctcagccacagagggaagcactgtcctttctgaagtgcccagtggtgctaccactgaggtctccaggacagaagtgatatcctctaggggaacatccatgtcagggcctgatcagttcaccatatcaccagacatctctactgaagcgatcaccaggctttctacttcccccattatgacagaatcagcagaaagtgccatcactattgagacaggttctcctggggctacatcagagggtaccctcaccttggacacctcaacaacaaccttttggtcagggacccactcaactgcatctccaggattttcacactcagagatgaccactcttatgagtagaactcctggagatgtgccatggccgagccttccctctgtggaagaagccagctctgtctcttcctcactgtcttcacctgccatgacctcaacttcttttttctccgcattaccagagagcatctcctcctctcctcatcctgtgactgcacttctcacccttggcccagtgaagaccacagacatgttgcgcacaagctcagaacctgaaaccagttcacctccaaatttgagcagcacctcagctgaaatattagccacgtctgaagtcaccaaagatagagagaaaattcatccctcctcaaacacacctgtagtcaatgtagggactgtgatttataaacatctatccccttcctctgttttggctgacttagtgacaacaaaacccacatctccaatggctaccacctccactctggggaatacaagtgtttccacatcaactcctgccttcccagaaactatgatgacacagccaacttcctccctgacttctggattaagggagatcagtacctctcaagagaccagctcagcaacagagagaagtgcttctctttctggaatgcccactggtgctactactaaggtctccagaacagaagccctctccttaggcagaacatccaccccaggtcctgctcaatccacaatatcaccagaaatctccacggaaaccatcactagaatttctactcccctcaccacgacaggatcagcagaaatgaccatcacccccaaaacaggtcattctggggcatcctcacaaggtacctttaccttggacacatcaagcagagcctcctggccaggaactcactcagctgcaactcacagatctccacactcagggatgaccactcctatgagcagaggtcctgaggatgtgtcatggccaagccgcccatcagtggaaaaaactagccctccatcttccctggtgtctttatctgcagtaacctcaccttcgccactttattccacaccatctgagagtagccactcatctcctctccgggtgacttctcttttcacccctgtcatgatgaagaccacagacatgttggacacaagcttggaacctgtgaccacttcacctcccagtatgaatatcacctcagatgagagtctggccacttctaaagccaccatggagacagaggcaattcagctttcagaaaacacagctgtgactcagatgggcaccatcagcgctagacaagaattctattcctcttatccaggcctcccagagccatccaaagtgacatctccagtggtcacctcttccaccataaaagacattgtttctacaaccatacctgcttcctctgagataacaagaattgagatggagtcaacatccaccctgacccccacaccaagggagaccagcacctcccaggagatccactcagccacaaagccaagcactgttccttacaaggcactcactagtgccacgattgaggactccatgacacaagtcatgtcctctagcagaggacctagccctgatcagtccacaatgtcacaagacatatccagtgaagtgatcaccaggctctctacctcccccatcaaggcagaatctacagaaatgaccattaccacccaaacaggttctcctggggctacatcaaggggtacccttaccttggacacttcaacaacttttatgtcagggacccactcaactgcatctcaaggattttcacactcacagatgaccgctcttatgagtagaactcctggagatgtgccatggctaagccatccctctgtggaagaagccagctctgcctctttctcactgtcttcacctgtcatgacctcatcttctcccgtttcttccacattaccagacagcatccactcttcttcgcttcctgtgacatcacttctcacctcagggctggtgaagaccacagagctgttgggcacaagctcagaacctgaaaccagttcacccccaaatttgagcagcacctcagctgaaatactggccaccactgaagtcactacagatacagagaaactggagatgaccaatgtggtaacctcaggttatacacatgaatctccttcctctgtcctagctgactcagtgacaacaaaggccacatcttcaatgggtatcacctaccccacaggagatacaaatgttctcacatcaacccctgccttctctgacaccagtaggattcaaacaaagtcaaagctctcactgactcctgggttgatggagaccagcatctctgaagagaccagctctgccacagaaaaaagcactgtcctttctagtgtgcccactggtgctactactgaggtctccaggacagaagccatctcttctagcagaacatccatcccaggccctgctcaatccacaatgtcatcagacacctccatggaaaccatcactagaatttctacccccctcacaaggaaagaatcaacagacatggccatcacccccaaaacaggtccttctggggctacctcgcagggtacctttaccttggactcatcaagcacagcctcctggccaggaactcactcagctacaactcagagatttccacagtcagtggtgacaactcctatgagcagaggtcctgaggatgtgtcatggccaagcccgctgtctgtggaaaaaaacagccctccatcttccctggtatcttcatcttcagtaacctcaccttcgccactttattccacaccatctgggagtagccactcctctcctgtccctgtcacttctcttttcacctctatcatgatgaaggccacagacatgttggatgcaagtttggaacctgagaccacttcagctcccaatatgaatatcacctcagatgagagtctggccacttctaaagccaccacggagacagaggcaattcacgtttttgaaaatacagcagcgtcccatgtggaaaccaccagtgctacagaggaactctattcctcttccccaggcttctcagagccaacaaaagtgatatctccagtggtcacctcttcctctataagagacaacatggtttccacaacaatgcctggctcctctggcattacaaggattgagatagagtcaatgtcatctctgacccctggactgagggagaccagaacctcccaggacatcacctcatccacagagacaagcactgtcctttacaagatgtcctctggtgccactcctgaggtctccaggacagaagttatgccctctagcagaacatccattcctggccctgctcagtccacaatgtcactagacatctccgatgaagttgtcaccaggctgtctacctctcccatcatgacagaatctgcagaaataaccatcaccacccaaacaggttattctctggctacatcccaggttacccttcccttgggcacctcaatgacctttttgtcagggacccactcaactatgtctcaaggactttcacactcagagatgaccaatcttatgagcaggggtcctgaaagtctgtcatggacgagccctcgctttgtggaaacaactagatcttcctcttctctgacatcattacctctcacgacctcactttctcctgtgtcctccacattactagacagtagcccctcctctcctcttcctgtgacttcacttatcctcccaggcctggtgaagactacagaagtgttggatacaagctcagagcctaaaaccagttcatctccaaatttgagcagcacctcagttgaaataccggccacctctgaaatcatgacagatacagagaaaattcatccttcctcaaacacagcggtggccaaagtgaggacctccagttctgttcatgaatctcattcctctgtcctagctgactcagaaacaaccataaccataccttcaatgggtatcacctccgctgtggacgataccactgttttcacatcaaatcctgccttctctgagactaggaggattccgacagagccaacattctcattgactcctggattcagggagactagcacctctgaagagaccacctcaatcacagaaacaagtgcagtcctttatggagtgcccactagtgctactactgaagtctccatgacagaaatcatgtcctctaatagaacacacatccctgactctgatcagtccacgatgtctccagacatcatcactgaagtgatcaccaggctctcttcctcatccatgatgtcagaatcaacacaaatgaccatcaccacccaaaaaagttctcctggggctacagcacagagtactcttaccttggccacaacaacagcccccttggcaaggacccactcaactgttcctcctagatttttacactcagagatgacaactcttatgagtaggagtcctgaaaatccatcatggaagagctctccctttgtggaaaaaactagctcttcatcttctctgttgtccttacctgtcacgacctcaccttctgtttcttccacattaccgcagagtatcccttcctcctctttttctgtgacttcactcctcaccccaggcatggtgaagactacagacacaagcacagaacctggaaccagtttatctccaaatctgagtggcacctcagttgaaatactggctgcctctgaagtcaccacagatacagagaaaattcatccttcttcaagcatggcagtgaccaatgtgggaaccaccagttctggacatgaactatattcctctgtttcaatccactcggagccatccaaggctacatacccagtgggtactccctcttccatggctgaaacctctatttccacatcaatgcctgctaattttgagaccacaggatttgaggctgagccattttctcatttgacttctggatttaggaagacaaacatgtccctggacaccagctcagtcacaccaacaaatacaccttcttctcctgggtccactcaccttttacagagttccaagactgatttcacctcttctgcaaaaacatcatccccagactggcctccagcctcacagtatactgaaattccagtggacataatcaccccctttaatgcttctccatctattacggagtccactgggataacctccttcccagaatccaggtttactatgtctgtaacagaaagtactcatcatctgagtacagatttgctgccttcagctgagactatttccactggcacagtgatgccttctctatcagaggccatgacttcatttgccaccactggagttccacgagccatctcaggttcaggtagtccattctctaggacagagtcaggccctggggatgctactctgtccaccattgcagagagcctgccttcatccactcctgtgccattctcctcttcaaccttcactaccactgattcttcaaccatcccagccctccatgagataacttcctcttcagctaccccatatagagtggacaccagtcttgggacagagagcagcactactgaaggacgcttggttatggtcagtactttggacacttcaagccaaccaggcaggacatcttcaacacccattttggataccagaatgacagagagcgttgagctgggaacagtgacaagtgcttatcaagttccttcactctcaacacggttgacaagaactgatggcattatggaacacatcacaaaaatacccaatgaagcagcacacagaggtaccataagaccagtcaaaggccctcagacatccacttcgcctgccagtcctaaaggactacacacaggagggacaaaaagaatggagaccaccaccacagctttgaagaccaccaccacagctttgaagaccacttccagagccaccttgaccaccagtgtctatactcccactttgggaacactgactcccctcaatgcatcaaggcaaatggccagcacaatcctcacagaaatgatgatcacaaccccatatgttttccctgatgttccagaaacgacatcctcattggctaccagcctgggagcagaaaccagcacagctcttcccaggacaaccccatctgttctcaatagagaatcagagaccacagcctcactggtctctcgttctggggcagagagaagtccggttattcaaactctagatgtttcttctagtgagccagatacaacagcttcatgggttatccatcctgcagagaccatcccaactgtttccaagacaacccccaattttttccacagtgaattagacactgtatcttccacagccaccagtcatggggcagacgtcagctcagccattccaacaaatatctcacctagtgaactagatgcactgaccccactggtcactatttcggggacagatactagtacaacattcccaacactgactaagtccccacatgaaacagagacaagaaccacatggctcactcatcctgcagagaccagctcaactattcccagaacaatccccaatttttctcatcatgaatcagatgccacaccttcaatagccaccagtcctggggcagaaaccagttcagctattccaattatgactgtctcacctggtgcagaagatctggtgacctcacaggtcactagttctgggacagacagaaatatgactattccaactttgactctttctcctggtgaaccaaagacgatagcctcattagtcacccatcctgaagcacagacaagttcggccattccaacttcaactatctcgcctgctgtatcacggttggtgacctcaatggtcaccagtttggcggcaaagacaagtacaactaatcgagctctgacaaactcccctggtgaaccagctacaacagtttcattggtcacgcatcctgcacagaccagcccaacagttccctggacaacttccatttttttccatagtaaatcagacaccacaccttcaatgaccaccagtcatggggcagaatccagttcagctgttccaactccaactgtttcaactgaggtaccaggagtagtgacccctttggtcaccagttctagggcagtgatcagtacaactattccaattctgactctttctcctggtgaaccagagaccacaccttcaatggccaccagtcatggggaagaagccagttctgctattccaactccaactgtttcacctggggtaccaggagtggtgacctctctggtcactagttctagggcagtgactagtacaactattccaattctgactttttctcttggtgaaccagagaccacaccttcaatggccaccagtcatgggacagaagctggctcagctgttccaactgttttacctgaggtaccaggaatggtgacctctctggttgctagttctagggcagtaaccagtacaactcttccaactctgactctttctcctggtgaaccagagaccacaccttcaatggccaccagtcatggggcagaagccagctcaactgttccaactgtttcacctgaggtaccaggagtggtgacctctctggtcactagttctagtggagtaaacagtacaagtattccaactctgattctttctcctggtgaactagaaaccacaccttcaatggccaccagtcatggggcagaagccagctcagctgttccaactccaactgtttcacctggggtatcaggagtggtgacccctctggtcactagttccagggcagtgaccagtacaactattccaattctaactctttcttctagtgagccagagaccacaccttcaatggccaccagtcatggggtagaagccagctcagctgttctaactgtttcacctgaggtaccaggaatggtgacctctctggtcactagttctagagcagtaaccagtacaactattccaactctgactatttcttctgatgaaccagagaccacaacttcattggtcacccattctgaggcaaagatgatttcagccattccaactttagctgtctcccctactgtacaagggctggtgacttcactggtcactagttctgggtcagagaccagtgcgttttcaaatctaactgttgcctcaagtcaaccagagaccatagactcatgggtcgctcatcctgggacagaagcaagttctgttgttccaactttgactgtctccactggtgagccgtttacaaatatctcattggtcacccatcctgcagagagtagctcaactcttcccaggacaacctcaaggttttcccacagtgaattagacactatgccttctacagtcaccagtcctgaggcagaatccagctcagccatttcaactactatttcacctggtataccaggtgtgctgacatcactggtcactagctctgggagagacatcagtgcaacttttccaacagtgcctgagtccccacatgaatcagaggcaacagcctcatgggttactcatcctgcagtcaccagcacaacagttcccaggacaacccctaattattctcatagtgaaccagacaccacaccatcaatagccaccagtcctggggcagaagccacttcagattttccaacaataactgtctcacctgatgtaccagatatggtaacctcacaggtcactagttctgggacagacaccagtataactattccaactctgactctttcttctggtgagccagagaccacaacctcatttatcacctattctgagacacacacaagttcagccattccaactctccctgtctcccctggtgcatcaaagatgctgacctcactggtcatcagttctgggacagacagcactacaactttcccaacactgacggagaccccatatgaaccagagacaacagccatacagctcattcatcctgcagagaccaacacaatggttcccaagacaactcccaagttttcccatagtaagtcagacaccacactcccagtagccatcaccagtcctgggccagaagccagttcagctgtttcaacgacaactatctcacctgatatgtcagatctggtgacctcactggtccctagttctgggacagacaccagtacaaccttcccaacattgagtgagaccccatatgaaccagagactacagtcacgtggctcactcatcctgcagaaaccagcacaacggtttctgggacaattcccaacttttcccataggggatcagacactgcaccctcaatggtcaccagtcctggagtagacacgaggtcaggtgttccaactacaaccatcccacccagtataccaggggtagtgacctcacaggtcactagttctgcaacagacactagtacagctattccaactttgactccttctcctggtgaaccagagaccacagcctcatcagctacccatcctgggacacagactggcttcactgttccaattcggactgttccctctagtgagccagatacaatggcttcctgggtcactcatcctccacagaccagcacacctgtttccagaacaacctccagtttttcccatagtagtccagatgccacacctgtaatggccaccagtcctaggacagaagccagttcagctgtactgacaacaatctcacctggtgcaccagagatggtgacttcacagatcactagttctggggcagcaaccagtacaactgttccaactttgactcattctcctggtatgccagagaccacagccttattgagcacccatcccagaacagggacaagtaaaacatttcctgcttcaactgtgtttcctcaagtatcagagaccacagcctcactcaccattagacctggtgcagagactagcacagctctcccaactcagacaacatcctctctcttcaccctacttgtaactggaaccagcagagttgatctaagtccaactgcttcacctggtgtttctgcaaaaacagccccactttccacccatccagggacagagaccagcacaatgattccaacttcaactctttcccttggtttactagagactacaggcttactggccaccagctcttcagcagagaccagcacgagtactctaactctgactgtttcccctgctgtctctgggctttccagtgcctctataacaactgataagccccaaactgtgacctcctggaacacagaaacctcaccatctgtaacttcagttggacccccagaattttccaggactgtcacaggcaccactatgaccttgataccatcagagatgccaacaccacctaaaaccagtcatggagaaggagtgagtccaaccactatcttgagaactacaatggttgaagccactaatttagctaccacaggttccagtcccactgtggccaagacaacaaccaccttcaatacactggctggaagcctctttactcctctgaccacacctgggatgtccaccttggcctctgagagtgtgacctcaagaacaagttataaccatcggtcctggatctccaccaccagcagttataaccgtcggtactggacccctgccaccagcactccagtgacttctacattctccccagggatttccacatcctccatccccagctccacagcagccacagtcccattcatggtgccattcaccctcaacttcaccatcaccaacctgcagtacgaggaggacatgcggcaccctggttccaggaagttcaacgccacagagagagaactgcagggtctgctcaaacccttgttcaggaatagcagtctggaatacctctattcaggctgcagactagcctcactcaggccagagaaggatagctcagccatggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatggtttcacccatcgaagctctatgcccaccaccagcactcctgggacctccacagtggatgtgggaacctcagggactccatcctccagccccagccccacggctgctggccctctcctgatgccgttcaccctcaacttcaccatcaccaacctgcagtacgaggaggacatgcgtcgcactggctccaggaagttcaacaccatggagagtgtcctgcagggtctgctcaagcccttgttcaagaacaccagtgttggccctctgtactctggctgcagattgaccttgctcaggcccgagaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatggtttcacccatcagagctctgtgtccaccaccagcactcctgggacctccacagtggatctcagaacctcagggactccatcctccctctccagccccacaattatggctgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtctgagaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatggtttcacccatcggacctctgtgcccaccaccagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagccactggagtggatgccatctgcacccaccgtcttgaccccaaaagccctggactggacagagagcagctatactgggagctgagccagctgaccaatggcatcaaagagctgggcccctacaccctggacaggaacagtctctatgtcaatggtttcacccattggatccctgtgcccaccagcagcactcctgggacctccacagtggaccttgggtcagggactccatcctccctccccagccccacagctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggtccctacaccctggacagaaacagtctctatgtcaatggtttcacccatcagacctctgcgcccaacaccagcactcctgggacctccacagtggaccttgggacctcagggactccatcctccctccccagccctacatcngctggccctctcctggtnccnttcaccctcaacttcaccatcaccaacctgcagtacgaggaggacatgcggcacccnggntccaggaagttcaacaccacngagagggtnctgcagggtctgctnaagcccctnttcaagagcaccagtgttggccctctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggtccctacaccctggacagaaacagtctctatgtcaatggtttcacccatcagacctctgcgcccaacaccagcactcctgggacctccacagtggaccttgggacctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcaacagagagcagctgtactgggagctgagccagctgacccatggcatcaaagagctgggcccctacaccctggacaggaacagtctctatgtcaatggtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccagatgaccaatggcatcaaagagctgggcccctacaccctggaccggaacagtctctacgtcaatggtttcacccatcggagctctgggctcaccaccagcactccttggacttccacagttgaccttggaacctcagggactccatcccccgtccccagccccacaactgctggccctctcctggtgccattcaccctcaacttcaccatcaccaacctgcagtatgaggaggacatgcatcgccctggatctaggaagttcaacaccacagagagggtcctgcagggtctgcttagtcccattttcaagaactccagtgttggccctctgtactctggctgcagactgacctctctcaggcccgagaaggatggggcagcaactggaatggatgctgtctgcctctaccaccctaatcccaaaagacctggactggacagagagcagctgtactgggagctaagccagctgacccacaacatcactgagctgggcccctacagcctggacagggacagtctctatgtcaatggtttcacccatcagaactctgtgcccaccaccagtactcctgggacctccacagtgtactgggcaaccactgggactccatcctccttccccggccacacagagcctggccctctcctgataccattcactttcaactttaccatcaccaacctgcattatgaggaaaacatgcaacaccctggttccaggaagttcaacaccacggagagggttctgcagggtctgctcaagcccttgttcaagaacaccagtgttggccctctgtactctggctgcagactgacctctctcaggcccgagaaggatggggcagcaactggaatggatgctgtctgcctctaccaccctaatcccaaaagacctgggctggacagagagcagctgtactgggagctaagccagctgacccacaacatcactgagctgggcccctacagcctggacagggacagtctctatgtcaatggtttcacccatcagaactctgtgcccaccaccagtactcctgggacctccacagtgtactgggcaaccactgggactccatcctccttccccggccacacagagcctggccctctcctgataccattcactttcaactttaccatcaccaacctgcattatgaggaaaacatgcaacaccctggttccaggaagttcaacaccacggagagggttctgcagggtctgctcaagcccttgttcaagaacaccagtgttggccctctgtactctggctgcagactgaccttgctcagacctgagaagcatgaggcagccactggagtggacaccatctgtacccaccgcgttgatcccatcggacctggactggacagggagcggctatactgggagctgagccagctgaccaacagcattaccgaactgggaccctacaccctggacagggacagtctctatgtcaatggcttcaaccctcggagctctgtgccaaccaccagcactcctgggacctccacagtgcacctggcaacctctgggactccatcctccctgcctggccacacagcccctgtccctctcttgataccattcaccctcaactttaccatcaccaacctgcattatgaggaaaacatgcaacaccctggttccaggaagttcaacaccacggagagggttctgcagggtctgctcaagcccttgttcaagaacaccagtgttggccctctgtactctggctgcagactgaccttgctcagacctgagaagcatgaggcagccactggagtggacaccatctgtacccaccgcgttgatcccatcggacctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccactggaatggatgccatctgcagccaccgtcttgaccccaaaagccctggactcgacagagagcagctgtactgggagctgagccagctgacccatggcatcaaagagctgggcccctacaccctggacaggaacagtctctatgtcaatggtttcacccatcggagctctgtggcccccaccagcactcctgggacctccacagtggaccttgggacctcagggactccatcctccctccccagccccacaacagctgttcctctcctggtgccgttcaccctcaactttaccatcaccaatctgcagtatggggaggacatgcgtcaccctggctccaggaagttcaacaccacagagagggtcctgcagggtctgcttggtcccttgttcaagaactccagtgtcggccctctgtactctggctgcagactgatctctctcaggtctgagaaggatggggcagccactggagtggatgccatctgcacccaccaccttaaccctcaaagccctggactggacagggagcagctgtactggcagctgagccagatgaccaatggcatcaaagagctgggcccctacaccctggaccggaacagtctctacgtcaatggtttcacccatcggagctctgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggatctaggaagttcaacgccacagagagggtcctgcagggtctgcttagtcccatattcaagaactccagtgttggccctctgtactctggctgcagactgacctctctcaggcccgagaaggatggggcagcaactggaatggatgctgtctgcctctaccaccctaatcccaaaagacctggactggacagagagcagctgtactgggagctaagccagctgacccacaacatcactgagctgggcccctacagcctggacagggacagtctctatgtcaatggtttcacccatcagagctctatgacgaccaccagaactcctgatacctccacaatgcacctggcaacctcgagaactccagcctccctgtctggacctacgaccgccagccctctcctggtgctattcacaatcaactgcaccatcaccaacctgcagtacgaggaggacatgcgtcgcactggctccaggaagttcaacaccatggagagtgtcctgcagggtctgctcaagcccttgttcaagaacaccagtgttggccctctgtactctggctgcagattgaccttgctcaggcccaagaaagatggggcagccactggagtggatgccatctgcacccaccgccttgaccccaaaagccctggactcaacagggagcagctgtactgggagctaagcaaactgaccaatgacattgaagagctgggcccctacaccctggacaggaacagtctctatgtcaatggtttcacccatcagagctctgtgtccaccaccagcactcctgggacctccacagtggatctcagaacctcagggactccatcctccctctccagccccacaattatgncnnctgnccctctcctgntnccnttcaccntcaacttnaccatcaccaacctgcantangnggannacatgcnncncccnggntccaggaagttcaacaccacngagagggtcctacagggtctgctcaggcccttgttcaagaacaccagtgtcagctctctgtactctggttgcagactgaccttgctcaggcctgagaaggatggggcagccaccagagtggatgctgcctgcacctaccgccctgatcccaaaagccctggactggacagagagcaactatactgggagctgagccagctaacccacagcatcactgagctgggaccctacaccctggacagggtcagtctctatgtcaatggcttcaaccctcggagctctgtgccaaccaccagcactcctgggacctccacagtgcacctggcaacctctgggactccatcctccctgcctggccacacancnnctgnccctctcctgntnccnttcaccntcaacttnaccatcaccaacctgcantangnggannacatgcnncncccnggntccaggaagttcaacaccacngagagggttctgcagggtctgctcaaacccttgttcaggaatagcagtctggaatacctctattcaggctgcagactagcctcactcaggccagagaaggatagctcagccatggcagtggatgccatctgcacacatcgccctgaccctgaagacctcggactggacagagagcgactgtactgggagctgagcaatctgacaaatggcatccaggagctgggcccctacaccctggaccggaacagtctctacgtcaatggtttcacccatcggagctctgggctcaccaccagcactccttggacttccacagttgaccttggaacctcagggactccatcccccgtccccagccccacaactgctggccctctcctggtgccattcaccctcaacttcaccatcaccaacctgcagtatgaggaggacatgcatcgccctggttccaggaggttcaacaccacggagagggttctgcagggtctgctcacgcccttgttcaagaacaccagtgttggccctctgtactctggctgcagactgaccttgctcagacctgagaagcaagaggcagccactggagtggacaccatctgtacccaccgcgttgatcccatcggacctggactggacagagagcggctatactgggagctgagccagctgaccaacagcatcacagagctgggaccctacaccctggatagggacagtctctatgtcaatggcttcaacccttggagctctgtgccaaccaccagcactcctgggacctccacagtgcacctggcaacctctgggactccatcctccctgcctggccacacagcccctgtccctctcttgataccattcaccctcaactttaccatcaccgacctgcattatgaagaaaacatgcaacaccctggttccaggaagttcaacaccacggagagggttctgcagggtctgctcaagcccttgttcaagagcaccagcgttggccctctgtactctggctgcagactgaccttgctcagacctgagaaacatggggcagccactggagtggacgccatctgcaccctccgccttgatcccactggtcctggactggacagagagcggctatactgggagctgagccagctgaccaacagcgttacagagctgggcccctacaccctggacagggacagtctctatgtcaatggcttcacccatcggagctctgtgccaaccaccagtattcctgggacctctgcagtgcacctggaaacctctgggactccagcctccctccctggccacacagcccctggccctctcctggtgccattcaccctcaacttcactatcaccaacctgcagtatgaggaggacatgcgtcaccctggttccaggaagttcagcaccacggagagagtcctgcagggtctgctcaagcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttcacccatcagagctctatgacgaccaccagaactcctgatacctccacaatgcacctggcaacctcgagaactccagcctccctgtctggacctacgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccacgctcaggcccaagaaggatggggcagccaccaaagtggatgccatctgcacctaccgccctgatcccaaaagccctggactggacagagagcagctatactgggagctgagccagctaacccacagcatcactgagctgggcccctacacccaggacagggacagtctctatgtcaatggcttcacccatcggagctctgtgccaaccaccagtattcctgggacctctgcagtgcacctggaaacctctgggactccagcctccctccctggccacacagcccctggccctctcctggtgccattcaccctcaacttcactatcaccaacctgcagtatgaggaggacatgcgtcaccctggttccaggaagttcaacaccacggagagagtcctgcagggtctgctcaagcccttgttcaagagcaccagtgttggccctctgtactctggctgcagactgaccttgctcaggcctgaaaaacgtggggcagccaccggcgtggacaccatctgcactcaccgccttgaccctctaaacccaggactggacagagagcagctatactgggagctgagcaaactgacccgtggcatcatcgagctgggcccctacctcctggacagaggcagtctctatgtcaatggtttcacccatcggacctctgtgcccaccaccagcactcctgggacctccacagtggaccttggaacctcagggactccattctccctcccaagccccgcancnnctgnccctctcctgntnccnttcaccntcaacttnaccatcaccaacctgcantangnggannacatgcnncncccnggntccaggaagttcaacaccacngagagggtcctgcagactctgcttggtcctatgttcaagaacaccagtgttggccttctgtactctggctgcagactgaccttgctcaggtccgagaaggatggagcagccactggagtggatgccatctgcacccaccgtcttgaccccaaaagccctggagtggacagggagcaactatactgggagctgagccagctgaccaatggcattaaagaactgggcccctacaccctggacaggaacagtctctatgtcaatgggttcacccattggatccctgtgcccaccagcagcactcctgggacctccacagtggaccttgggtcagggactccatcctccctccccagccccacaactgctggccctctcctggtgccgttcaccctcaacttcaccatcaccaacctgaagtacgaggaggacatgcattgccctggctccaggaagttcaacaccacagagagagtcctgcagagtctgcttggtcccatgttcaagaacaccagtgttggccctctgtactctggctgcagactgaccttgctcaggtccgagaaggatggagcagccactggagtggatgccatctgcacccaccgtcttgaccccaaaagccctggagtggacagggagcagctatactgggagctgagccagctgaccaatggcatcaaagagctgggtccctacaccctggacagaaacagtctctatgtcaatggtttcacccatcagacctctgcgcccaacaccagcactcctgggacctccacagtggaccttgggacctcagggactccatcctccctccccagccctacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccattggatccctgtgcccaccagcagcactcctgggacctccacagtggaccttgggtcagggactccatcctccctccccagccccacaactgctggccctctcctggtgccgttcaccctcaacttcaccatcaccaacctgaagtacgaggaggacatgcattgccctggctccaggaagttcaacaccacagagagagtcctgcagagtctgcttggtcccatgttcaagaacaccagtgttggccctctgtactctggctgcagactgacctcgctcaggtccgagaaggatggagcagccactggagtggatgccatctgcacccaccgtgttgaccccaaaagccctggagtggacagggagcagctatactgggagctgagccagctgaccaatggcatcaaagagctgggtccctacaccctggacagaaacagtctctatgtcaatggtttcacccatcagacctctgcgcccaacaccagcactcctgggacctccacagtgnacntnggnacctcngggactccatcctccntccccngccncacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcaaccactggaatggatgccatctgcacccaccgtcttgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagagggttctgcagggtctgctcaaacccttgttcaggaatagcagtctggaatacctctattcaggctgcagactagcctcactcaggccagagaaggatagctcagccatggcagtggatgccatctgcacacatcgccctgaccctgaagacctcggactggacagagagcgactgtactgggagctgagcaatctgacaaatggcatccaggagctgggcccctacaccctggaccggaacagtctctatgtcaatggtttcacccatcgaagctctatgcccaccaccagcactcctgggacctccacagtggatgtgggaacctcagggactccatcctccagccccagccccacgactgctggccctctcctgataccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtctgagaaggatggagcagccactggagtggatgccatctgcatccatcatcttgaccccaaaagccctggactcaacagagagcggctgtactgggagctgagccaactgaccaatggcatcaaagagctgggcccctacaccctggacaggaacagtctctatgtcaatggtttcacccatcggacctctgtgcccaccaccagcactcctgggacctccacagtggaccttggaacctcagggactccattctccctcccaagccccgcaactgctggccctctcctggtgctgttcaccctcaacttcaccatcaccaacctgaagtatgaggaggacatgcatcgccctggctccaggaagttcaacaccactgagagggtcctgcagactctgcttggtcctatgttcaagaacaccagtgttggccttctgtactctggctgcagactgaccttgctcaggtccgagaaggatggagcagccactggagtggatgccatctgcacccaccgtcttgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctaacccacagcatcactgagctgggcccctacacccaggacagggacagtctctatgtcaatggcttcacccatcggagctctgtgccaaccaccagtattcctgggacctctgcagtgcacctggaaaccactgggactccatcctccttccccggccacacagagcctggccctctcctgataccattcactttcaactttaccatcaccaacctgcgttatgaggaaaacatgcaacaccctggttccaggaagttcaacaccacggagagggttctgcagggtctgctcacgcccttgttcaagaacaccagtgttggccctctgtactctggctgcagactgaccttgctcagacctgagaagcaggaggcagccactggagtggacaccatctgtacccaccgcgttgatcccatcggacctggactggacagagagcggctatactgggagctgagccagctgaccaacagcatcacagagctgggaccctacaccctggatagggacagtctctatgtcgatggcttcaacccttggagctctgtgccaaccaccagcactcctgggacctccacagtgcacctggcaacctctgggactccatcccccctgcctggccacacagcccctgtccctctcttgataccattcaccctcaactttaccatcaccgacctgcattatgaagaaaacatgcaacaccctggttccaggaagttcaacaccacggagagggttctgcagggtctgctcaagcccttgttcaagagcaccagcgttggccctctgtactctggctgcagactgaccttgctcagacctgagaaacatggggcagccactggagtggacgccatctgcaccctccgccttgatcccactggtcctggactggacagagagcggctatactgggagctgagccagctgaccaacagcatcacagagctgggaccctacaccctggatagggacagtctctatgtcaatggcttcaacccttggagctctgtgccaaccaccagcactcctgggacctccacagtgcacctggcaacctctgggactccatcctccctgcctggccacacaactgctggccctctcctggtgccgttcaccctcaacttcaccatcaccaacctgaagtacgaggaggacatgcattgccctggctccaggaagttcaacaccacagagagagtcctgcagagtctgcatggtcccatgttcaagaacaccagtgttggccctctgtactctggctgcagactgaccttgctcaggtccgagaaggatggagcagccactggagtggatgccatctgcacccaccgtcttgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaacagcatcacagagctgggaccctacaccctggatagggacagtctctatgtcaatggtttcacccatcgaagctctatgcccaccaccagtattcctgggacctctgcagtgcacctggaaacctctgggactccagcctccctccctggccacacagcccctggccctctcctggtgccattcaccctcaacttcactatcaccaacctgcagtatgaggaggacatgcgtcaccctggttccaggaagttcaacaccacggagagagtcctgcagggtctgctcaagcccttgttcaagagcaccagtgttggccctctgtactctggctgcagactgaccttgctcaggcctgaaaaacgtggggcagccaccggcgtggacaccatctgcactcaccgccttgaccctctaaaccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggttttcaccctcggagctctgtgccaaccaccagcactcctgggacctccacagtgcacctggcaacctctgggactccatcctccctgcctggccacacagcccctgtccctctcttgataccattcaccctcaactttaccatcaccaacctgcattatgaagaaaacatgcaacaccctggttccaggaagttcaacaccacggagcgggtcctgcagggtctgcttggtcccatgttcaagaacacaagtgtcggccttctgtactctggctgcagactgaccttgctcaggcctgagaagaatggggcagccactggaatggatgccatctgcagccaccgtcttgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccatcagaactctgtgcccaccaccagtactcctgggacctccacagtgtactgggcaaccactgggactccatcctccttccccggccacacagagcctggccctctcctgataccattcactttcaactttaccatcaccaacctgcattatgaggaaaacatgcaacaccctggttccaggaagttcaacaccacggagagggttctgcagggtctgctcacgcccttgttcaagaacaccagtgttggccctctgtactctggctgcagactgaccttgctcagacctgagaagcaggaggcagccactggagtggacaccatctgtacccaccgcgttgatcccatcggacctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccatcggagctctgtgccaaccaccagcagtcctgggacctccacagtgcacctggcaacctctgggactccatcctccctgcctggccacacagcccctgtccctctcttgataccattcaccctcaactttaccatcaccaacctgcattatgaagaaaacatgcaacaccctggttccaggaagttcaacaccacggagagggttctgcagggtctgctcaagcccttgttcaagagcaccagtgttggccctctgtactctggctgcagactgaccttgctcagacctgagaaacatggggcagccactggagtggacgccatctgcaccctccgccttgatcccactggtcctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccatcggacctctgtgcccaccaccagcactcctgggacctccacagtgcacctggcaacctctgggactccatcctccctgcctggccacacagcccctgtccctctcttgataccattcaccctcaactttaccatcaccaacctgcagtatgaggaggacatgcatcgccctggatctaggaagttcaacaccacagagagggtcctgcagggtctgcttagtcccattttcaagaactccagtgttggccctctgtactctggctgcagactgacctctctcaggcccgagaaggatggggcagcaactggaatggatgctgtctgcctctaccaccctaatcccaaaagacctgggctggacagagagcagctgtactgcgagctaagccagctgacccacaacatcactgagctgggcccctacagcctggacagggacagtctctatgtcaatggtttcacccatcagaactctgtgcccaccaccagtactcctgggacctccacagtgtactgggcaaccactgggactccatcctccttccccggccacacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccattggagctctgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggatctaggaagttcaacgccacagagagggtcctgcagggtctgcttagtcccatattcaagaacaccagtgttggccctctgtactctggctgcagactgaccttgctcagacctgagaagcaggaggcagccactggagtggacaccatctgtacccaccgcgttgatcccatcggacctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccatcggagctttgggctcaccaccagcactccttggacttccacagttgaccttggaacctcagggactccatcccccgtccccagccccacaactgctggccctctcctggtgccattcaccctaaacttcaccatcaccaacctgcagtatgaggaggacatgcatcgccctggctccaggaagttcaacaccacggagagggtccttcagggtctgcttacgcccttgttcaggaacaccagtgtcagctctctgtactctggttgcagactgaccttgctcaggcctgagaaggatggggcagccaccagagtggatgctgtctgcacccatcgtcctgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccattggatccctgtgcccaccagcagcactcctgggacctccacagtggaccttgggtcagggactccatcctccctccccagccccacaactgctggccctctcctggtaccattcaccctcaacttcaccatcaccaacctgcagtatggggaggacatgggtcaccctggctccaggaagttcaacaccacagagagggtcctgcagggtctgcttggtcccatattcaagaacaccagtgttggccctctgtactctggctgcagactgacctctctcaggtccgagaaggatggagcagccactggagtggatgccatctgcatccatcatcttgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacancnnctgnccctctcctgntnccnttcaccntcaacttnaccatcaccaacctgcantangnggannacatgcnncncccnggntccaggaagttcaacaccacngagngngtnctgcagggtctgctnnnncccntnttcaagaacnccagtgtnggccntctgtactctggctgcagactgacctnnctcaggncngagaagnatggngcagccactggantggatgccatctgcanccaccnncntnancccaaaagncctggactgnacagngagcngctntactgggagctnagccanctgaccaannncatcnnngagctgggnccctacaccctggacaggnacagtctctatgtcaatggtttcacccatcagacctttgcgcccaacaccagcactcctgggacctccacagtggaccttgggacctcagggactccatcctccctccccagccctacatctgctggccctctcctggtgccattcaccctcaacttcaccatcaccaacctgcagtacgaggaggacatgcatcacccaggctccaggaagttcaacaccacggagcgggtcctgcagggtctgcttggtcccatgttcaagaacaccagtgtcggccttctgtactctggctgcagactgaccttgctcaggcctgagaagaatggggcagccaccagagtggatgctgtctgcacccatcgtcctgaccccaaaagccctggactgnacagngagcngctntactgggagctnagccanctgaccaannncatcnnngagctgggnccctacaccctggacaggnacagtctctatgtcaatggtttcacccatcnganctctgngcccaccaccagcactcctgggacctccacagtgnacntnggnacctcngggactccatcctccntccccngccncacagcccctgtccctctcttgataccattcaccctcaactttaccatcaccaacctgcattatgaagaaaacatgcaacaccctggttccaggaagttcaacaccacggagagggttctgcagggtctgctcaagcccttgttcaagagcaccagcgttggccctctgtactctggctgcagactgaccttgctcagacctgagaaacatggggcagccactggagtggacgccatctgcaccctccgccttgatcccactggtcctggactggacagagagcggctatactgggagctgagccagctgaccaacagcgttacagagctgggcccctacaccctggacagggacagtctctatgtcaatggcttcacccagcggagctctgtgccaaccaccagtattcctgggacctctgcagtgcacctggaaacctctgggactccagcctccctccctggccacacagcccctggccctctcctggtgccattcaccctcaacttcactatcaccaacctgcagtatgaggtggacatgcgtcaccctggttccaggaagttcaacaccacggagagagtcctgcagggtctgctcaagcccttgttcaagagcaccagtgttggccctctgtactctggctgcagactgaccttgctcaggcctgaaaaacgtggggcagccaccggcgtggacaccatctgcactcaccgccttgaccctctaaaccctggactggacagagagcagctatactgggagctgagcaaactgacccgtggcatcatcgagctgggcccctacctcctggacagaggcagtctctatgtcaatggtttcacccatcggaactttgtgcccatcaccagcactcctgggacctccacagtacacctaggaacctctgaaactccatcctccctacctagacccatagtgcctggccctctcctggtgccattcaccctcaacttcaccatcaccaacttgcagtatgaggaggccatgcgacaccctggctccaggaagttcaataccacggagagggtcctacagggtctgctcaggcccttgttcaagaataccagtatcggccctctgtactccagctgcagactgaccttgctcaggccagagaaggacaaggcagccaccagagtggatgccatctgtacccaccaccctgaccctcaaagccctggactgaacagagagcagctgtactgggagctgagccagctgacccacggcatcactgagctgggcccctacaccctggacagggacagtctctatgtcgatggtttcactcattggagccccataccgaccaccagcactcctgggacctccatagtgaacctgggaacctctgggatcccaccttccctccctgaaactacancnnctgnccctctcctgntnccnttcaccntcaacttnaccatcaccaacctgcantangnggannacatgcnncncccnggntccaggaagttcaacaccacngagagggttctgcagggtctgctcaagcccttgttcaagagcaccagtgttggccctctgtattctggctgcagactgaccttgctcaggcctgagaaggacggagtagccaccagagtggacgccatctgcacccaccgccctgaccccaaaatccctgggctagacagacagcagctatactgggagctgagccagctgacccacagcatcactgagctgggaccctacaccctggatagggacagtctctatgtcaatggtttcacccagcggagctctgtgcccaccaccagcactcctgggactttcacagtacagccggaaacctctgagactccatcatccctccctggccccacagccactggccctgtcctgctgccattcaccctcaattttaccatcactaacctgcagtatgaggaggacatgcatcgccctggctccaggaagttcaacaccacggagagggtccttcagggtctgcttatgcccttgttcaagaacaccagtgtcagctctctgtactctggttgcagactgaccttgctcaggcctgagaaggatggggcagccaccagagtggatgctgtctgcacccatcgtcctgaccccaaaagccctggactggacagagagcggctgtactggaagctgagccagctgacccacggcatcactgagctgggcccctacaccctggacaggcacagtctctatgtcaatggtttcacccatcagagctctatgacgaccaccagaactcctgatacctccacaatgcacctggcaacctcgagaactccagcctccctgtctggacctacgaccgccagccctctcctggtgctattcacaattaacttcaccatcactaacctgcggtatgaggagaacatgcatcaccctggctctagaaagtttaacaccacggagagagtccttcagggtctgctcaggcctgtgttcaagaacaccagtgttggccctctgtactctggctgcagactgaccttgctcaggcccaagaaggatggggcagccaccaaagtggatgccatctgcacctaccgccctgatcccaaaagccctggactggacagagagcagctatactgggagctgagccagctaacccacagcatcactgagctgggcccctacaccctggacagggacagtctctatgtcaatggtttcacacagcggagctctgtgcccaccactagcattcctgggacccccacagtggacctgggaacatctgggactccagtttctaaacctggtccctcggctgccagccctctcctggtgctattcactctcaacttcaccatcaccaacctgcggtatgaggagaacatgcagcaccctggctccaggaagttcaacaccacggagagggtccttcagggcctgctcaggtccctgttcaagagcaccagtgttggccctctgtactctggctgcagactgactttgctcaggcctgaaaaggatgggacagccactggagtggatgccatctgcacccaccaccctgaccccaaaagccctaggctggacagagagcagctgtattgggagctgagccagctgacccacaatatcactgagctgggccactatgccctggacaacgacagcctctttgtcaatggtttcactcatcggagctctgtgtccaccaccagcactcctgggacccccacagtgtatctgggagcatctaagactccagcctcgatatttggcccttcagctgccagccatctcctgatactattcaccctcaacttcaccatcactaacctgcggtatgaggagaacatgtggcctggctccaggaagttcaacactacagagagggtccttcagggcctgctaaggcccttgttcaagaacaccagtgttggccctctgtactctggctccaggctgaccttgctcaggccagagaaagatggggaagccaccggagtggatgccatctgcacccaccgccctgaccccacaggccctgggctggacagagagcagctgtatttggagctgagccagctgacccacagcatcactgagctgggcccctacacactggacagggacagtctctatgtcaatggtttcacccatcggagctctgtacccaccaccagcaccggggtggtcagcgaggagccattcacactgaacttcaccatcaacaacctgcgctacatggcggacatgggccaacccggctccctcaagttcaacatcacagacaacgtcatgaagcacctgctcagtcctttgttccagaggagcagcctgggtgcacggtacacaggctgcagggtcatcgcactaaggtctgtgaagaacggtgctgagacacgggtggacctcctctgcacctacctgcagcccctcagcggcccaggtctgcctatcaagcaggtgttccatgagctgagccagcagacccatggcatcacccggctgggcccctactctctggacaaagacagcctctaccttaacggttacaatgaacctggtctagatgagcctcctacaactcccaagccagccaccacattcctgcctcctctgtcagaagccacaacagccatggggtaccacctgaagaccctcacactcaacttcaccatctccaatctccagtattcaccagatatgggcaagggctcagctacattcaactccaccgagggggtccttcagcacctgctcagacccttgttccagaagagcagcatgggccccttctacttgggttgccaactgatctccctcaggcctgagaaggatggggcagccactggtgtggacaccacctgcacctaccaccctgaccctgtgggccccgggctggacatacagcagctttactgggagctgagtcagctgacccatggtgtcacccaactgggcttctatgtcctggacagggatagcctcttcatcaatggctatgcaccccagaatttatcaatccggggcgagtaccagataaatttccacattgtcaactggaacctcagtaatccagaccccacatcctcagagtacatcaccctgctgagggacatccaggacaaggtcaccacactctacaaaggcagtcaactacatgacacattccgcttctgcctggtcaccaacttgacgatggactccgtgttggtcactgtcaaggcattgttctcctccaatttggaccccagcctggtggagcaagtctttctagataagaccctgaatgcctcattccattggctgggctccacctaccagttggtggacatccatgtgacagaaatggagtcatcagtttatcaaccaacaagcagctccagcacccagcacttctacctgaatttcaccatcaccaacctaccatattcccaggacaaagcccagccaggcaccaccaattaccagaggaacaaaaggaatattgaggatgcgctcaaccaactcttccgaaacagcagcatcaagagttatttttctgactgtcaagtttcaacattcaggtctgtccccaacaggcaccacaccggggtggactccctgtgtaacttctcgccactggctcggagagtagacagagttgccatctatgaggaatttctgcggatgacccggaatggtacccagctgcagaacttcaccctggacaggagcagtgtccttgtggatgggtattctcccaacagaaatgagcccttaactgggaattctgaccttcccttctgggctgtcatcctcatcggcttggcaggactcctgggactcatcacatgcctgatctgcggtgtcctggtgaccacccgccggcggaagaaggaaggagaatacaacgtccagcaacagtgcccaggctactaccagtcacacctagacctggaggatctgcaatgactggaacttgccggtgcctggggtgcctttcccccagccagggtccaaagaagcttggctggggcagaaataaaccatattggtcggaaaaaaaaaaaaaaa

SEQ ID NO: 3 - protein, 293 aa, Homo sapiens
  ref <308>: DATABASE ACCESSION NUMBER: GenBank / AAD26429
  ref <309>: DATABASE ENTRY DATE: 2000-06-27
  ref <313>: RELEVANT RESIDUES: (1)..(293)
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 4 - DNA, 1387 bp, Homo sapiens
  ref <308>: DATABASE ACCESSION NUMBER: GenBank / AF168768
  ref <309>: DATABASE ENTRY DATE: 2000-04-16
  ref <313>: RELEVANT RESIDUES: (1)..(1387)
ggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacttggggaaatcaggtgcagcggccatggctacagcaagacccccctggatgtgggtgctctgtgctctgatcacagccttgcttctgggggtcacagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaaagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaagtggaaaaaaaaaa

SEQ ID NO: 5 - DNA, 11570 bp, Homo sapiens
  ref <308>: DATABASE ACCESSION NUMBER: GenBank / AF135028
  ref <309>: DATABASE ENTRY DATE: 2000-06-27
  ref <313>: RELEVANT RESIDUES: (1)..(11570)
gggcccagagtgaaggcaagagaaggagttgagagctccctctgcaaagtggcttgagtctcccctgcctaaaatgcagggagagggaggcagaaagacagggaagaggaaggggtggggaagaaagagagagagagagagagacagaataacacaactacagaaacacagagagaacacacagagagcctgggacacagggacacacagagtcagagagaaaagagaagatagagaaagacacaaatggagacacagaggtgtaaagaaagagagattaacagagtcccagatacacgcaaaggggcagaagcacagttttcagggtggtgtctatgatcatcttcttttttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaatttttttgtatttttagtagagacggggtttcaccgttttagccgggatggcctcgatctcctgacctcgtgatccgcccgcctcggcctcccaaagtgctgggattacaggcgtgagccaccgcgcccggccatgatcatcttcttgactatgctgatgtgacaagtacctaaagccatcagactctaccctttaaatatgcagtttgggccaggcaccgtggctcatgcctgtaattccagcactttgggaggcagaggtgggtgaatcacttgaggccaggagtttgagaccagcctggccaacatggtgaaactctgtctttactaaaaaaaaaaaaaaaaaaaaaaaaatcagccgggtgtcgtggggcacacctgtaatcccagctatgctggaggctgaggcacgagagtcacttgaaccctggaggcggaggttgcagtgggccgagatcacatcaccgccctccagcctgggcgacagagcaagactctgtctcaaataaataaataaacaaacgaacaagcagtttgttgtaccttagttatatctaaaaaaaaaatgctgtcaacaaatagagcagaagtgaaataaaggaaaataaatgggccaagaactctaaggtatatttgacaaatcattcagaacctttaaaaaagaaagaatcacagaggcatagaaagacagggaggaacagggagacagaaacacctgtggcccaaggagaacaaaacaaggctcctaagacagacaggaggagagagagagagagtgagtgagagacagacagagaaaaagacagagagagagagacagagacagagagacagagaggcgagagggatagaaagagagagaggggtggagagagacacgagatattgagagagactcagaaagatagccgagggagaaccacagagagatggaagaagactctgagaaaaaaccagagacaaagatggaaagaggagtatcgagggtgaacagacagtggtggaatgagcaaaatgcagagaagaaagcaagcaatccaggcgccaagaatagtgacccagagttggtgagaagccagatccttaaggctgggggaggcagggaaggggctggcctggcttccggagacccctccccattctccgggccagggaggtagggagtgacattccggactgggtggggggtgctctgggggtggagatagggggagcaggaggagctattgctaaggcccgataggcacctcattgcccgggaatgtgccccagggagcagtgggtggttataactcaggcccggtgcccagagcccaggaggaggcagtggccaggaaggcacaggcctgagaagtctgcggctgagctgggagcaaatcccccaccccctacctgggggacagggcaagtgagacctggtgagggtggctcagcaggcagggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacctggggaaataaggtaggggagggaggggaagtgggttaagggctccccggatcgcctgggcctcccaaccctctgacattccccatccaggtgcagcggccatggctacagcaagacccccctggatgtgggtgctctgtgctctgatcacagccttgcttctgggggtcacaggtaaccagaactctggggtgggagggttgtgggattgggaggactgtctctgcggcactagagcgcctgtcccctggggaactgtgtgagcctgggcatgactccgggaccgggtgaatgtgagtctctgtctgtacttgtggttgtgcgatcgtatgtggccctgtgactgccacggtgtgtgtcggggagggggatgccttttcccatatcaggtgactgtgcggcaggtggcactgaccctttgaggctgtgtgtgtggttttgtgattgtgtgtgcatttaagattgtgtgtggctccacagctgtgtgggtgaatgcatgtagcactgggggtgttcactgtgtgtttggctgtgtgtggtgacttggcattgtatatgactgcaggtatctgcagttcctgtccctgaggtcccgggattgcgtgcaacaaaagtggtcatcaccatggaaagctgtgactgtgtgctgcttgcaggcgattatgtgattgtggctgagtgtgacgttatggatgcccgtatttgtgaccgtgtgactacctgaagctctgtgtaggggtgactgtatgtgactgtgtgtgtctgtgtgaggccgtgtaaatgctactgtatgtgtgatggtgcagctgtgtgtctggagtttctgtctctgcctggagggatagagggtgcaggggtagctatctctgggagatgggtgccaggtgactgacttgcagtgtgtgcctgtgtgcagaagagtatgtggcagtctgaacatctgtgcacacacggcatctgtgcgtggcactgagacactgtggatgagggtgtgcgatcccgctaggctgcccgggagcgtgtgtacctggagacagagctgtatgttagctgcacctgtggaggcaacatgggcgtgtctgcagaactgcgtgcgtgcttggctgttactgctgttgtgcgcgtggttcttggggtgagttcgtgaatgatggtggtgccagggccatcagcaagggtaagaaccaggccgggcgcggtggctcacgcctgtaatcccagccctttgggaggccgaggcaggcggatcacctgaggtcgggagatcgaggccagcctgaccaacatggagaaccccgtctctactaaaaatacaaaaaattagctggtgtggtggcgcgtgcctgtaatcccagctactcgggagactggggcagaaaaatcgcttgaacccgggaggtggaggttgcggtgagccgagatcgcgccattgcactccagcctgggcaacaagagcgaaactccgtctcgaaagaaaaaaagaaaaaaaaaagggtaagaaccagtgaatgggcacgggaggactgatgatggagtggggcatgcatgtagtctgtaggtctgtgtgtgagaggaggagattgacaggattgagaaggcatgttttcatctgagaattcagaaacctaggcctgctcttcccctccatgtggccccctaagctgagcccttctttcctggtcctgctttcggaaccctagctccgcccatgagctctgaccccacctcctttcctcaaccacgcccctaggccagactctagtggaccccgcctaaggccacacccctttgggccaggctccaccccctattctgtgggtaccttctagaacccccttcaaagtcagagcttttttttttttttttttggagacagtcttgctctctctcccaggctggagtgcagtggcgtgatctcggctcactgcaacctctgcctcccaggttcaagtgattctcgtgcctccacctcctgagtagctgggattacaggtgcgcgccaccacgcctggctaatttttgtgtctttagtagagacagggtttcaccttgttggccaggctggtctcaaactcccaacctcaggtgatccgcccacctcggcctcccagagtgctggggttacaggcgtgagccaccgcccccagcccaaagtcagagctctttataggagactctaacatgtaaccctgaccctggccctaactaagtcaattccaaaccccttcctgcctccagccctgaccccactcactgaggcctgaccccacttcttgagaccagttccatccctaaagccctggtctccctcccatccccaggctccagcccccacagctttggcactacccctgagcttgtccaggaatcctgtacccaattttaccctcacatgtagttctagccaattccaggaatctgtgaggtccagttagagtccagtaaccctacctgagcctgggctctgtccttgagcttgagcctgggcttgagaggtgccactcttattctccaggccctgcccctgccccctcagcatgtcagacacccaccctctagctggtctggcctcttgagtctgaaacccacccccagcccaagccccgcctctgagccccgcccaacccattttccgttcccagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaagtgagtgggagttccaagaggagggttggtggggacggggaagtgggggtgggggtggggaagtgggggtgggggtgtcatggaggtgagggctggtggggacggggaagtggggttgggggtgtcatggaaggtgagggttggtggggatgggttggggatgtgggagcaggaggaggtcgagttggggataggactaaggatggagttttgcgggggagcaaggtgggaggatgaggttggagaggggagagtgttgtggtagggaatgggaaggagccaaggatgggttggatttggggttaggagcatatatttgttgaatggtttgggatggaggtggaattgggattggctttagaattgggggtgggtgaaaatcgggctggggtggaaatgaagatagcatggagatagggttgagattgggagcagatatagaatgaaggatggggattggagttttgggtggggttggagatggttggatttgggcttgagaatgcatatggtgatggcttctgggtagggaaagaattagggttgggaatgggatgggtttggaattgtgactgggatggggacaggcatgggattggagaccaagagggagttgaggatggtttggggaccgggggtggggatgggggtggggctggggctgggtgtggggttgggattggcgttggacgtggagatagagatcagggttggtggtgacctgccccatcttcctcagagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaaggtgagtgtccaggttcttcttgataccgacccatctctgccgccttccatctttctccacttctcattgtgttcctgtttgacagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccaggtgaggacacctctctttattcagcagatacacactgagtgccaactcggtaacatggagcgttgccaaattctgagaatccagcaattgccaagacagtcaggacccctgttctcacagagctcataccctagagtagtggtgtttagtagaaataatgctgagctgcttatgtcatttccagttttttagtagccacattaaaacaggtaaaaaaggctgggcgcagtggctcacacctgtaatcccagcactttgggaggctgaggcaggcagatcacctttggtcaggagtttgagactagcctggccaacatggcgaaactctgtctctaaaaaaaaatacaaaaattagcctggcatggtggcgggcgcctgtaatctcagctgctcaggaggccgagacacaagaatcacttaaacccaggaggtggaggttgcagtgagctgagatcgtgccactcactccaacctgggagacagagtgacacttttgtctcaaaaagaaaaaaaaaaacaagtaaaaaagaaacaggtgaagttaactttaataacccaatgtatcccaaatacaatcatttcaaagtgtaattaatataaaacaattatgaatgagatactttacattcttttcttgttttcatattaagtctttgaaagtgagtatatatgttatgctgacagcacatctcaatttggactagctacatttcaggtgctcagtagccacatgtggctagcagttactgtattggatggcacggatctagagggaaagatcagggctgttttgtatggttgggcaggttgtgcactgcataaagataccatatctaataggggcactccgtgttacagatgtcagttttggcagttttcaggcgtgtggtagttaagtgtcttgtttcaacaaaatctgtaatatgacagttttctagcaagtgctggtaaaatatcttgaggaaggaaaagagaaatctggtaggtatttttacaagagaatatttaatacaggggattaattgcaaagctgctggaagggctggaggaacaaagttaaaaaataaaaaactctgtggtcaagaatctgcataaatagggcaatttcagagagtggtaaaggttaaccccaaaataaaacatggttttaggatagtaaacaataagggccaatattcaaaaaggtggtcaggggagcctccttggagaggtggcatttgagcagagaatggatgacacaaagaagctaaactcgtgaagtttaaggggaaagaaaaggcacgtgcaaaggccctgaggcagtaaggaatttggctgattcaaagaagaagaggaaaccaatgcaactggagaacaaaagtgggggcaacagtagaaagtgacgctggaggtgtaggcaggggcgaatgctctgcaagtatttcttggtcaccaacacagagcttccctatgttctaatggaagctgtatctgttgaggaagacagaatttaaaatcaaactgttacatcaaccagcacccttctctgtattcaggctcccaagggatctagaaggacgtaagttaacaagctctcattagcagggtgtgtgtttcaacagtagttaggaagctggggattcaggagtactccagtcccatggctatgaaaagctccccccaaattgtacaaacctgacaaatgcaacacctccccagctctccccatttcttctctgtgccctgggtgtgggggggtgggttgcgagggggaaaacttttaacagaagaaagcacatctcggccgggcgtggtggctcacacctgtaatcccaacactttgggaggccgaggcgggtggatcactaggtcaggagatggagaccatcctggctgacacggtgaaaccctgtctctactaaaaacacaaaaaattagccgggcgtggtggcaggcgcctgtagtcccagctactcgggaggctgaggcaggagaatggcctgaacccgggaggcggaacttgcagtgagccgaggttgcaccactgcactccagcctgggcaacacagtgagactccgtctcaaaaaaaaaaaaagaaaagaaaagaaatcacatctcattcaagtggtggcatttaaaactatttagcctttctgtaggcaaggttagtatcttgtttttccagacctcaaggtgtttttttgtttgttttttcataccggtgtgtggtctgggtgtggccactaaaagctacaagcaagaaataataacaactacaacaatactaataccaatagtataaaaataatagcatctggctaattgctggacactgttttaagtggtttgcatgcctcagctcattaactcatttacctgttattattggccctattttacaaacaaggagccaaggctcagagcagttaactaacagcctctcaaaagaaactctgcagagatattaaatttaaaaaataatgagagaaattaaaccacaagaaagttgaaatttagaggtacaggcagctaagcttgtttgctttgaaacagtgtctgctactgggaaaaaggcaagtcttggctttcctaataattgataccaggactctgtaattcatattttgcatgcatgtaagtaagaaatgaagccgggtgcaatggcacatgccagtaatcccagcactctgggagactgaagtgggaagatcacttgagctcaggagttcaagaccagcctgggcaactaaaaattaaaaaaataaaaatactaattgtttttattttagtagattttattcataccacttacatcattattgtagtatgtacatatttatttcttttcttttcttttcttttcttttttgagacggagtctcgctctgtcacccaggctggagtgcaatggcaccatatcagctcactgcagcatgcgcctcctgggttcaagcatttcttccacctcagcctcccaagtagctgggataacaggcacccaccaccatgcctggctatttttttttttccgtagagatggggttccaccatgttggccaggctggtcttgaactcctgacctccagtgatctgcctgcctcggcctcccaaattgctggtattacaggtgtgagccaccgtgcccaggtgggagatagacatttctctctacctcaaacagaggtccactcaagctacttttcattttcttcataaatattagccgagtggctattttgcaccaggaatggttccaggtgctgtggatatggcatcaggcaaaacagaccaaaaacttcctgccgcgtggacctcatgttccccaagtggaagacaggcaataaagagatagataaatatgtagtaaattaaaaaaaaaaaaaattagccgggtgtggtggcttgcacctgtagttccagctacttgggaggctgaggtgggagaattgcttgagcccaaacgtttgaggctgcggtaagccatgactgcactgctgcactccagacagcagcctgggtgacaaagcaagacgtttttgtcagaaagaaaaaaaaaagagacgaagggaggaaggagagagaaaggaaggaaggaaggagaaagaaaggaaggaaggagaaagaaaggaaggaaggaaggagaaagaaaggaagaaagagaaagaaagaaaaagaaagaaagaaagaagaaagaaaagagagaggaaggaaggaaagaaggaaaagagggaaaaaaatgactgttgaagagcagtgagtattattataggagggtaattatagggaggtatggggaattgaagacaggaaacacaaattagtccaagcgaatggatttctattgggagtgattctgcccctagaagacactggcaataccaggagacatttttggttgtcacaactatatggaggggcattactggcaactaatggatagatgccaagtgtgctgttcaacatgctatgatgcacacggcaggcctccacaacaaaccattatccagcttcagatgcccacagtgcccagatcgaggaaccctcatccaggggctgagaaccgtatttttgcagaagggaggtataaggatgggttggtggagaatggggaaggaaggtgtgtgtccagtaagagaaataaggcctgcacaggctggaggggagagtgagagagaaagggaggcggagagatacacgatgagggagacaggctggaacagaaagtagagacgaagattcgagatgtggagaggaagggtcacagacccccccgaaatgatgtgtggacaacaggaatctggaagaggaagatggagtggagagtgacaaatggggtctaaaggttgaacttggaggccaggcatggtggctcacgcctgtaatcccaacactttggaggctgaggtgggcgaatcacttgaggccaggagttcgagaccagcctggccaacatggtgaaaccccgtctctacaaaaaaaatacaaaaaattagccgggtgtggtgatggacacctgtagtcacagctacttgggaggctgaggcaggagaattgcttgaacccgggagatggaggctgcagtgagctgaggtcaggccactgcgctccaacctgggcaacagagtaagactccatctcaaaaaaaaaaaagctggatttggagtgaaatattaataacattctccctctctctccttttgcctgtgtctccatctctgtctttttctgcatttcttcatctctgtactttccatctctgtgtgtctgttcccatctgcttctccatctatgggcatctctgggtctctcatgtctccttctgcccactttgccacatctctgcctctctcatgcccccctttctctcctgcagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaag

SEQ ID NO: 6 - protein, 244 aa, Homo sapiens
  ref <308>: DATABASE ACCESSION NUMBER: GenBank / AAB66483
  ref <309>: DATABASE ENTRY DATE: 1997-08-12
  ref <313>: RELEVANT RESIDUES: (1)..(244)
MKKLMVVLSLIAAAWAEEQNKLVHGGPCDKTSHPYQAALYTSGHLLCGGVLIHPLWVLTAAHCKKPNLQVFLGKHNLRQRESSQEQSSVVRAVIHPDYDAASHDQDIMLLRLARPAKLSELIQPLPLERDCSANTTSCHILGWGKTADGDFPDTIQCAYIHLVSREECEHAYPGQITQNMLCAGDEKYGKDSCQGDSGGPLVCGDHLRGLVSWGNIPCGSKEKPGVYTNVCRYTNWIQKTIQAK

SEQ ID NO: 7 - DNA, 1451 bp, Homo sapiens
  ref <308>: DATABASE ACCESSION NUMBER: GenBank / AF013988
  ref <309>: DATABASE ENTRY DATE: 1997-08-12
  ref <313>: RELEVANT RESIDUES: (1)..(1451)
gtcgacccacgcgtccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaattttattatgaaaaaaaaaaaaaaaaaaaaaaaaaaaagggcggccgc

SEQ ID NO: 8 - DNA, 12483 bp, Homo sapiens
  mRNA (2001)..(2185)
  mRNA (3084)..(3135)
  mRNA (3559)..(3606)
  mRNA (4346)..(4502)
  mRNA (8122)..(8369)
  mRNA (9791)..(9927)
  mRNA (11805)..(12483)
  ref <308>: DATABASE ACCESSION NUMBER: GenBank / AF149289
  ref <309>: DATABASE ENTRY DATE: 2000-06-26
  ref <313>: RELEVANT RESIDUES: (1)..(12483)
acacttaaaaaatcttctgacttaaaaaaaaaagtatggtgattggaaaatgtaaatgtgcatgcgtgcttggcatcacatttcattggccaggacttccctggatgctaaaggtcctcaaatgccaggctggggggctgggacttggtcccaagggagatggggacccagggcacgtctgtgagaggaggggcaaggtcagcacaaggcacaggaaggtctctctggggcaagggatacagagaacagagggatcctggtccaggtgggagaggtgcagctctgagttggggttgagggtgtgggtacagagaggaagggaccccccagagagaggaggcagagggatagggcctggtcactgggttgtgcaacatcagacttgctgtctgtgaagatagcacgtcctgagaagaaggtgctgaggtcagtggggaccaaatgtgagagggagcacccggagagtatactgaataccgaagtagtcttcatccctggagtgatggggggtgcacaatgcaagatgacaattagattcaatgcaagacaaagaaaagggttggctgggaacagtggctcatgcctatggtcccagctcctgggaagactgaggcgggagggtcgcttgagcccaggagggttgaggctgccacgagcaaggatcgtgccactgcactccagcctaggcgacagaacaagaccttgtctcaaaagaaaaaagaacttttttttttaagttacctgtagtgcccagcccaagcaggtgctgagccagacttcattcctatcattgtccttattacgcagtgacttccccctcctcatttctctccactctgccacgcacacaccctcaccctccagcccataccaaccaccccaaccactgcctgtggtttcccatgtgcacccaggccaggcattttcacggcctttcctcctgacctacgcctggctcagctttctaggcccaagttcaaagacacctccctaaatcttcccagatccctctgctactgcccagcaccaccatcttatcacagccccacgtcgttcccaagtgctctccgatttctgcttaactccatgcctctcgctgtgtgtccgcatctcatcaataagtcctcaagtcctcttccatcctgctagcttcctcatcgctcgggaatcatccccgctacttcctggggaaactgactcccttctgggcacacacagtgctacccccggggaaatctaagaagagacccaggagaagataagcacggagagtcagagaatcaaggggaaagaaagggagagaggccgggcacagtggctcacacctgtaatccagcactttgggaggccaaggtgggtggatcacctgaggtcaggagtttgagaccagcctggccaacatggtgaaacctcttccctactaaaaatacaaaaaacatttagccgggcgtggtggtgggtgcctgtaatcccagctacttgggaagctgaggcaggagaactgcttgaactcaggaggcggaggttgcagtgaactgagatcacaccactgcactccagcctgagtgacagagcaagactccgtcaaaaaaaaagaaagaaagaaagaaaagaaggaaggaaagaaagaaggaaggaaggaagggaggaagggagagaggaagggagagaggaagggagagagagaaaaaaagagggagagagacacaaatacagagactgagatgggagagagagagagatggaagctccctcccctccatggccagggagacagatggagcaagagacctcaggggtgggcagacttggaggagaaggaccaggaggatgtggagtgccgaaatctccagtcagggccaggtgggcagtcagagactgcaaaggaggactgtcagacagggacaaaaggaagccattgatgtaaccgccctcccgcctgcccgccggaagagaggttgaggccggagctgctgggagcatggcactggggtgctgggaggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcaggtgtgtgagtcaccccaaccgcactgaacctgggcaggctgcttcccagtgccggagggctctagagcccggagtgagggcctgcaggtccctgggtggcacagagagtgctgggggtgcagggaggcctggggcaccatctgcttgccccagaggccggaatttgtcttcagacactttctttctccaaaacccggaggtctaaggactgagccgactagaacttcctctgcctcagattcaggccccagcccctcctccctcagacccaggagtttaggtcctagcccctcctccctcagacccaggagtccaagttcccacctcctccctcagactcaggagtccaggcccccagcccctcctccctcagacccaggagtccaagttctcacctcctccctcagacccaggagtccaggccccaagcccctcctccctcagacgcaagggtccaggcccccagcccctcctccctcagactcaggagtccaggcccccaagcccctcctccctcagacccaggagtccaggccctcactgcactcagggaccagtgctcccttccctggaggcctggtcaggggtcaccaagagcagagcgtgggggcgggaggaatgtgtgtgggaggcctgggtaaggaggaaaagggtgtagccagtctcctggctcagggacctgagagacaggggttaaaaggacgttccagaagcatctggggacagaaccagcctcttccagggaggcctgggagctgggggtgtgtgtctggcagtccctgcagccctgggctctgcggcccctgcgtcctccgcttggctctgccactgcatctgagtgtcttctctcctcacggctccccgcatttctaactctttctgcctcctcgtctcaaagctgttccttcccccgactcaagaatccccggaggcccggaggcctgcagcaggtgagatcacagacatcacagaacctgccgggtgggcggggtgggtggccattgcgcacagagccaggctccgaggaaaactcccatacagaggaagaacgctagggccccctatggtaaccctctcctgtcgacaggaaggcaaatcagtgcccaagaaagtagaaagatctaatcagaatctcaccatgggttactggaccagtggacgtagttgaattctctttggcactgttttcgtggatcctcttggaagatgtgggctgaggaagaataaatcaggaggctagatgggaaggacagaggtcaaggcaggagaccatagcaggccaggaaggaaggagaggatgcagagggagcagacagagggatggggggagggtcgaggcagtgactaatggaccatgtggcttcccctctcaggagcggccatgaagaagctgatggtggtgctgagtctgattgctgcaggtggggaaagggcatttggatgggggaggcttgcagacagggttgggcttgttgatggagaagaggctggtattggggatggggatatgcacagggttggggtgggggagctttgaaatgaggaagacgttggggattaggctaagggtggggaatacagatagggagggtggtgggaggtgggtttgaagatatgagggtttggggtggggttggctttagggatggggatctaaacatagaagaggtaggaggtaggttggaaagttggagagagcccgggaataggggatacagttgggtttgtaatgggaatggggtaagtttgggagtggaaatacagagaagctttttttttttgagacagggtctcactctgtcacccaggctggagtgtagtggcatgatccatagttcactgcagacttgaactcttgggtctcaagtgaccctcccacctcagcctcccaagtagctgggactacaggcgtatgccaccataccctgctaatttgtgtgtgtgtgtgtgtgtgtgtgtgtgtgtgtgtgtgtggagatgaggtctcactgtgttaccgaggctggtctcaaactcctgggctcaagcgatcctcctgcctcagctgggattacaggcataagccactgcacctgaccaatcttgactggagttcatgttgagggggatgcgcttggtttctccagaactcctctctgactcagatcttctctccctcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgtgagtctacactgtaaatgaacagcagatgcgactgaaccctgagggtgtcttatagatgtcaggcaggaggtgacataggcatcccccccatcccagcacgaggccatctgatagccaggtgcattcggctgttgcttaattgagtacttaatgtgtgccaggccctgcgggcatagcagtggaaaagaaaataaaaaaaagaaaacaaaaaaaaacaagcaaaattgctgttttcctgaacttactttctaatgggggaattggatcatttggggacctgcagggcgtgatgggcatttggatttaattctgagcacagtaggaagccactgggcagttttgtttttgttgtttgtttgttttttgagacacagtctcgctctgtcacccaggctggagtgtagtggcatgatctcagctcactgcaacctctgcctcccaggttccagcgattctcctgcctcagcaccccaagtagctgagattacaggtgtgcaccaccttgcctggctaatttttgtatgtttggtagagacggggtttcaccatgttggccaggctggtctcgaactcctgacctcaggtgatccgcccgcctcgccctcccaaagagctgggattacaggcatgagccaccaccacacccagcctgatttacatttttacaagcaccctggctaccacgtggaacgtggtctgggcaagagagagggagggaggcccacgtgggggctgttgctttcatccggcgacataggagggtggcttgaacccaggcggtcgcagtggggatggagggatgttgaatatcttgggatgtggaattctgagactgagccagcagaatctggcaacgaggaacaggagggagaggaagaagcacggctggcttccgtgtatttgtcctgaacaactgggtgttttgccacgtctttctctgagttgtgggagagggaaagagaaacaggccgggtgtaggcaggggagcatctgacattttgctttagccacgatgagttggagatgccggggagatgtcccagcagggaggccagggaggactctggagctcagaggagaggtcagggctggaggttaaaatgaaggcatcgtcagcaaacaggtgtatttaaagccatgggactagatgagatcatccaaaaagctggcatagttggaggagctggagggcccaggacaaaaaccctgggcgctgatcctcactagtcagattcacgacagctgccacttgtttgatgctaactaccaatcaggtgctgagtgaaaccatgtacacacctttcctggaatgcccaccacaagggactcttggcaccattttgcaaatgaggaaactgaggtgcagggaaatagcaagtgacaatccctggggtggttcccctgaccccaaggagaccttggatgactctcaccaccatcattcattcctttgatgtacattgactaagagcacctgctaagtgccacattcgagttgggcagtggagattcagcaatggatgggacacacacgtcatccctgccctcgggagcacaaggacagaaaggtgcagacaagcaaagtgagggctgggcatggtggctcacgcctgtaatcccagcactttgggaggccgaggtgggtggattacctgagttcgagaccagcttggccaacatggctcaaccctgtctctactgaaaatacaaaaaattagccaggcgtggtggtgggcttctgtaattccagcaacttgggaggctaaggcaggagaattgcttgaacgtgggaggcggaggttgcagtgagccgagatcgcgccactgcactccagcctgaaccacagagcgagactctgtctaaaaaaaaaaaaaggaaagaaagaagcagcaaattgggctggccgtggtggctcatgcctgtaatcccagcactttgggaggccgaggcgggtggatcactcgagcccaggagtacaaagctgcagtgagctgtgatctacagaacaccactgcagatccagcctgggtgacagagcgagaccctgtctcaaaaaaacaaacaaacaaaagaagcaaacccttcaaaaccccatataattacaaattatgaaggaaaagaatacgggtacctactttagatggaggagggtcaggaaggactttctaatgagataaaatccaagcggaggcatgaagatgggaaaaggaatgttcagggcagaggaaaggctgtgataacacccctgaggtgagaaccgtcttgagtattctcagaaaataaaatttcccgttcactggggggcagaaggtgctgggagataaggttggaaagtgactacagccagatcacacaggggctccagtgccaagtggaggagcccaggctttattcttaggacaatggggagccatgggtgatgtctgagcaagggagtgactctctgtttcaggaatatgtatcaaacacctatcctgtgccaggtgctgatcaacgcactggagatactatatctgaatagaacaaaaatccccatcttgacatcctagagctgcactgtctaatatggtagccatcagccacatatagcaaattacattgaaattaatgaaatggaaaatccacaagccacatttcaagtactcagcagccacctgtagcttgtggttcccccagccacctctggacagtgcagatcgagatcatggcatcgtagcatttagtggacagcattgctctgcaaggaggagaaataacacaatgagtaaatatttaacaataaatatatagcaggtcggatgattgtgataggttctctggtggaacagaaagcaggggagggagataggaattgcctactaacaggtatttgtattttaattgggcaactaaggaaggcttccctgagaggcgacatttaaaggaagtgagggagtgagctatgcagatacttggaggacagacttgctggcagagggaacagcagtgcaaaggccctgaggtgggaagatcactattgtgttcaaggcaagacagggaagccagcgtttggctggagcagagggagagaaggggagagtgggaggagaagatgtctgtgagatgatggggcagtgcttgcaaggcctggtgtgccacgttgagaactttggctttgattctgagtgagatgggagtcataggaggggctgagcagaggaggcacaggaccaacttacattgttaaaatatctctggttgctttgtggaggatggactgtgggggaccagagacagagcagggagcccagtgaggaggctactgctctagttcaggtaggaagtgaaaaggcagctcaaaccaagatggtagccgtgggaaaggtgagatgtggccagattctggatatgcttcagagaggcaaaaggaattctggacagcttggatgtagggcatgaaataaagagagtgaagaatagcccccaagattattctgaaaggatggaattgccatttacccagctggggaagactgtgggaggagcaggccagcgattcatgacttcccagccctctctgaagcctcaactgcagcccaagggctccaggtgagacccagccctcttccttcccaggaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtcagtagtgggaggctggtggggagcaggctactggctacttggggaagtgtgccaaaggatggggagtgggaaaattggtgaggggccatgggaagatgggctaatggtgaggaccaatgggacagggtttcaatgggagaaaggtcaagggggagggagagtgaatttgggagctgggccagtgagtgaacagccaatggaaaatgtagaccaatgggtgaatagcatgggagagatggaacataagatgaaggttcaataaagagggaaggtcagtggggagatgctaatcaggaaggatgtcaaaggtcaaaggggactgatcaggattcattgaacagcaggaaggaataatggagaaggaactgatggaagaagagaaaccaataaagcacaaaagccaactgaaggatgtgaattgagacagtgaatgggggtatagctgatggaagagggactaaggggaaaggatcaatggtccagaggagtcactagaggaaaaaacaggtccaatagatcagcaggatccatgaaggtgggcctgtgtgtgaagggccaataagaaaggtgaaccattggatgaagggccagtgggaaggcagagacaatgggggaggatgcggcaagttagaaaaggaccaatgagggaggtggaccattggatgaagggctaataggaagggagagccagtgggggatggtgaggccagttagaaaaggaccaaggagggaagcagaccaataggaagagagagccaatgagggagggcagggccagttaggaaaggaccaatgaggaaggtagaccattggaggaagggccaatagaaagggaggatccatgagggagggtggggacagttagaaaaggaccaatgatggaggtggaccattggatgaagaaccaatagaaaggaagaaccaatgggagagggcatggccagttaggaaaagaccaatggtcacagagtgaccaatcaagatgaatcaatgggcaggaagtgtccaatgaagaatggactactgatcaggaggggtacagtagaggagggcgtaacagaggaagagtcctccaggtcaactgaaactactgaagaaggtgggaccagtggaagagagaaaagtggaggagggacctaagagaaaaggaaaaccaataggaaatgaggactcctggagaagagactattaatgaggaagacagccaatgggggggaagaatgatagaaagagggaccaattaggaggcagggacgatggtaatgagatgtaagaatgagagacaaacaggaagaggggtgccaatagaaaagagggaccaatagaggatggaggacttataggggttggggggtgactggggaggatgaggggagtgcaaggcctgggctgagtctggcccatctctcccctaacaggtgatttccctgacaccatccagtgtgcatacatccacctggtgtcccgtgaggagtgtgagcatgcctaccctggccagatcacccagaacatgttgtgtgctggggatgagaagtacgggaaggattcctgccaggtgaggtgacccggatctgccacttacacagccagggacaggacgaagtcacaaaaacatggccagacacaggaagagagagacacaggccaaaagagagctttacagagacagatagagacaggctgagggagaacccaagccttgaaaagaagagacttagttcaacacacagagacacagtcagggatatgcagagatataaagacacagccagcagagacaggaagtgcagagacaaggatggaggccgcgggatcaagaaccagagaggccaggagcagcggctcatgcctgtaatcccggcactttgggaggccgaagcaggaggatcacctagggtcaggagttcgagaccagcctgatcaacatggtgaaaccctatctctactaaaaatacaaaaattaggatgggcacagtggctcatgcctgtaatcccagcaccttgggaggccgaagcaggaggatcacctggggtcaggagttcgagaccagcctgatcaacatggtgaaaccctatctctactaaaaatacaaaaattaggatgggcacagtggctcatgcctgtaatcccagcaccttgggaggccgaagcaggaggatcacctggggtcaagagattgagaccagcctggccgatatggtgaaaccctatctctactaaaaatacaaaaattagctgggcctggtgcaggcgcctgtagtcccagctactcaggaggctgtggcaggagaatcacttgaacctggaggcggaggttgttgcagtgagtcgagatcatgctactgcactccagcctggcaacagagcaagattccgtctcaaaaaaaaaccaaaaaacaaaaattacgcaagcatggtgggacacacctgtagtcccagctactcgggaggctgaggctggagaattgcttaaacccaggaggcagaggctgcagtgagctgagatcacgccactgcactccagcctggggacagagccagactctgtctaaaaacaaaaagaaccaaagagaagtagtaaggaagcagatggtgtgaggggactgtccttcctcaaacagagcccccacgagtcctgctcagaaacgaccaggctctggaggagggagacactagctggggaaaggggactccctcccgaatactttaacttgggtttcctccattgtcatccatccaggctctcctctttatgccagaatgactaatgcactgagggatgtgcagagaccaaccaagggggagacacaggcagaaacggagacacaggcagaaacagggacagagacagggaaagcgatacatagcaagttggacgcaaagaaagggcaggtgggcgagactgtcctcaagacacgaggtggagaggtgtccctggacagaatagtgccaggcatatctctccctgggccctccctacctctcccacctgggtcttatcgtctcctcctccccctcctccctctcctcctcttcctcctcctcctcctccctcatcatcttcttcttttctctctctctccatcggtctctacacctctgcctctctccacacctctcagtctccattcttaaattgtttctctttcttgctctctatgttcctctgcatcttggcattcctatctctgtgtctttgagtctcctttattctctctctaccattctctctctgtgcctttgtgtgtcttactgtctctctctctgtctctctgtccctgagtctttctctccatctttcagtaagtacctctgtccctttctacctctctctctgtcacacacacacacacacacacacacacacacacacacacacacacagtctctgggtttctatctgtatctgactttctccctctttcctgcagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccataaggcagtggctgttggaaagatttaaggtttcacacctatgacatacatggaatagcacctgggccaccatgcactcaataaagaatgaatttt

SEQ ID NO: 9 - DNA, 1506 bp, Homo sapiens
  ref <308>: DATABASE ACCESSION NUMBER: GenBank / U62801
  ref <309>: DATABASE ENTRY DATE: 2001-05-15
  ref <313>: RELEVANT RESIDUES: (1)..(1506)
aggcggacaaagcccgattgttcctgggccctttccccatcgcgcctgggcctgctccccagcccggggcaggggcgggggccagtgtggtgacacacgctgtagctgtctccccggctggctggctcgctctctcctggggacacagaggtcggcaggcagcacacagagggacctacgggcagctgttccttcccccgactcaagaatccccggaggcccggaggcctgcagcaggagcggccatgaagaagctgatggtggtgctgagtctgattgctgcagcctgggcagaggagcagaataagttggtgcatggcggaccctgcgacaagacatctcacccctaccaagctgccctctacacctcgggccacttgctctgtggtggggtccttatccatccactgtgggtcctcacagctgcccactgcaaaaaaccgaatcttcaggtcttcctggggaagcataaccttcggcaaagggagagttcccaggagcagagttctgttgtccgggctgtgatccaccctgactatgatgccgccagccatgaccaggacatcatgctgttgcgcctggcacgcccagccaaactctctgaactcatccagccccttcccctggagagggactgctcagccaacaccaccagctgccacatcctgggctggggcaagacagcagatggtgatttccctgacaccatccagtgtgcatacatccacctggtgtcccgtgaggagtgtgagcatgcctaccctggccagatcacccagaacatgttgtgtgctggggatgagaagtacgggaaggattcctgccagggtgattctgggggtccgctggtatgtggagaccacctccgaggccttgtgtcatggggtaacatcccctgtggatcaaaggagaagccaggagtctacaccaacgtctgcagatacacgaactggatccaaaaaaccattcaggccaagtgaccctgacatgtgacatctacctcccgacctaccaccccactggctggttccagaacgtctctcacctagaccttgcctcccctcctctcctgcccagctctgaccctgatgcttaataaacgcagcgacgtgagggtcctgattctccctggttttaccccagctccatccttgcatcactggggaggacgtgatgagtgaggacttgggtcctcggtcttacccccaccactaagagaatacaggaaaatcccttctaggcatctcctctccccaacccttccacacgtttgatttcttcctgcagaggcccagccacgtgtctggaatcccagctccgctgcttactgtcggtgtccccttgggatgtacctttcttcactgcagatttctcacctgtaagatgaagataaggatgatacagtctccatcaggcagtggctgttggaaagatttaagatttcacacctatgacatacatgggatagcacctgggccgccatgcactcaataaagaatgtatttt

SEQ ID NO: 10 - protein, 253 aa, Homo sapiens
  ref <308>: DATABASE ACCESSION NUMBER: GenBank / AAC37551
  ref <309>: DATABASE ENTRY DATE: 1994-08-26
  ref <313>: RELEVANT RESIDUES: (1)..(253)
MARSLLLPLQILLLSLALETAGEEAQGDKIIDGAPCARGSHPWQVALLSGNQLHCGGVLVNERWVLTAAHCKMNEYTVHLGSDTLGDRRAQRIKASKSFRHPGYSTQTHVNDLMLVKLNSQARLSSMVKKVRLPSRCEPPGTTCTVSGWGTTTSPDVTFPSDLMCVDVKLISPQDCTKVYKDLLENSMLCAGIPDSKKNACNGDSGGPLVCRGTLQGLVSWGTFPCGQPNDPGVYTQVCKFTKWINDTMKKHR

SEQ ID NO: 11 - DNA, 969 bp, Homo sapiens
  ref <308>: DATABASE ACCESSION NUMBER: GenBank / L33404
  ref <309>: DATABASE ENTRY DATE: 1994-08-26
  ref <313>: RELEVANT RESIDUES: (1)..(969)
ggatttccgggctccatggcaagatcccttctcctgcccctgcagatcctactgctatccttagccttggaaactgcaggagaagaagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaatgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatgtgacctttccctctgacctcatgtgcgtggatgtcaagctcatctccccccaggactgcacgaaggtttacaaggacttactggaaaattccatgctgtgcgctggcatccccgactccaagaaaaacgcctgcaatggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaacctcatgccctgttgataaaccaatcaaattggtaaagacctaaaaccaaaacaaataaagaaacacaaaaccctcaa

SEQ ID NO: 12 - DNA, 9909 bp, Homo sapiens
  ref <308>: DATABASE ACCESSION NUMBER: GenBank / AF166330
  ref <309>: DATABASE ENTRY DATE: 2000-09-18
  ref <313>: RELEVANT RESIDUES: (1)..(9909)
ggcatggtggtgcacgcctgtaatccagctactcaggactctgaggcaggagaatcacttgaacacgggggagtggaggttgcagtgagccgagatcgtgccattgcactccagcctgggtgacagagccagagtccatcaaaaaaaaaaaaaaataagaaagattcttctctcctctatgtgtccatgcagtctcatcatttagctaccacttgtaagtaggaacatgccatatctggttttctgttcctgctttagtttgtaagggtaatggcctccagctccattcacgtccctacaaaggacatgatcgtgttcttttttatggctacgtagtattcaattgtgtatacgtaccacattttcttaatccagtctatcactgatggacatttaggttgattccctgtgtttgctgttgtcaatagttctacaatgaacgtacgtgtccatgtgtctttaaacagaatgatttatattcctttgggtacacacactggggcttatgagagggtggagagtgggaggaaggagaggatcagaaaaaaataactaatgggtactaggcttaatacctgggtgattaaataatctgtataacaaacccccatggcgcacgttcacctacgcaacaaacctgcacatcctgcacatgtacccccgaactgaaaagttaaaaaaagaaaaataaatatttgcttataaattaataaatgaagccctcaaaaatgttctattagataatgttaagtacagacatttttgttataaatacataatatacaaagaaatctatgtataacatgattaaaatgaccataagaacatagatcctaaacatggcaaatattagtggggtggggttagggaaagcgttgtttttaacttacacctctctgttagagttgggaatgggttcaggcgtaattacaggcacgactgggatcagcttggacaagttcccccaggcgggccagaattaggatgtagggtctaggccacccctgagagggggtgagggcaagaaaatggccccagaagccgggcgcagtggctcacgcctgtaatcccagcactttgcggggccgaggcgggcacatcatgaggtcaggagatcgagaccattctggccaacatagtgaaacccggtctctactaaaaatacaaaaattagctgggagtggtggtgcgtgcctgtaatcccaggtactcgggaggctgaggcaggagaatcacttgaacctgggaggcggagctggcagtgagccgagatcgcgccaccgcactccagcctggcgatagagagagactccatccaaaaaaaagaaaggaagggagggagggaggagggaagaaagaaagaaaaccgccccagagaaggacccgagccagagcctattctctgagctcagcgactgcttgaatcccgctcctgcccctcagacccagcgcaccgggtccctcccccgagagcagccaggagggactgtgggaccagaatgtgcgggggcgcaggagctgggcaccgcccgtccttcggagggagggtggagagagagtgcagtggtgccaattgctctcgctgcgtcagggttccagataaccagaaccgcaaatgcaggcgggggtgtcccagagtcggctccgcctgcaccccagggcgctggggccgggcatggggcggggggtgatataagaggacggcccagcagagggatgaagattttggagcccagctgtgtgccagcccaagtcggaacttggatcacatcagatcctctcgaggtgagaagaggcttcatcaagggtgcacctgtaggggagagggtgatgctggctccaagcctgactctgctctcgagaggtaggggctgcagcctagactcccggtcctgagcagtgagggcctggaagtctgcaatttggggccttttagggaaaaacgaactacagagtcagaagtttgggttccacagggaagggcaagatcggagcctagattcctgggtctctagggatctgaagaacaggaattttgggtctgagggaggaggggctggggttctggactcctgggtctgagggaggagggcctgggggcctggactcctgggtctgagggaggaggggctgggggtctcgactcctgggtctgagggaggaggggctgggggcctggactcctgggtctgagggaggaggggctgggacctggactcctaggtctgagggaggaggagctggggcctggactcctgggtctgagggaggaggggctggggcctggactcctgggtctgagggaggaggggctggggcctggactcctgggtctgagggaggaggggctggggcctggactcctgggtctgagggaggaggggctggggcctggactcctgggtctgagggaggaggggctggggcctggactcctgggcctgagggaggagggactgagacctggactcctaggtctgagggaggagggactgggacctggactcctgggtctgagggaggaggagctgggggcctggactcctgggtctgagggaggaggggctggggcctggactcctgggtctgagggaggaggggttggggcctggactcctgagcctgagggaggagggacttggacctggactcctaggtctgagggaggaggagctgggggcctggactcctaggtctgagggaggcggggctgggggcctggactcctgggtctgagggaggaggggttggggcctggactcctgagcctgagggaggagggacttggacctggactcctaggtctgagggaggaggagctgggggcctggactcctaggtctgagggaggaggggctgggggcctggactcctgggtctgagggaggaaggtgctagggtctggactcttgggtatgagggaggaggaggttaggggtctggacttctgagtgtaaggaaggagaggccagagaaaggaatttctgggtctgagggaggaggggctggggttctggacccctaggtctgagggaggaggggctggggcctggacccctgggtctgagggaggaggggctggggccggtactcctgggtctgtggggggaggggctggggcctggacccctgggtctgagtggggaggggctgggcctgaatgctttctccttctcagctccagcaggagaggcccttcctcgcctggcagcccctgagcggctcagcagggcaccatggcaagatcccttctcctgcccctgcagatcttactgctatccttagccttggaaactgcaggagaagaaggtgaaagctggactgggaagtctgacctcacctcagggcccccactgaccctctccaaggagtccctgagtcagaacccttccctcctcaaacagcttccatcctgggaggaccagactgtcggctgaagcccccgctcttcctgcttctgctgactcagggggtctctgtcccctccaggccctgcctcctgtgctcagggtctctctgtggttccccagatgagatgcgcctcctgggtttctgagtgggctccttctgtctgtctctatccctatctcttgctttctctgtatttctccacacattttcatctgtctctgtccatctctgactctgggaatccctgaggtgcagcctcagccttcccctaatgctagctacccacgtgctcctccatgtctccatccagcccagggtgacaagattattgatggcgccccatgtgcaagaggctcccacccatggcaggtggccctgctcagtggcaatcagctccactgcggaggcgtcctggtcaatgagcgctgggtgctcactgccgcccactgcaagatgaagtaggtgccgcccaagtctctgctggaggtgcaccagcgtctccagctcgctatgggggtggaagggcagtctttctgtgcctacggctctattctcctctctctgggtctctgtcctcctctctctgggcctctgtaccccctctccctggggctctgtccccctctctccctggctctctgtctccctctctctgggtctctgtccccctctctctggatctctgttcccctctctctgtgtctctgttccccattctctctaggtctctgttccccctcctctctctctgggtctctgtccctctctctctggatctctgtccccctctctctctgggtctctgttcccctctctctgggtctctgtcccctctcctctctctgtgtctctctcccccctcctctctctgtgtctctgtcccccctcctatctctgtgtctctctcccccctcctctctctgggtctctgtccccccctctctgggtctctgtctccctctctctggggctctgtccccctctctctctggatctctgttcccctctctctgggtctctgtctcccctcctctctctgtgtctctgtcccccctcctctctctgggtctctgtccccaccccgtcccccaggtctttgcacaccctctctgtcacagtgtctcttctgaatctgtgaatgtcactcctcgcagtgagtacaccgtgcacctgggcagtgatacgctgggcgacaggagagctcagaggatcaaggcctcgaagtcattccgccaccccggctactccacacagacccatgttaatgacctcatgctcgtgaagctcaatagccaggccaggctgtcatccatggtgaagaaagtcaggctgccctcccgctgcgaaccccctggaaccacctgtactgtctccggctggggcactaccacgagcccagatggtaggtggcctcagtgacccaggagtgcaggccccagccctcctccctcagacccaggagtccaggcccccagcccctcctccctcagacccaggagtccaggcctcagcccctcctccctcagacccaggagtccaggcccccagcccctcctccctcagacccgggagtccagaccccagcccctcctccctcagacccagcagtcctgggccccagaccctcctccctcggaaccaggagcctgaacaacagcccttctggtcctcgcccccatcctctctgactgacagctctccctgctcctccctgcagtgacctttccctctgacctcatgtgcgtggatgtcaagctcatctccccccaggactgcacgaaggtttacaaggacttactggaaaattccatgctgtgcgctggcatccccgactccaagaaaaacgcctgcaatgtgagaccctcccccccaattcctccccagtcctgggtaccctgtctgcatgccccagggacagagcttgacccgagtgactgggtaccaagcccggccttgccctccccccaggcctggcctcctcagctttttccacctcattctctgcctaggtcaggggtgggagtttacttaggggccaatgtggccctggggatgagacagagagtttaataggggtgagaaagtgggggtgggaccagggaaggagactgaggtgctggcctcaggcccaaaccttaagggggcaccaaaaacctcagtgattgagataaatcataatgcaatatttaaaaataaaaataaaaactcatgcagaagtccatgatggacaaaatgtcacattttaaataaagagcaggtggatcttactgaattttcccttgccgtaagtactagcgtggctcagcacagcgctgtactggcactgtcttcatttaaaatgtggataccatgcccatcatgcagttttatgtattacatttgatttcgttaagtactgcattgaagtattgtgtattgcagttactgagattttgtgcctgaagctgatgactcactcacctgaccctggccctggtcccggggaaaacactctttctctccacctcctctctgttccctctttctggccttttgtcatcccctctgtttctgaacagtcttcccacatctctctttgtgacataatttcatttcattcttttcctctttgttttttctctgtgttgagctagcttgctctccctcccttgttctctctccatgccctcctctctgctctctgtcttctccctctttctcttgcttctctctctctcctcccctccctctctcctctccctgcccccctgctctctcttttttcctctctctctgtctcctctctggccctctcctctttctctctctcccccacttctctgtctctcttcatctctctccctcatctctccttgccccctcctttttactgtctctctctttctctttcttctatctctctcctctccctgccgctcccccatctctgtctttctttctctctctttattctcctcctctcttccagtctctctctcctctccccacccccaccccatctctctccccacaccttccccccctttctctttgtctctctcttctccctctttcttctccacccccatctctctctctcttctcttcccacaccctccccatctccctcatctctttgtctgtctctcttctccctccttcttttccacccccatctctctgtctctctctctccccataccctttccctcttcctcatctctctttgtctctctctcctttccctctttcttctccacctccaactctctctgtctctccacacccatcctccttgctcacatctgcaccttcagctgtcaggggatgtgggatggtgagtgttagggatagaggagatgggagagagatgactgtcctagagaatagggtgttccccttctcccctggtgagggccagtttcatgaatgtgcaagctctgcacggacacagagccccacactcagaagggtctcaaacttagtctaatgcattcctgctgttgtcttgaaattctcaataatttttgaacaaagggccctgcattttcgttttgcaccaagtcctgtaaattatgtaactggtcttcaccctggtctccgagaccatcgtgtccccctttcctgcgccacagggcacgcatccaccccttggagatgatgttccttctcccactagcttggagcagggtccttaacattggaaaataaagagtgctctgatcctggaagccccaccccttctctgcaattggtctcattggccaagggtcaaaccagtgtcttcaaaggacctagtgtgtccctagcactagctctcccattagtccccagagacaatgagtctcttctcattggctatggtggaagtccataatctgcaagacaaagaccgataactgaggaatgtatgagaatgagttgggctttgatctgaagccaaagttaatctccggctctattccctctagggtgactcagggggaccgttggtgtgcagaggtaccctgcaaggtctggtgtcctggggaactttcccttgcggccaacccaatgacccaggagtctacactcaagtgtgcaagttcaccaagtggataaatgacaccatgaaaaagcatcgctaacgccacactgagttaattaactgtgtgcttccaacagaaaatgcacaggagtgaggacgccgatgacctatgaagtcaaatttgactttacctttcctcaaagatatatttaaaccaacctcatgccctgttgataaaccaatcaaattggtaaagacctaaaaccaaaacaaataaagaaacacaaaaccctcagtgctggagaagagtcagtgagaccagcactctcaaacactggaactggacgttcgtacagtctttacggaagacacttggtcaacgtacaccgagacccttattcaccacctttgacccagtaactctaatcttaggaagaacctactgaaacaaaaaaaatccaaaatgtagaacaagacttgaatttaccatgatattatttatcacagaaatgaagtgaaaccatcaaacatgttccaaaagtaccagatggcttaaataatagtctggcttggcacaacgatgttttttttctttgagacagagtctctgttgcttgggctgcaatgcagtgatgcaatcttggctcactgcaacctccgcctcctgggttcaagtgattctcgtgcttcagcctcccaagtacctgggactacaggtgtgcaccaccacaccaggctaattttttgtgtatttttactagagacagggtttcaccatgttggccagcatggtcttgaacgcctgacctcagatgatccacccaccttggcctcccaaagtgctgggattacaggcatgagccaccacggccagcccacaatgatattacaaacctattaaaaatgatacttagacagaattgtcagtattattcaagaacatttaggctataggatgttaaatgacaaaaggaaggacaaaaatatatatgtatgtgaccctacccataaaaaatgaaatattcacagaatcagatctgaaaacacatgtcccagactgcatactggggtcgtcatgaggtgtctccttccttctgtgtacttttccttgaatgtgcacttttataacatgaaaaataaaggtggggaaaaaagtctgaagatctaagattggagagaggtgacctttcaggaagggagactagaaagaaatatgtgcctggttttgagccctggtcctgccggccctgttccagggcatatttccatttcccagatctcagtttttcctgtctgtaaaatgggagagagaggaaaggatggagagaggaagaaggaagggaggagggaggagagaacaggccaacttcatcagcgtgggaaggggtgtgaaagtgtttctgagcatctcacgagtgacaagtgaggagggaggctggcggttttcagagggattgggatgacagtagacaggacacaggggtcccacgggggtctgccagaagtaagcaaacagtgccggaggaagatggtggcacctgctccccaagaagggagggaaaggaacctcgggaagcgggtaggatgagggaggagtcctctgtgactcagagcctggccacagccccagccatctaacatcaaagatcctctgtgtggtcacacctcagacgctgctgaccgaggagccactccagcccaggacacgccctcctacctgttcttcctgtttttctcccagaattccctccccaccaagatcctccagatccttcccctccttatctcatctccctctgagtctctcctaacccaggcaccacagccctgtcatattgcagaaattctgcagccgctaattctgattctcccatataggaggctaacacagaaaacgcaggagtccaggcccccagcccctccttcctcagacccaggagtccagaccccccgccccaacccctcctccctcagacccaggagcccaggtccccagccccttctgtttctgggcctgtcaagtttaagaatgtcaaacattttcgaccagtcattcccctgaagttttagcaacattttctctctcttctgcaaggcactccaacattcaatctggaattttaaaaagtaacaaaacattgcatttgcactaagtcagcctggagatccctggccctggccctctgctctcctatacgcaagctacaggtagattggtttgcaatgactgagatggtactaatgttgattttttttaagtaattcatttttctttgggtaagcagtatagtgtggtagttaagggactagctctggatcttggcttcttgggttcaaatcccagttctagtccctacaagctattttcctttaagctcattacttcccctgtccctgttccttcatccttgaaatgggagaaaaagcacctactttctagggttattacagagattcaataagttaatatacagaaagtgctcaaacattgt

SEQ ID NO: 13 - protein, 260 aa, Homo sapiens
  ref <308>: DATABASE ACCESSION NUMBER: GenBank / BAA28673
  ref <309>: DATABASE ENTRY DATE: 2003-01-31
  ref <313>: RELEVANT RESIDUES: (1)..(260)
MGRPRPRAAKTWMFLLLLGGAWAGHSRAQEDKVLGGHECQPHSQPWQAALFQGQQLLCGGVLVGGNWVLTAAHCKKPKYTVRLGDHSLQNKDGPEQEIPVVQSIPHPCYNSSDVEDHNHDLMLLQLRDQASLGSKVKPISLADHCTQPGQKCTVSGWGTVTSPRENFPDTLNCAEVKIFPQKKCEDAYPGQITDGMVCAGSSKGADTCQGDSGGPLVCDGALQGITSWGSDPCGRSDKPGVYTNICRYLDWIKKIIGSKG

SEQ ID NO: 14 - DNA, 868 bp, Homo sapiens
  ref <308>: DATABASE ACCESSION NUMBER: GenBank / AB009849
  ref <309>: DATABASE ENTRY DATE: 2003-01-31
  ref <313>: RELEVANT RESIDUES: (1)..(868)
gtgaccccgcccctggattctggaagacctcaccatgggacgcccccgacctcgtgcggccaagacgtggatgttcctgctcttgctggggggagcctgggcaggacactccagggcacaggaggacaaggtgctggggggtcatgagtgccaaccccattcgcagccttggcaggcggccttgttccagggccagcaactactctgtggcggtgtccttgtaggtggcaactgggtccttacagctgcccactgtaaaaaaccgaaatacacagtacgcctgggagaccacagcctacagaataaagatggcccagagcaagaaatacctgtggttcagtccatcccacacccctgctacaacagcagcgatgtggaggaccacaaccatgatctgatgcttcttcaactgcgtgaccaggcatccctggggtccaaagtgaagcccatcagcctggcagatcattgcacccagcctggccagaagtgcaccgtctcaggctggggcactgtcaccagtccccgagagaattttcctgacactctcaactgtgcagaagtaaaaatctttccccagaagaagtgtgaggatgcttacccggggcagatcacagatggcatggtctgtgcaggcagcagcaaaggggctgacacgtgccagggcgattctggaggccccctggtgtgtgatggtgcactccagggcatcacatcctggggctcagacccctgtgggaggtccgacaaacctggcgtctataccaacatctgccgctacctggactggatcaagaagatcataggcagcaagggctgattctaggataagcactagatctcccttaataaactcacaactctctggttc

SEQ ID NO: 15 - DNA, 6500 bp, Homo sapiens
  exon (1)..(39)
  exon (418)..(712)
  exon (878)..(946)
  ref <308>: DATABASE ACCESSION NUMBER: GenBank / AF095743
  ref <309>: DATABASE ENTRY DATE: 2001-06-11
  ref <313>: RELEVANT RESIDUES: (1)..(6500)
tcttcggttcccggttactggcagcagccccctcctcccacaaaagatcaggttccaagcttctccttttaaaagtacttagaattcagcccccagctctctcctccctcacacccaggaatccaggcccctagcccctcctccctcagacccaggagtcctggcccctagcagccccctcctccctcagacccaggagtctgggcccccagcccctcctcggtcagacctaaatcccaggtcccagtccctcctcccttagatttaggagtccaggcccccagcctctcctccctcagacccaggaatccaggcccccagcctcctcccctctcagaactaaaatcttggcccccagccctttatgtttcagatcgtagagtctcagcaccgagtccctcctctccctagcctcaggagtctgagattccagcccctcctccctcaagatttcacgttcaatcccctccgccccttctcactcacacccagtgttccagttcccagaagctccccaggctctagtgcaggaggagaaggaggaggagcaggaggtggagattcccagttaaaaggctccagaatcgtgtaccaggcagagaactgaagtactggggcctcctccactgggtccgaatcagtaggtgaccccgcccctggattctggaaggtgaggtgcagaggtactcagatacagacatcaggccccggaccctccttctccagattccaggaccccagcctcagatgcccttctctgtcgagatccagcagtctggaccccggcttcctcctctccctaatttaggagtcccagctcccagctccctgtcccctcagacccagacatcgaggactcccccctcccttggaatgtaggaatccagtcccccagcctcctccttcctccagagaagcccagaacagccccagatactctcggctgcctccccagtgcccaaatccagaactgggagctcaggctcctccttcctgtttaccggccccgccctctccatttcccagacctcaccatgggacgcccccgacctcgtgcggccaagacgtggatgttcctgctcttgctggggggagcctgggcaggtgaggagggttgcggaggcctccggaggggagggatctgaaggcagcagtggcgctggggagtctgtgggaatgccgcgggggttatgtgggtgcgtgtgcacggatgtgaagagtgcgatacggtgcaggagcctctgtgggctttcctcagggtggacagaggcaagaaacaggtagcagcaggtaggagtaggttccgtgatgctgtaaattgtctgaatagctacagcctttgggggctgcttgcttgggggcatagattcacctgggagtactcggggcctgtagactcatgtggaagcatgtgggggcattcttgggtgtgtgactcttgtatgatgacacatggactgaaatgagtgtccccgtgtggcagcgtgtggaagcctggacctcctcactaagttgtatgcggagaacttgccgtgtgtccatttgaacccacagtggccttcccagccctcgcactgccccagagggtggcgatccaaccctctccctcctgctgcaggacactccagggcacaggaggacaaggtgctggggggtcatgagtgccaaccccattcgcagccttggcaggcggccttgttccagggccagcaactactctgtggcggtgtccttgtaggtggcaactgggtccttacagctgcccactgtaaaaaaccgtgagtggatgatgggggcagaggtcagctggggcttaaggaaagagggggctggggtttcgactcaggaaggagagagctgaggactggactcctgggtctgaaggaggagggggctgggggcaatacccctgcctgggtcccaaactatccccaccattacaggaaatacacagtacgcctgggagaccacagcctacagaataaagatggcccagagcaagaaatacctgtggttcagtccatcccacacccctgctacaacagcagcgatgtggaggaccacaaccatgatctgatgcttcttcaactgcgtgaccaggcatccctggggtccaaagtgaagcccatcagcctggcagatcattgcacccagcctggccagaagtgcaccgtctcaggctggggcactgtcaccagtccccgaggtagtgggcttgtccactaatgggagggagaggaggagctggttggcccagtggaacccaagctattggcaaagcttggtcccccagagggagacaaagaagggaaagtgatcatgatgttgagattcacaagcaggagtcatatgagaagcctcgaagatctgactactaacaagagtggtgagagaaagaaccaactagcagattgttaagcagggccagaaaagcccatcctgtatggcggagagcactaccctatgggacctttggttgtataggattttatataatcttaacacacttgggatattttggacttctcagaggcccaggaaaacaggctcctaagcaccttctcccccacctccctccttttttttttttttttttttttttttgcagggggacacagtttcactcaatcgcccaggctggagtgcagtggcacaatctcagctcactacaatctctgcctcctgggtttaagcgattctcgaacctcagcctcccgagtagctgggattacaggcacccgccaccacgcgagctaatttttgtatttgttgtagagacagggtttcgccatgttgaccaggctggtctcgaactcctgacctcaagtgatctgcccgcctcggcctaccaaagtgctgggattacaagcatgagccaccgcccctggcctttttctccttcttgaacccaggaaacctgggccctggtcacctcttccctcagacccgggagtctaggcccttcttctcccaggacccagcagtcctagctccctcttgactctggaccccaaaatctggacctccaatgaagctgtccctttgggactccagaatccagagagcccctcgccttccttcacagtgaaaacattgggactcacctaagagagtcaaggagcttctccaggaagtggcaaagtcagcattcaggtccctgcctgcctcactcctgctctgaatgctttggatgagacagtttgcggctgtggaaacacacgtgctcgcaaatcaagtagatcagttcaaactatggctctgccctttctccctgggcaaattcctttccatctctgagcctcactttccttatctgcaaaatgggaatcattaaccaatagattttttagctacgtgagggttcaggttacatcagttttcttcattgtgttctcctagtgctcagaatggtgcctgctacatggtagatgctcaataaatatttgttgaatggatgacctgatgaataaataatgaaatgaatgaatgcatctctccttcaaagcgctgttgtgaggattaaatgagattatgagcatattgcttttggcacgtagagatgccagatggaagtttttccccctcaaagaggctttggagaagtctattcctcaaaagaggttaataaaaaagatcaattccaccttcaatcattaattcaactcttatttactgagcacctagtatgcctgaggtgctgttgcaggcgctggatatacagccatgagcaaactgtacaaagtccttgtcgttatggagttgcaagctaggtgggagaaatagacaataaacaaatacacataaaataaaacattaggcaaagtgctgtaaggaaatattgtggagcgtaaaaggatagggagtaatggagggatggtattttttaattcgagtggtcagggaaggcttccaggaggaggtgacacttggaaggagcaggatttagcacagattgaggacccggttgtctgagggcaggaagactggaaagagagggtcagaggtatcataaaggggcacctccaagtaacccccagccccttgatttgaaaatggctcagggtaagaaaaaacacgtgagatccaagggcccctctctagatggagaaagcccaatagcaacaagtacagcttcgtttaatgtggtgagaagtgatgtcccctgtgcacagtgtcagaaaattcccccatgcagctggaaaactcccccattacatcctggaaagaaagggggttagatccagacagggatggaggcaaagggctgctctctcagggaaccttacaacctcttccccctcagagaattttcctgacactctcaactgtgcagaagtaaaaatctttccccagaagaagtgtgaggatgcttacccggggcagatcacagatggcatggtctgtgcaggcagcagcaaaggggctgacacgtgccaggtgagcaatttctgaaatccttctcctcacacatccctcattgccctctcgaggttcaaggcttggatgggggtgggggtggtaagggagtgaccccaaagacttggcacccggagtgttcacccctatctctacggattgggagccaggttcagagaagccaaactctctctctgaaagtcacactgcatagggattaggagcattgaattgctgctgctgttttctttcgaacgcttaactacaggatgggatgcagagttgggggctatagagggtggggtagatgtccagcaaggagagagtctgaggctagagtttagacgagttgcctgctctctggttcccagcattacatcgtggaatttgtgccgactttaccaccggcccggtggtcagccacttttctttgagaccgggctagaatcccaaggctggttcccttctcctgtgattggttccttgggagacaatggtgtcttcccagttggctggagtaaatggtgccattgacttcagtgcttcctcaatgagctccaatccctttctcttgggatttgtcatagatataactctctcttctaactgcaacttcgttgttcctagcactttcccctggctttgtccacttcctggaagccccaccacctttgccaatgactggtccctaaatacaatgctttcttccccattggccaaaaatggagtcgtttccatcagcgatactgccatgaaagccagtctctggattgttctgtagagatagtggtctcttccacaaatatttcagccatggtctcttggggatatggtgtgtgtgtgtgtgtgtgtgtgtgtgtgtgtgtgtgtgtgtgtgtccataagaatcttgatcctttctcctatgtttggcaactgcaatcaatgatgccttcactattggccaggaacagaggaaacttcagctcagtcctctccaagtaatccctactgtcttctccctggattggaccctcgagaactcttttttttttttttttgagacagggtctcgctctgtcccccaagctggaatgcagtggcacaatcttggctcactgcagcctctgcctcccagttcaagcaattctcccacctcagcctcccgagtagctgtgattacaggtgtgcaccaccacacccagctagtttttgtatttttagtagagacagggattcaccatgttggccaggctggtctggaacgcctgacttcaagtgatctaccgcctcggcctcccaaagtgccgggattgcaggtgtgagccaccaagcctgtctgggatttcattctttcccctcttctgtcagtgttttgaccactacccttagacaccatgtctgtctgtacatggaagccccaagccctgtcctgactggtctcaggggacaatgcttttacccccattggctacaggggaccaatcatgccaaagaactggtaaaacgctgggacagcaggaaaagggacgttgtggacatctcagatgcaaggctgttctcattctccctgtctagggcgattctggaggccccctggtgtgtgatggtgcactccagggcatcacatcctggggctcagacccctgtgggaggtccgacaaacctggcgtctataccaacatctgccgctacctggactggatcaagaagatcataggcagcaagggctgattctaggataagcactagatctcccttaataaactcacaactctctggttccttgcctgtctctgttttggccctgtggggagggctggatggggatccgggattgttcctgctggcagactaggtggggatgtgcagaaaccaagttctcatggtcactttggccatcaccactgcctaaagtgatccctcgttttctggaagaacttgggtaagagctttatttcaggggagaaaatacatacaaaggtcttcaaacattcagtgggctggtatgtgaaagacagttttgaaagagtttgtgtttagttttcctgagcaaagcatttacaagctttggagataaaattttccccctttaaaaaataatt

SEQ ID NO: 16 - protein, 276 aa, Homo sapiens
  ref <308>: DATABASE ACCESSION NUMBER: GenBank / AAC14266
  ref <309>: DATABASE ENTRY DATE: 2000-08-30
  ref <313>: RELEVANT RESIDUES: (1)..(276)
MRAPHLHLSAASGARALAKLLPLLMAQLWAAEAALLPQNDTRLDPEAYGAPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVPGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

SEQ ID NO: 17 - DNA, 1580 bp, Homo sapiens
  gene (1)..(1580)
  ref <308>: DATABASE ACCESSION NUMBER: GenBank / NM_002776
  ref <309>: DATABASE ENTRY DATE: 2003-12-21
  ref <313>: RELEVANT RESIDUES: (1)..(1580)
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtga

SEQ ID NO: 18 - DNA, 5574 bp, Homo sapiens
  gene (1)..(5574)
  promoter (1)..(47)
  mRNA (48)..(120)
  5′UTR (48)..(120)
  exon (48)..(120)
  mRNA (605)..(701)
  5′UTR (605)..(613)
  exon (605)..(701)
  mRNA (2455)..(2635)
  mRNA (3589)..(3863)
  mRNA (4195)..(4328)
  mRNA (4793)..(5474)
  ref <308>: DATABASE ACCESSION NUMBER: GenBank / AF055481
  ref <309>: DATABASE ENTRY DATE: 2000-08-30
  ref <313>: RELEVANT RESIDUES: (1)..(5574)
ttggggtcaaaagggaaggtcccgccaggggtccctgggcagaggataccagcggcagaccacaggcagggcagaggcacgtctgggtcccctccctccttcctatcggcgactcccaggtgaagctacctgcaccccacccgggttggggtggattgcgagaggatgggtgggaacccccgggccacaggcaggagccggcttagagcctcggtttctccactgcgggacgcggaagtccccccgctgtgaggttgagaaagaggctcccactggctccgagcctcggatccccaccccgcgcgtgaaggagggggaaacctcgggcgcggcgtggctgcagccagggtagctggggcgcggagagcgctccactcgggcacagggaggacgggaagatgccgcgaggggcgtcattagggtaattgtgcccattaccgtgttccagcccgaatctcccgtctgccagcccctggggttatcggctgcaggtcaaaagggtgcttgcgggctggggtggcccaggctgggcggatgagcgcggcgaggtgggggtctctaacggagcatctgttttaacccgcccctgcacacaccccagcagatcctggccatgagagctccgcacctccacctctccgccgcctctggcgcccgggctctggcgaagctgctgccgctgctgatggcgcaactctggggtaaggtgggggacagggggcggggagaggcgccggtgggaggcacgggcgggagggcaatgtgttcccgtcaccaagccccgcgcacctctcctcccccgagaccccagcacccacccagcgctccggagcacggcccgcccccaagtcagctgggcccttcttctggctcggcccctgggtgacccgccccactcaggccctgtccgatttctgccacccggatcctcgctcttccgtggactcttcggcgtgttctttctcctcctcttacgccgccccatcccggccccgctccattttacactaccgtgttttgttttgtttgtttgtttgtttgagacggagtctcgctctgtcgcccaggctggagtgcagtggcgcgatctcggctcactgcaagctccgcctcccgggttcacaccattctcctgcctcagcctcccgagtagctgggactacaggcgcccgccaccaagcccggctagttttttgtatttttagtagagatgggatttcaccgtggtctcgatctcctggcctcgtgatccgcctgcctcggcctcccaaagtgctgggattacaagcgtgagccaccgctcccggccacaccacagtgttttatcctgagtcttgccttaccgctttttgccctctcccctcactttttttcttcctctttccctttctctctcttttttctttttctttctttcttttctttctttcgttcttcttttccttccttcctttccttccttcctttcgtttgtttcttctttcttgttttactttctctcttattttttcttctttctttcttgttttttttctttctctctctttttctttccttctttttttttttttttttttgagacagggcagtgctctgtctccgtggctggagtacagtggcccaatcagagctcactgcagcctcgacctcctgggctcaagcgatactcagcctccagagtagctggtaccacaggcatgcaccaccacatccggctttttttttttttttttttttttttttttttgagacagggtctcactctgtcgcccagactggagtgcagtggcccaatctcggctcattgcaacctccacctcctgggctcaagcgatcctcccacctcagcctcccaggtagctgtgactacaggcgcatgcctccgcgactacttttttgttgttgttgttgtttgtttgtttttgtagagactgggtcttgctgtgttgcccgggctggtcttgaattcccgagctcaagcggtccaaccgcctcggcttcccaaagtgctgggattacaggcgtaaaccactgcgccccacccctctcctggttttcaatcccgttttgttattcacaccccttcctctccccgatccccgagttctatccccgcacccttacctccccgccgcgttcaatccccgcccctctatcgaccagcgacgttctagccagctctccaggcgcgctgcgttcagtccctgccctccagacccaccctattctgtctcattactccacgctaccctatcccagcttccttccactttcacgcgcttcttctcctcccattccttcggtgcacgcgaaacccccaatatttccctaccaccctcgcgttctggctgcgtcccccgtccccgaacgcagtccagtgccacagcccagctccaaccccaaacccagaccctgccctccgtgctttgattccgtcccctttctttctcccagccgcagaggcggcgctgctcccccaaaacgacacgcgcttggaccccgaagcctatggcgccccgtgcgcgcgcggctcgcagccctggcaggtctcgctcttcaacggcctctcgttccactgcgcgggtgtcctggtggaccagagttgggtgctgacggccgcgcactgcggaaacaagtaggaggagatccatccccgaggacgccacggggggctgtggaggcggcctccagggaggagcgggcagggcggggtctctcggaactcccacagctggaggtgcggtccccggtgtccttccagcaggaggagagaccgggcttgcgctgtggccgccaggggacggtgtggtccttttccgcatttctgggcccgtcactcctctcccgctgattctccttgagctctaggaggaggtggttgcctgcaacggatagaagccagggtccggtgtggtcaggaatttagaactaaaggaaaaggttcacttcgtgagtccccgttgaaggaggaagggttgggtattaccacagagaaaatgtggagttgggctgggctcggtggctcacgcctgtaatcccagcactttgggaggccaaggcgggtggatcacctgaggtcaggagttcaagaccagcctggccaacatggtgaaaccacttctcaactaaaaataaaataaaataaaaaataaaaaattttaaaaaaattaaaaaaaaaaaaaggctgggtgtggtggcgggtgcctgtaatcccagctactcaggaaggaggctgaggcaggagaactgcttgaacctgggaggtggaggttgcagtgagccgagattgcgccattgcattctagcctgggtgacaagagcgaaactctgtttcaacaaagaaaagaaaaagagaaaagaaaatgtggaaggcttacctaggtgtccaggcccccagccctcctcaattcctgcagatcctcagagctcaaacaactgattcctcctccccatgtccactgaggtccccttctcccacaaggccctcttccctcagactcttcctatctccaggccctgcttcactgcccacctgctttcccagtccctgtgaagggtttgccttcacatgcctcttccttcccccaggccactgtgggctcgagtaggggatgaccacctgctgcttcttcagggcgagcagctccgccggacgactcgctctgttgtccatcccaagtaccaccagggctcaggccccatcctgccaaggcgaacggatgagcacgatctcatgttgctaaagctggccaggcccgtagtgccggggccccgcgtccgggccctgcagcttccctaccgctgtgctcagcccggagaccagtgccaggttgctggctggggcaccacggccgcccggagaggcaagagctggggctctgaggccagaacctcaggaggagggggctgagggcctgaacccctgggtctgaggaaggatgggctggggactggattcctggatctgagggaggacgggctggggtcctagatgcctgggtctgtgagtctgaggggaggaggggctgggggcctggactcctgggtctaagtggggaggggctggggccaggattcttgagtctgaaggaggaggggctggggcttaggatagaaacggtcttgtatctggactcctggctccccaaggattgggggctggacccagggattactggcatattctcccttcagtgaagtacaacaagggcctgacctgctccagcatcactatcctgagccctaaagagtgtgaggtcttctaccctggcgtggtcaccaacaacatgatatgtgctggactggaccggggccaggacccttgccaggtagggtctgaacagggagagtctctgactcctgggagggaggacagggaggttatgggaaaagagcagaccctgtgcccgatcccaaactccattcccaaacccatccttgaccccaactcttacccagacctaaccccctcctcatccctatcctcaatcccatttccatcctaaccccaccccattcccatctccaagcccattttcatcccctcaccttccatgaactacaatcccaacccaagtctcactgtgccttcattctcatcccccagcccaacctcccataacctgaagtccacctccattcctaccttccagctcatacctaattccaaccccatcccatcctcgtctttatcccaacccaaccccttccttccccaccactgccccagatcccaaagtgacagctctcacgttggcacatttatttgatctctcctttctgccacccccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccccgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatgtgattgtcatgtaaggcttaacacagtgggtggtgagttctgactaaaggttacctgttgtcgtgatctgaccacgtcccggtgaaagcgtgtgtccagggaagaagtgcacagggtagcccccaatcccaaccttccatccccaacccttagggatgatggaaga

SEQ ID NO: 19 - protein, 282 aa, Homo sapiens
  ref <308>: DATABASE ACCESSION NUMBER: GenBank / BAA33404
  ref <309>: DATABASE ENTRY DATE: 2003-01-31
  ref <313>: RELEVANT RESIDUES: (1)..(282)
MQRLRWLRDWKSSGRGLTAAKEPGARSSPLQAMRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 20 - protein, 250 aa, Homo sapiens
  ref <308>: DATABASE ACCESSION NUMBER: GenBank / AAD47815
  ref <309>: DATABASE ENTRY DATE: 2000-06-26
  ref <313>: RELEVANT RESIDUES: (1)..(250)
MRILQLILLALATGLVGGETRIIKGFECKPHSQPWQAALFEKTRLLCGATLIAPRWLLTAAHCLKPRYIVHLGQHNLQKEEGCEQTRTATESFPHPGFNNSLPNKDHRNDIMLVKMASPVSITWAVRPLTLSSRCVTAGTSCLISGWGSTSSPQLRLPHTLRCANITIIEHQKCENAYPGNITDTMVCASVQEGGKDSCQGDSGGPLVCNQSLQGIISWGQDPCAITRKPGVYTKVCKYVDWIQETMKNN

SEQ ID NO: 21 - DNA, 1186 bp, Homo sapiens
  ref <308>: DATABASE ACCESSION NUMBER: GenBank / AB012917
  ref <309>: DATABASE ENTRY DATE: 2003-01-31
  ref <313>: RELEVANT RESIDUES: (1)..(1186)
aggaatctgcgctcgggttccgcagatgcagaggttgaggtggctgcgggactggaagtcatcgggcagaggtctcacagcagccaaggaacctggggcccgctcctcccccctccaggccatgaggattctgcagttaatcctgcttgctctggcaacagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagttacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctgcgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtg

SEQ ID NO: 22 - DNA, 9120 bp, Homo sapiens
  gene (2313)..(7622)
  mRNA (2313)..(2398)
  mRNA (4189)..(4263)
  mRNA (5061)..(5217)
  mRNA (5545)..(5810)
  mRNA (6627)..(6763)
  mRNA (7158)..(7622)
  ref <308>: DATABASE ACCESSION NUMBER: GenBank / AF164623
  ref <309>: DATABASE ENTRY DATE: 2000-06-26
  ref <313>: RELEVANT RESIDUES: (1)..(9120)
tgataatagtgttctctctcctcattggtcagggccccagccattgtccttgagagaatgctcgactctttatgttgtcttgacagcctcccctgagattggtcattaatgactgtgctctctctcctcattggtcagggccccagccattgtccttgagagaacctctgtcctttatggagttccacccttcttccctgggattggcccctagagacagtggttcttctcttttggttagccattgccattgtcctccgggaaagtgattatactcttttgtctaatgaccagacttggagccctccccaaggcccaggactgggttgaagggttggggaggaaaacagaaataagatgtctcccttgttcagacagtacttctcttcccttccagggtgattctgggggccccctggtgtgtgggggagtccttcaaggtctggtgtcctgggggtctgtggggccctgtggacaagatggcatccctggagtctacacctatatttgcaagtatgtggactggatccggatgatcatgaggaacaactgacctgtttcctccacctccacccccaccccttaacttgggtacccctctggccctcagagcaccaatatctcctccatcacttcccctagctccactcttgttggcctgggaacttcttggaactttaactcctgccagcccttctaagacccacgagcggggtgagagaagtgtgcaatagtctggaataaatataaatgaaggaggggccatgtctgtccatttgaagtcctcatgctggttgagactggaagaaggactcagcagtttccctatctcataggagtagaaacagagctcaaataaggccaggcacagtggctcacacctgtaatcccatcactttgggaagctgaggcaggtggatcacctgaggtcaggaactcgggaccagcctggtcaacatagtgaaaccccaactctactaaaaatgcaaaaattagccaggcatggtggcgcatgcctgtaatcccagctactcaggaggctgagacaggagaatagcatgaacccgtgaggcagaggctgcagcgagccgagattgaaccattacactccagcctgggcgacagagcgagactccatctcaaaaacaaacaaacaaaaaacccagtgctcaaataggatgagggtcttccctgagtagttactcagaaatggagtagaaaaagttacttttaataatataggccgggtgcagtggcccacgcctgtaatcccagcactttgggaggccgaggtgggaggatggcttgagctcagatttcgagatcagcctggcaacacagtgaaatcttgtcactacaaaaacacaaaaaattagctgggtgtggtggtgcgtgcctgtagtcccagctacttgggaagctgaggtgggaggatcacccgagccggggaggtggaggctgcaaagagccgagatcatgccactgcactccagcctgggcaataaagtgagaccttgtctcaaaaacaaaaacccagcaatataaataagacacatgtttcttcatctggcataatagaaatagtgcccagagcttataagcttttcaagagtccacaaaagacccgaaaaagaaaaagaaaattgttagctccaaaataccagatgaaagctgcaaagtcaacatttatgaccatttaatccaatgtccataaaacgtagcattctttccactagccaactgcagtttactttcttgtaatgaagcatacattgtatctttaatgtgggacgtggctttgttctaataagacgaagggtggagtgcaggcttggaaagcaggagagctcagcctacgtctttaatcctcctgcccaccccttggattctgtctccactgggactcaagaggtgaggagagaccatctccccaaatgcactgaagggaaactggaggagggagggagtgaggggtgatcataccagcggaggcacatttgctgagcccccccgcagtctgctctttccaagtggaccctcctggaagcctgatcccaacctcccctgcaagcaggtctgtcacccccatctctcagatgaagaaactgagccttgcaggggtggagtcccttgtccccacgtcataagggtagtcatagtagtaggaagaggaagcacctaggtttgaggccagggctggctgctgtcagaacctaggccctcccctgccttgctccacacctggtcaggggagagaggggaggaaagccaagggaagggacctaactgaaaacaaacaagctgggagaagcaggaatctgcgctcgggttccgcagatgcagaggttgaggtggctgcgggactggaagtcatcgggcagaggtctcacagcagccagtaagtgaacagctggactcgggctgcctgggcggcagggagaagcgggcaggggaagggtcagcagaggagcgaggccccagaggagccctggggtggagcacagccaagggctctgttccctttcctggactcggcttccacaggccctgacctgcctcccccaccctccggtcctgcccctgtgcctggcagcagccccacctgtgtgacatcccagcacaccccccctctccttgcaaaggagaagggagcggcctaggggaggccaggggcccacctgggctggggctgtggagagggagtggctgggacgggaggaaaaagagagacggagattagatggaagaagagggatttcaagacaaattgccagagatgcagtcagagagactgactgagagacacaaagatagaaggaattagagaaagggccacacagagccagacagagagagaagagtggagatggagacagggacgaggacagagaaaggcagacagacacatagggacagaaagagaaaaatcacacaaagtcagaattactgaatgacagggaatgacacatagaacgagacacagattcagagactcagggcagggaaaggaaggctgcagacagacagacagacagagggaggctgagacacagggagaagaggggcttggagaggtggcacaggcaggcagccagtgcctcagaggcctccggggagggccctcacacacaccccgccccggggcattaaggcagggcttggaggccagtcatcctgggcccgcccagggccgcccccctgccagcccgcctgcctggtgcctggcacctggcgctccaacccagcctacctgctgtagctgccgccactgccgtctccgccgccactgggcccccagagccccagccccagagcctgtgagtccaggaggaaagggaagctgcccctccccgtccaggtgtcagccctccccaaggacacctgtcccactcgggcacccatttctccctctgctctgtccttctctgcttgggtgggggttcctggcctctctctacacctctcacctccgatggctgtccgcagcctcagttacctctaatctccatggcttcagctgctgagctggccctctgctcccacccccgctggccagggcagcggagggcactggccctcccctcgaccagccccgcccagctttgcttggctgtccttcaaaagggcaggggtttggcggacagggcttcagcaagccgggtgatgggggtcccagacattgtctggggctgagccccctactcccctccagcagacctcaaaggctccatatcgctctgctgcgaagacaatgaaaaaggggtggctacggaacggtgtctggttccccttgtccttccaccccaagctgctggggcctggccagctctcaaggcaagaaggaaaacatcctctgacatgtgccggggaggtcccatggctgacttgaacagggccgaaccatggcttgacagctcaaagcccctcccaacgacttccacatggttcttggtatctcggaagcttctagctgtgaccaggccctctccaaggccaccctagacacctaagatatattttaagtgtttggagatctgagtgctgtgagaaacaggggatttccccaaccttgtttctcccaagtggggagcgggagcaggtgagggagagaggagagggcatgagccagcccccccctcccgatttccccgtaaagtgatgcggccccatgtccctccttgttcccagaggaacctggggcccgctcctcccccctccaggccatgaggattctgcagttaatcctgcttgctctggcaacaggtacgcaggggatgggggcagggcaggatcctccctcttgaatctctgggatcccctaaccctctgtgtctggacagtgacagggctgattccaaattacagaacaacccataaggcacctgaactggagcagtggtcatgagggcctggatgcccttctagataatccctttaaatgccaaaggaggagaggtcaagggggtcgtaaagggtcccgtggaggggctgaggaagctggagttgggggagcagtcactcaaagcgcccaggacaggggctactgaccaaccagtatggaagtatttccttttttttttttcccagagacaaagtcttgctctattgtccaggctggagtgccgtggtgccaacacggctcactgcagtcttgacttcccgggcttaagtgatccttaagccatctcagcttccccggtagctgggaccacaggcacctgccaccaagccaggctaattgtttaattgtttgtagagatgggggaggaggtctcactatgtttgcctaggctgatctagaactcctgggctcaagtaatcctcccaccttagcctctcaaagtgctgggattacaggcatgagccactgcatttgaccttatggaagtattttcatcctttaatacccgaccccagcatccagggcaacccagagggacaccagaccagggcccagaccacccactctctttctctcctccccacccccatttctgggagtcctcctggtctaccacctctccttcctgagccccttcttttgctctcaccccctccagggcttgtagggggagagaccaggatcatcaaggggttcgagtgcaagcctcactcccagccctggcaggcagccctgttcgagaagacgcggctactctgtggggcgacgctcatcgcccccagatggctcctgacagcagcccactgcctcaagccgtgggtgcgggggctggggcggtgccggggtggggggctgggaatggggagatggatggagagaagctcagggataggggtgctggtaaggggattagagatggggatgggtagtgtcagcaaggttgatgggctcgagttggtattgaaggtggggggatgaatggggttgggatggggctatggctgggaagggggcttcggtgggagacgtggaagaggttggaagcagagcgatgtttcttcatcctcaaaggtgtcactcacctctcccacccatgtctcccccgacctttcctcctccaactactgtctctcccacctcagccgctacatagttcacctggggcagcacaacctccagaaggaggagggctgtgagcagacccggacagccactgagtccttcccccaccccggcttcaacaacagcctccccaacaaagaccaccgcaatgacatcatgctggtgaagatggcatcgccagtctccatcacctgggctgtgcgacccctcaccctctcctcacgctgtgtcactgctggcaccagctgcctcatttccggctggggcagcacgtccagcccccagtgtaggagcaccagaggggaacctggcagggggtggtgaggagggagtggtcaggattgtggaagggttcagggcatcagagatgcggttcacagtgacgatgtgggataagttgagaggatgtgtggaaaacgtcaggataggggggtggggacaaaagttggggccttggagtcagacggacgggatatgcaatcatacatccataacctcctggttgtaagaccttaggcaagcagcttcacctctctgaatcttgattttcttctctataaaatgagaatgattatacccacctgtcaggattggattagagataatgtatatcaagcaactgacataaatcatttattggatagcaggctgggcaccgtggctcacgcctgtaatcccagcactttgggaggccgaggtgggaagatcacctgaggtcaggactttgataccagcctggccaacgtggtgaaatcccatctctactaaaaatgtgaaaattagttgggcgtggttgtgtgcgcctgtaatcccagctactcgggaggttgaggcaggagaatcgcttaaacttgggagacggaggttgcagtgagccaagatcacgccactgcactccagcctgggcaacagagcaagactctgtctcgaaaaaaaaaaaaaaaaagctggatagcattgctgttgctattgttacaagaagagaggtgagttggctgcgtctaaggacagggattcccccaggggcgggatcacagcaagcactgcattaggggaggtggcagggggctcattcccacagcccctcacgctgtttccacagtacgcctgcctcacaccttgcgatgcgccaacatcaccatcattgagcaccagaagtgtgagaacgcctaccccggcaacatcacagacaccatggtgtgtgccagcgtgcaggaagggggcaaggactcctgccaggtcagtgtggtctccaaccacagccccatccccatccccagcttcaatgacatctttaccgacatccacaatttcatccccaacctcaacccgccgacccctgcaactcccaatccatctcttcccctgttcccgtttctgacctcagcacaaacttcagctccatccccgtttccacaccatttccagctccaaccatccccaaactcgtttttgagcctaaccccatcctttatcccacccataatcccagctttatcgctaaacctatcacctttcccagtgcctacccatcctgtctcggccccactcctaagcaccgtccccacctcctccctggctaacaccatgctcaacgctttctctgaccgacattctctctccccgtgcccagggtgactccgggggccctctggtctgtaaccagtctcttcaaggcattatctcctggggccaggatccgtgtgcgatcacccgaaagcctggtgtctacacgaaagtctgcaaatatgtggactggatccaggagacgatgaagaacaattagactggacccacccaccacagcccatcaccctccatttccacttggtgtttggttcctgttcactctgttaataagaaaccctaagccaagaccctctacgaacattctttgggcctcctggactacaggagatgctgtcacttaataatcaacctggggttcgaaatcagtgagacctggattcaaattctgccttgaaatattgtgactctgggaatgacaacacctggtttgttctctgttgtatccccagccccaaagacagctcctggccatatatcaaggtttcaataaatatttgctaaatgagtgaatctactgagtgcttactatgtgctagaccctgatccaatggcttttattttattttattttttgacagagtctcgctctgtcacccaggctggagtacagtggtgctatctctgctcactgcaacctccacctcctgggttcaagcaattctcctgcctcagcctcctgaatagctgggattacaggtgcctaccaccacatccggctaatttttgtattttttagtagagatggggcttcaccatgttggccaggctggtctcgaactcctgacctcagatgatctgccctccttggcctcccaaactcctgggattacagacgtgagccaccgcgcccgcccggctttcatttattaattaaaagaaattaaattaattaatctatttaggagacagtcttgctctgttgcccaggctggagtgcagtaacaatcacagctcacggcaatctcaatttcctggggtcaagtgattgtcctccctcagcctccagagtagctgggactacaggcacatgccacgaagcccagctaatttttgtatttttcgtagagacagaggtctcagtatgttgccccggctagtctcaaactcctgggctcaagcagtctgtcctcctcagcctccaaaagtggtgagattacaggcatgagtcgctgtgcctggcctccaagcactttcaaatgtatcaacttaatcctcacaaaaccctgtgaggtcggtactgttttcatacctattttatagttgaagaaacagacacagagaagcaaagtcacttgctcacagtcacgtggctaggagagcaaggatctgaagcaaggcgatctcttaattaccaagtgatgttcctggagtaaggctctgtttgtttcctttcctgtaaaatgctgcatgcaaaagtataacacagtaagtaaagaagtcagttagcctgcacatactaagacctaaccaaaggagcttattgtttttctccaacttccatgataggtaattagatagtggagacctctgctggccaatatggtagccactaaccgcagctggctcttccaattaaaattacataaagccagaaatgtaactcctctgtctcacttgttatatctccaaggctggatagccacatgtgactggtggtggctggattagctagtgcatataaaacatcactgcagaaagttcagctgagcagcactgagttagatggcctctgaagaggatgtcccacggagagaatccagaactcaggatctttttttttttttttctttgcgacagagtcttgctctgtcacccaggctggagtgcagtggcgtgatctcggctcactgcaacttctgcctcccaggttcaagcaattctcctgcctcagcctccctagtagctgggactacaggcctgtgccaacatccccagctaatttttgtgtctttttagtagagatggggtttcactatgttggccaggctggtctcgaactcctgacc